(19) United States
(12) Patent Application Publication
(10) Pub. No.: US 2025/0011326 A1
(43) Pub. Date: Jan. 9, 2025

(54) FUSED HETEROCYCLIC DERIVATIVES AND THEIR USE IN THE TREATMENT OF HBV INFECTION

(71) Applicant: Janssen Sciences Ireland Unlimited Company, Co Cork (IE)

(72) Inventors: Lianzhu Liu, Shanghai (CN); Gang Deng, Shanghai (CN); Chunliang Lu, Shanghai (CN); Bingqing Tang, Shanghai (CN); Zhiguo Liu, Shanghai (CN); Zhanling Cheng, Shanghai (CN); Sandrine Céline Grosse, Turnhout (BE); Koen Vandyck, Paal-Beringen (BE); Edgar Jacoby, Vosselaar (BE); Tim Hugo Maria Jonckers, Heist-op-den-Berg (BE); Pierre Jean-Marie Bernard Raboisson, Rosieres (BE); Scott D. Kuduk, Harleysville, PA (US); Lindsey Graham Deratt, North Wales, PA (US)

(21) Appl. No.: 18/326,737

(22) Filed: May 31, 2023

(30) Foreign Application Priority Data
Dec. 1, 2021 (WO) PCT/CN2021/134798

(51) Int. Cl. C07D 471/14; A61K 45/06; A61P 31/20

(52) U.S. Cl. CPC C07D 471/14; A61K 45/06; A61P 31/20

(57) ABSTRACT

The application describes fused heterocycle derivative compounds, pharmaceutical compositions comprising these compounds, chemical processes for preparing these compounds and their use in the treatment of diseases associated with HBV infection.

### FIELD

The application relates to fused heterocyclic derivative compounds, pharmaceutical compositions comprising these compounds, chemical processes for preparing these compounds and their use in the treatment of diseases associated with HBV infection.

### BACKGROUND

Chronic hepatitis B virus (HBV) infection is a significant global health problem affecting over 5% of the world population (over 350 million people worldwide and 1.25 million individuals in the U.S.).

Despite the availability of a prophylactic HBV vaccine, the burden of chronic HBV infection continues to be a significant unmet worldwide medical problem, due to suboptimal treatment options and sustained rates of new infections in most parts of the developing world Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in pan to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress. Capsid structures also respond to environmental cues to allow un-coating after viral entry. Consistently, the appropriate timing of capsid assembly and dis-assembly, the appropriate capsid stability and the function of core protein have been found to be critical for viral infectivity.

The crucial function of HBV capsid proteins imposes stringent evolutionary constraints on the viral capsid protein sequence, leading to the observed low sequence variability and high conservation. Consistently, mutations in HBV capsid that disrupt its assembly are lethal, and mutations that perturb capsid stability severely attenuate viral replication. The high functional constraints on the multi-functional HBV core/capsid protein is consistent with a high sequence conservation, as many mutations are deleterious to function. Indeed, the core/capsid protein sequences are >90% identical across HBV genotypes and show only a small number of polymorphic residues. Resistance selection to HBV core/capsid protein binding compounds may therefore be difficult to select without large impacts on virus replication fitness.

Reports describing compounds that bind viral capsids and inhibit replication of HIV, rhinovirus and HBV provide strong pharmacological proof of concept for viral capsid proteins as antiviral drug targets.

There is a need in the art for therapeutic agents that can increase the suppression of virus production and that can treat, ameliorate, and/or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

In view of the clinical importance of HBV, the identification of compounds that can increase the suppression of virus production and that can treat, ameliorate, and/or prevent HBV infection represents an attractive avenue into the development of new therapeutic agents. Such compounds are provided herein.

### SUMMARY

The present disclosure is directed to the general and preferred embodiments defined, respectively, by the independent and dependent claims appended hereto, which are incorporated by reference herein. The present invention is directed to compounds capable of capsid assembly modulation. The compounds of the present invention may provide a beneficial balance of properties with respect to prior art compounds, e.g. they may display a different profile, display improved solubility, etc. Thus, in particular, the present disclosure is directed to a compound of Formula (I).

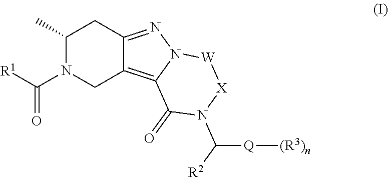

or a stereoisomeric or a tautomeric form thereof, wherein
R¹ is selected from the group consisting of phenyl, pyridyl, pyrimidinyl, pyridazinyl and pyrazinyl, each of which is substituted with 1, 2 or 3 substituents, each of said substituents independently selected from the group consisting of halo, C₁₋₄alkyl, C₃₋₆cycloalkyl CN, CF₃, CHF₂, OCHF₂, and OCF₃;
R² is hydrogen or a substituent selected from the group consisting of CHF₂, CF₃, C₁₋₄ alkyl, C₁₋₄alkylOC₁₋₄-alkyl and C₃₋₆cycloalkyl;
Q represents a ring selected from the group consisting of phenyl, a five-membered aromatic heterocyclic ring, and a six-membered aromatic heterocyclic ring,
n represents 1, 2 or 3,
each R³ independently represents a substituent selected from the group consisting of CF₃, CHF₂, CHR₂F, C₁₋₆alkyl, OC₁₋₆alkyl, OCF₃, OCF₂H, and C₃₋₆cycloalkyl;
in the event that n represents 2 or 3, two R¹ on adjacent ring atoms, together with said ring atoms, optionally form a 5-membered ring or 6-membered ring, said ring optionally comprising 1, 2, or 3 heteroatoms each independently selected from N, O and S, said ring optionally carrying one or more fluor or oxo substituents;
W is CHR⁴ and X is CHR⁵, wherein R⁴ and R⁵ each independently are selected from the group consisting of hydrogen, CONR⁶R⁷, phenyl, 5-membered heterocyclyl, 6-membered heterocyclyl, 5-membered heteroaryl, 6-membered heteroaryl, C₃₋₆cycloalkyl, C₁₋₄alkyl, C₁₋₄alkyl substituted with OH, OC₁₋₄alkyl, halo, COOH, CONR⁸R⁹ NHCOC₁₋₄alkyl, NHCOC₃₋₆cycloalkyl, 5-membered heteroaryl, 6-membered heteroaryl, SO₂NHC₁₋₄alkyl, SO₂NHC₃₋₆cycloalkyl, NHSO₂C₁₋₄alkyl or NHSO₂C₃₋₆cycloalkyl; wherein R⁴ and R⁵ are not both hydrogen;
R⁶ and R⁷ each independently are selected from the group consisting of hydrogen; C₃₋₆cycloalkyl; C₁₋₄alkyl; and C₁₋₄alkyl substituted with halo, OH or C₁₋₄alkyl; or R⁶ and R⁷ are taken together along with a nitrogen to which R⁶ and R⁷ are attached to form an optionally substituted 4 to 8 membered monocyclic heterocycyl;
R⁸ and R⁹ each independently are selected from the group consisting of hydrogen, C₃₋₆cycloalkyl; C₁₋₄alkyl; and C₁₋₄alkyl substituted with halo, OH, or OC₁₋₄alkyl, or R⁸ and R⁹ are taken together along with a nitrogen to which R⁸ and R⁹ are attached to form an optionally substituted 4 to 8 membered monocyclic heterocyclyl;
or W and X together are CR¹⁰═N or CR¹¹═CR¹²;
R¹⁰, R¹¹ and R¹² each independently are selected from the group consisting of hydrogen, halo, CONR¹³R¹⁴, phenyl, 5-membered heterocyclyl, 6-membered heterocyclyl, 5-membered heteroaryl, 6-membered heteroaryl, C₃₋₆cycloalkyl, C₁₋₄alkyl, and C₁₋₄alkyl optionally substituted with OH, OC₁₋₄alkyl, halo, COOH, CONR¹⁵R¹⁶, NHCOC₁₋₄alkyl, NHC(OC₃₋₆cycloalkyl, SO₂NHC₁₋₄alkyl, SO₂NHC₃₋₆cycloalkyl, NHSO₂C₁₋₄alkyl or NHSO₂C₃₋₆cycloalkyl;
R¹³ and R¹⁴ each independently are selected from hydrogen, C₁₋₄alkyl, C₃₋₆cycloalkyl, or R¹² and R¹⁴ are taken together along with a nitrogen to which R¹³ and R¹⁴ are attached to form an optionally substituted 4- to 8-membered monocyclic heterocyclyl,
R¹⁵ and R¹⁶ each independently are selected from the group consisting of hydrogen, C₁₋₄alkyl, and C₃₋₆cycloalkyl, or R¹⁵ and R¹⁶ are taken together along with a nitrogen to which R¹⁵ and R¹⁶ are attached to form an optionally substituted 4 to 8 membered monocyclic heterocyclyl, or a pharmaceutically acceptable salt or a solvate thereof.

Further embodiments include pharmaceutically acceptable salts and solvates of compounds of Formula (I), and stereoisomeric and tautomeric forms of the compounds of Formula (I), as well as pharmaceutically acceptable salts thereof.

In embodiments, the compounds of Formula (I) are compounds selected from those species described or exemplified in the detailed description below.

The present disclosure is also directed to pharmaceutical compositions comprising one or more compounds of Formula (I), and pharmaceutically acceptable salts and solvates of compounds of Formula (I). Pharmaceutical compositions may further comprise one or more pharmaceutically acceptable excipients or one or more other agents or therapeutics.

The present disclosure is also directed to methods of using or uses of compounds of Formula (I). In embodiments, compounds of Formula (I) are used to treat or ameliorate hepatitis B viral (HBV) infection, increase the suppression of HBV production, interfere with HBV capsid assembly or other HBV viral replication steps or products thereof. The methods comprise administering to a subject in need of such method an effective amount of at least one compound of Formula (I), and pharmaceutically acceptable salts and solvates of compounds of Formula (I). Additional embodiments of methods of treatment are set forth in the detailed description.

### DETAILED DESCRIPTION

Additional embodiments, features, and advantages of the subject matter of the present disclosure will be apparent from the following detailed description of such disclosure and through its practice. For the sake of brevity, the publications, including patents, cited in this specification are herein incorporated by reference.

In one embodiment, provided herein are compounds of Formula (I),

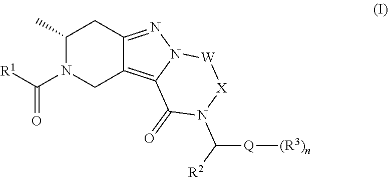

or a stereoisomeric or a tautomeric form thereof, wherein
R¹ is selected from the group consisting of phenyl, pyridyl, pyrimidinyl, pyridazinyl and pyrazinyl, each of which is substituted with 1, 2 or 3 substituents, each of said substituents being independently selected from the group consisting of halo, C₁₋₄alkyl, C₃₋₆cycloalkyl, CN, CF₃, CHF₂, OCHF₂ and OCF₃,
R² is hydrogen or a substituent selected from the group consisting of CHF₂, CF₃, C₁₋₄alkyl, C₁₋₄alkylOC₁₋₄alkyl and C₃₋₆cycloalkyl;
Q represents a ring selected from the group consisting of phenyl, a five-membered aromatic heterocyclic ring, and a six-membered aromatic heterocyclic ring;
n represents 1, 2 or 3;
each R³ independently represents a substituent selected from the group consisting of CF₃, CHF₂, CH₂F, C₁₋₆alkyl, OC₁₋₆alkyl, OCF₃, OCHF₂, and C₃₋₆cycloalkyl;
in the event that n represents 2 or 3, two R³ on adjacent ring atoms, together with said ring atoms, optionally form a 5-membered ring or 6-membered ring, said ring optionally comprising 1, 2, or 3 heteroatoms each independently selected from N, O and S, said ring optionally carrying one or more fluoro or oxo substituents,
W is CHR⁴ and X is CHR⁵, wherein R⁴ and R⁵ each independently are selected from the group consisting of hydrogen; CONR⁶R⁷; phenyl; 5-membered heterocyclyl; 6-membered heterocyclyl; 5-membered heteroaryl; 6-membered heteroaryl, C₃₋₆cycloalkyl C₁₋₄alkyl; and C₁₋₄alkyl substituted with OH, OC₁₋₄alkyl, halo, COOH, CONR⁸R⁹, NHCOC₁₋₄alkyl, NHCOC₃₋₆cycloalkyl, 5-membered heteroaryl, 6-membered heteroaryl, SO₂NHC₁₋₄alkyl, SO₂NHC₃₋₆cycloalkyl, NHSO₂C₁₋₄alkyl or NHSO₂C₁₋₄cycloalkyl; wherein R⁴ and R⁵ are not both hydrogen.
R⁶ and R⁷ each independently are selected from the group consisting of hydrogen; C₃₋₆cycloalkyl, C₁₋₄alkyl; and C₁₋₄alkyl substituted with halo, OH or OC₁₋₄alkyl, or R⁶ and R⁷ are taken together along with a nitrogen to which R⁶ and R⁷ are attached to form an optionally substituted 4 to 8 membered monocyclic heterocyclyl;
R⁸ and R⁹ each independently are selected from the group consisting of hydrogen, C₅₋₆cycloalkyl; C₁₋₄alkyl; and C₁₋₄alkyl substituted with halo, OH, or (C₁₋₄alkyl, or R⁸ and R⁹ are taken together along with a nitrogen to which R⁸ and R⁹ are attached to form an optionally substituted 4 to 8 membered monocyclic heterocyclyl;
or W and X together are CR¹⁰═N or CR¹³═CR¹²; wherein
R¹⁰, R¹¹ and R¹² each independently are selected from the group consisting of hydrogen, halo, CONR¹³R¹⁴, phenyl, 5-membered heterocyclyl, 6-membered heterocyclyl, 5-membered heteroaryl, 6-membered heteroaryl, C₃₋₆cycloalkyl, C₁₋₄alkyl, and C₁₋₄alkyl substituted with OH, OC₁₋₄alkyl, halo, COOH, CONR¹⁵R¹⁶, NHCOC₁₋₄alkyl, NHCOC₃₋₆cycloalkyl, SO₂NHC₁₋₄alkyl, SO₂NHC₃₋₆cycloalkyl, NHSO₂C₁₋₄alkyl or NHSO₂C₃₋₆cycloalkyl;
R¹³ and R¹⁴ each independently are selected from hydrogen, C₁₋₄alkyl, C₃₋₆cycloakyl, or R¹³ and R¹⁴ are taken together along with a nitrogen to which R¹³ and R¹⁴ are attached to form an optionally substituted 4- to 8-meandered monocyclic heterocyclyl.
R¹⁵ and R¹⁶ each independently are selected from hydrogen, C₁₋₄alkyl, C₃₋₆cycloalkyl, or R¹⁵ and R¹⁶ are taken together along with a nitrogen to which R¹⁵ and R¹⁶ are attached to form an optionally substituted 4 to 8 membered monocyclic heterocyclyl;
or a pharmaceutically acceptable salt or a solvate thereof.

In an additional embodiment, the present disclosure provides compounds of Formula (IA), and the stereoisomers or tautomers thereof, and the pharmaceutically acceptable salts thereof, as described herein:

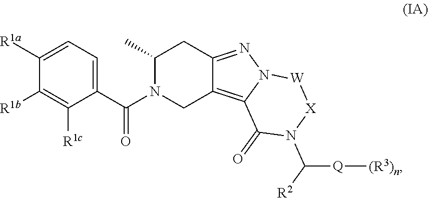

wherein R^{1a}, R^{1b}, and R^{1c}, each independently are selected from the group consisting of hydrogen, halo, C₁₋₄alkyl, C₃₋₆cycloalkyl, CN, CF₃, CHF₂, OCHF₂ and OCF₃, with at least one of R^{1a}, R^{1b}, and R^{1c} not being hydrogen, and wherein R, W, X, Q, R³ and n are as defined for Formula (I).

In a further embodiment, the present disclosure provides compounds of Formula (IB), and the stereoisomers or tautomers thereof, and the pharmaceutically acceptable salts thereof, as described herein:

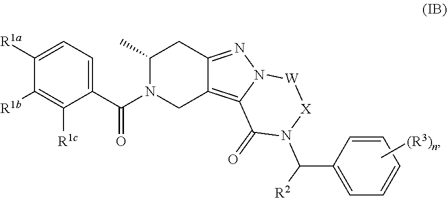

wherein R^{1a}, R^{1b}, and R^{1c}, W, X, R², R³ and n are as defined for Formula (IA).

In another embodiment, the present disclosure provides compounds of Formula (IC), and the stereoisomers or tautomers thereof, and the pharmaceutically acceptable salts thereof, as described herein:

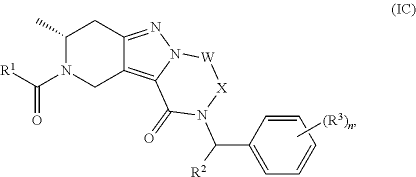

wherein R¹, W, X, R², R³ and n are as defined for Formula (I).

In a still further embodiment the present disclosure provides compounds of Formula (ID), and the stereoisomers or tautomers thereof, and the pharmaceutically acceptable salts thereof, as described herein:

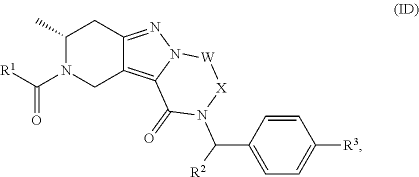

wherein R¹, W, X, R², and R³ areas defined for Formula (I).

In yet another embodiment, the present disclosure provides compounds of Formula (IE), and the stereoisomers or tautomers thereof, and the pharmaceutically acceptable salts thereof, as described herein:

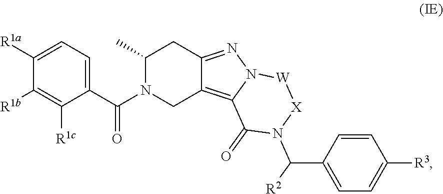

wherein R^{1a}, R^{1b}, R^{1c}, W, X, R², and R³ are as defined for Formula A).

In another embodiment, the present disclosure presents compounds of any one of formula (I), (IA), (IB), (IC), (ID), or (IE) as defined above, wherein one of R⁴ and R⁵ is H, and the other one is selected from the group consisting of CONHC₁₋₄alkyl, C₁₋₄alkyl, C₁₋₄alkyl substituted with OH, and C₁₋₄alkyl substituted with NHCOC₁₋₄alkyl.

In another embodiment, the present disclosure presents compounds of any one of formula (I), (IA), (IB), (IC), (ID), or (IE) as defined above, wherein W and X together are CH═N or CH═CH.

In another embodiment, the present disclosure presents compounds of any one of formula (IA), (IB), or (IE) as defined above, wherein R^{2a} is halo, such as chloro, R^{2b} is selected from the group consisting of halo, such as chloro, and cyano, and wherein R^{1c} is hydrogen.

In another embodiment, the present disclosure presents compounds of any one of formula (I), (IA), (IB), (IC), (ID), or (IE), wherein one R³ is OCHF₂.

In another embodiment, the present disclosure presents compounds of any one of formula (I), (IA), (IB), or (IC), wherein n is 1.

In another embodiment, the present disclosure presents compounds of any one of formula (I), (IA), (IB), or (IC), wherein n is 1 and wherein R³ is OCHF₂.

In an embodiment, the present disclosure provides compounds of any one of the formulae (I), (IA), (IB), (IC), (ID), or (IE), wherein R² is H or CH₃.

A further embodiment of the present disclosure is a compound selected from the group consisting of the compounds described in Table I below, a stereoisomer or tautomer thereof, or a pharmaceutically acceptable salt thereof.

TABLE 1

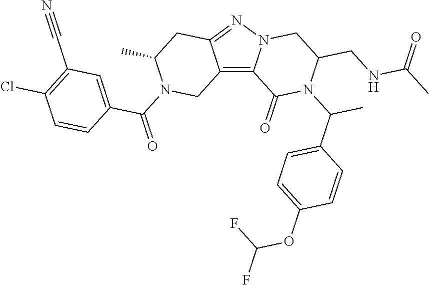 [SMILES: CC(=O)NCC1Cn2nc3c(c2C(=O)N1C(C)c1ccc(OC(F)F)cc1)CN(C(=O)c1ccc(Cl)c(C#N)c1)[C@H](C)C3]

1

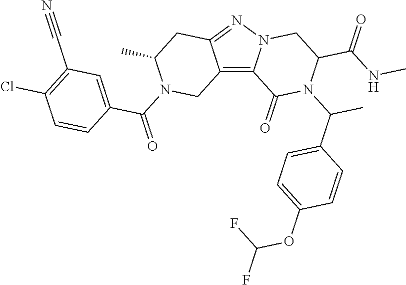 [SMILES: CNC(=O)C1Cn2nc3c(c2C(=O)N1C(C)c1ccc(OC(F)F)cc1)CN(C(=O)c1ccc(Cl)c(C#N)c1)[C@H](C)C3]

2

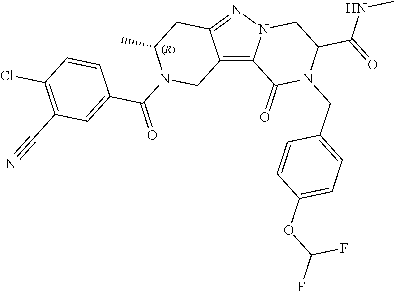

3

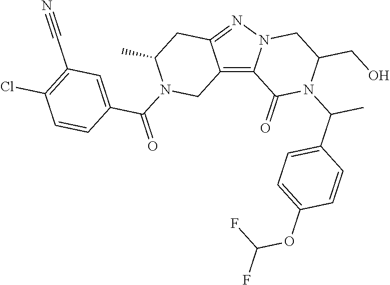 [SMILES: CC(c1ccc(OC(F)F)cc1)N1C(=O)c2c3c(nn2CC1CO)C[C@@H](C)N(C(=O)c1ccc(Cl)c(C#N)c1)C3]

4

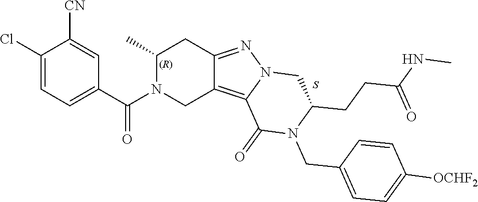

5

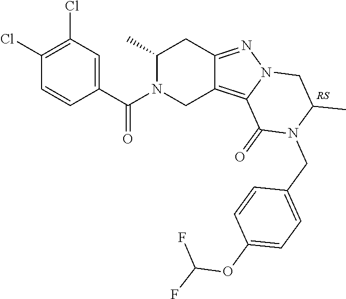

6

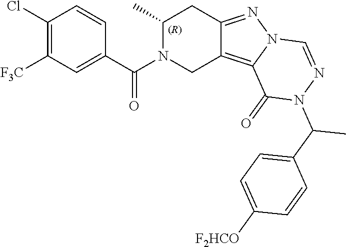

7

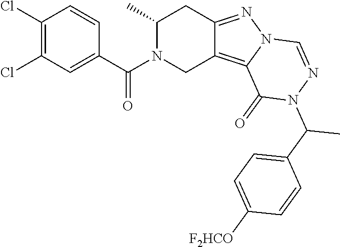 [SMILES: CC(c1ccc(OC(F)F)cc1)n1ncn2nc3c(c2c1=O)CN(C(=O)c1ccc(Cl)c(Cl)c1)[C@H](C)C3]

8

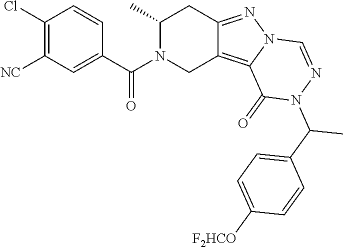 [SMILES: CC(c1ccc(OC(F)F)cc1)n1ncn2nc3c(c2c1=O)CN(C(=O)c1ccc(Cl)c(C#N)c1)[C@H](C)C3]

9

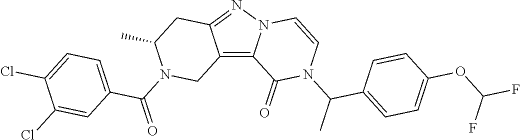 [SMILES: CC(c1ccc(OC(F)F)cc1)n1ccn2nc3c(c2c1=O)CN(C(=O)c1ccc(Cl)c(Cl)c1)[C@H](C)C3]

10

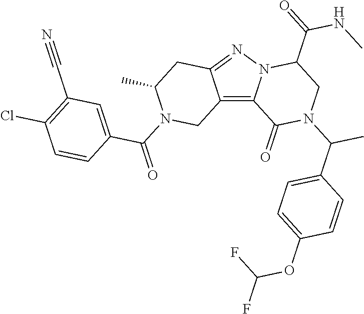 [SMILES: CNC(=O)C1CN(C(C)c2ccc(OC(F)F)cc2)C(=O)c2c3c(nn21)C[C@@H](C)N(C(=O)c1ccc(Cl)c(C#N)c1)C3]

11

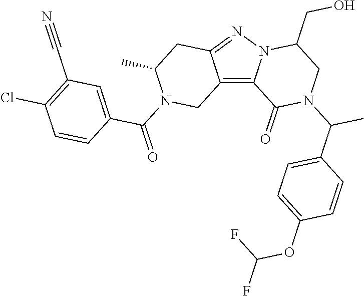 [SMILES: CC(c1ccc(OC(F)F)cc1)N1CC(CO)n2nc3c(c2C1=O)CN(C(=O)c1ccc(Cl)c(C#N)c1)[C@H](C)C3]

12

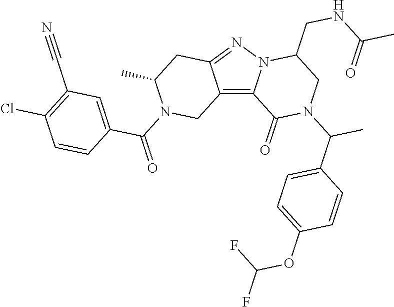 [SMILES: CC(=O)NCC1CN(C(C)c2ccc(OC(F)F)cc2)C(=O)c2c3c(nn21)C[C@@H](C)N(C(=O)c1ccc(Cl)c(C#N)c1)C3]

13

### Pharmaceutical Compositions

Also disclosed herein are pharmaceutical compositions comprising
(A) at least one compound of any one of Formula (I), (IA), (IB), (IC), (ID), or (IE), in any one of the embodiments defined above, or a pharmaceutically acceptable salt thereof, and
(B) at least one pharmaceutically acceptable excipient.

In embodiments, the pharmaceutical composition comprises at least, one additional active or therapeutic agent. Additional active therapeutic agents may include, for example, an anti-HBV agent such as an HBV polymerase inhibitor, interferon, viral entry inhibitor, viral maturation inhibitor, capsid assembly modulator, reverse transcriptase inhibitor, immunomodulatory agent such as a TLR-agonist, or any other agents that affect the HBV life cycle and/or the consequences of HBV infection. The active agents of the present disclosure are used, alone or in combination with one or more additional active agents, to formulate pharmaceutical compositions of the present disclosure.

As used herein, the term “composition” or “pharmaceutical composition” refers to a mixture of at least one compound useful within the present disclosure with a pharmaceutically acceptable carrier. The pharmaceutical composition facilitates administration of the compound to a patient or subject. Multiple techniques of administering a compound exist in the an including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the present disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the present disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth, malt, gelatin, talc, excipients such as cocoa butter and suppository waxes; oils, such as peanut oil, cottonseed oil, safflower oil, sesame oil, olive oil, corn oil and soybean oil; glycols, such as propylene glycol, polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid, pyrogen-free water; isotonic saline, Ringer's solution, ethyl alcohol; phosphate buffer solutions; and other non-toxic compatible substances employed in pharmaceutical formulations

As used herein, “pharmaceutically acceptable carrier” also includes any and all coatings, antibacterial and antifungal agents, and absorption delaying agents, and the like that are compatible with the activity of the compound useful within the present disclosure and are physiologically acceptable to the patient. Supplementary active compounds may also be incorporated into the compositions. Other additional ingredients that may be included in the pharmaceutical compositions used in the practice of the present disclosure are known in the an and described, for example in Remington's Pharmaceutical Sciences (Genaro, Ed., Mack Publishing Co., 1985, Easton, PA), which is incorporated herein by reference.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the present disclosure can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets may include a compound according to the present disclosure mixed with pharmaceutically acceptable excipients such as inert diluents, disintegrating agents, binding agents, lubricating agents, sweetening agents, flavoring agents, coloring agents and preservative agents. Suitable inert tillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract or may be coated with an enteric coating.

Capsules for oral administration include hard and soft gelatin capsules. To prepare hard gelatin capsules, compounds of the present disclosure may be mixed with a solid, semi-solid, or liquid diluent. Soft gelatin capsules may be prepared by mixing the compound of the present disclosure with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain pharmaceutically-acceptable excipients such as suspending agents (for example, sorbitol, methyl cellulose, sodium alginate, gelatin, hydroxyethylcellulose, carboxyethylcellulose, aluminum stearate gel and the like), non-aqueous vehicles, e.g., oil (for example, almond oil or fractionated coconut oil), propylene glycol, ethyl alcohol, or water, preservatives (for example, methyl or propyl p-hydroxybenzoate or sorbic acid); wetting agents such as lecithin; and, if desired, flavoring or coloring agents.

The active agents of this present disclosure may also be administered by non-oral routes. For example, the compositions may be formulated for rectal administration as a suppository. For parenteral use, including intravenous, intramuscular, intraperitoneal, or subcutaneous routes, the compounds of the present disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the present disclosure may utilize a patch formulation to affect transdermal delivery.

Compounds of the present disclosure may alternatively be administered in methods of this present disclosure by inhalation, via the nasal or oral routes. e.g., in a spray formulation also containing a suitable carrier,

### Methods of Use

The disclosed compounds are useful in the prevention or treatment of an HBV infection or of an HBV-induced disease in mammal in need thereof, more particularly in a human in need thereof.

In a non-limiting aspect, these compounds may (i) modulate or disrupt HBV assembly and other HBV core protein functions necessary for HBV replication or the generation of infectious particles, (ii) inhibit the production of infectious virus particles or infection, or (iii) interact with HBV capsid to effect defective viral particles with reduced infectivity or replication capacity acting as capsid assembly modulators. In particular, and without being bound to any particular mechanism of action, it is believed that the disclosed compounds are useful in HBV treatment by disrupting, accelerating, reducing, delaying and/or inhibiting normal viral capsid assembly and/or disassembly of immature or mature particles, thereby inducing aberrant capsid morphology leading to antiviral effects such as disruption of virion assembly and/or disassembly, virion maturation, virus egress and/or infection of target cells.

The disclosed compounds may act as a disruptor of capsid assembly interacting with mature or immature viral capsid to perturb the stability of the capsid, thus affecting its assembly and/or disassembly. The disclosed compounds may perturb protein folding and/or salt bridges required for stability, function and/or normal morphology of the viral capsid, thereby disrupting and/or accelerating capsid assembly and/or disassembly. The disclosed compounds may bind capsid and alter metabolism of cellular polyproteins and precursors, leading to abnormal accumulation of protein monomers and/or oligomers and/or abnormal particles, which causes cellular toxicity and death of infected cells. The disclosed compounds may cause failure of the formation of capsids of optimal stability, affecting efficient uncoating and/or disassembly of viruses (e.g., during infectivity). The disclosed compounds may disrupt and/or accelerate capsid assembly and/or disassembly when the capsid protein is immature. The disclosed compounds may disrupt and/or accelerate capsid assembly and/or disassembly when the capsid protein is mature. The disclosed compounds may disrupt and/or accelerate capsid assembly and/or disassembly during viral infectivity which may further attenuate HBV viral infectivity and/or reduce viral load. The disruption, acceleration, inhibition, delay and/or reduction of capsid assembly and/or disassembly by the disclosed compounds may eradicate the virus from the host organism. Eradication of HBV from a subject by the disclosed compounds advantageously obviates the need or chronic long-term therapy and/or reduces the duration of long-term therapy.

An additional embodiment of the present disclosure is a method of treating a subject suffering from an HBV infection, comprising administering to a subject in need of such treatment an effective amount of at least one compound of Formula (I).

In another aspect, provided herein is a method of reducing the viral load associated with an HBV infection in an individual in need thereof, comprising administering to the individual a therapeutically effective amount of a compound of Formula (I), or a pharmaceutically acceptable salt thereof.

In another aspect, provided herein is a method of reducing reoccurrence of an HBV infection in an individual in need thereof, comprising administering to the individual a therapeutically effective amount of a compound of Formula (I), or a pharmaceutically acceptable salt thereof.

Additionally, HBV acts as a helper virus to hepatitis delta virus (HDV), and it is estimated that more than 15 million people may be HBV/HDV co-infected worldwide, with an increased risk of rapid progression to cirrhosis and increased hepatic decompensation, than patients suffering from HBV alone (Hughes. S A. et al Lancet 2011, 378, 73-85). HDV, infects therefore subjects suffering from HBV infection. In a particular embodiment, the compounds of the invention may be used in the treatment and/or prophylaxis of HBV/HDV co-infection, or diseases associated with HBV/HDV co infection. Therefore, in a particular embodiment, the HBV infection is in particular HBV/HDV co-infection, and the mammal, in particular the human, may be HBV/HDV co-infected, or be at risk of HBV/HDV co infection.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (I), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (I) or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (I), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (I), or a pharmaceutically acceptable salt thereof.

In embodiments, the disclosed compounds are suitable for monotherapy. In embodiments, the disclosed compounds are effective against natural or native HBV strains. In embodiments, the disclosed compounds are effective against HBV strains resistant to currently known drugs.

In another embodiment, the compounds provided herein can be used in methods of modulating (e.g., inhibiting or disrupting) the activity, stability, function, and viral replication properties of HBV cccDNA.

In yet another embodiment, the compounds of the present disclosure can be used in methods of diminishing or preventing the formation of 141V cccDNA.

In another embodiment, the compounds provided herein can be used in methods of modulating (e.g., inhibiting or disrupting) the activity of HBV cccDNA

In yet another embodiment, the compounds of the present disclosure can be used in methods of diminishing the formation of HBV cccDNA

In another embodiment, the disclosed compounds can be used in methods of modulating, inhibiting, or disrupting the generation or release of HBV RNA particles from within the infected cell.

In a further embodiment, the total burden (or concentration) of HBV RNA particles is modulated. In a preferred embodiment, the total burden of HBV RNA is diminished.

In another embodiment, the methods provided herein reduce the viral load in the individual to a greater extent or at a faster rate compared to the administering of a compound selected from the group consisting of an HBV polymerase inhibitor, interferon, viral entry inhibitor, viral maturation inhibitor, distinct capsid assembly modulator, antiviral compounds of distinct or unknown mechanism, and any combination thereof.

In another embodiment, the methods provided herein cause a lower incidence of viral mutation and/or viral resistance than the administering of a compound selected from the group consisting of an HBV polymerase inhibitor, interferon, viral entry inhibitor, viral maturation inhibitor, distinct capsid assembly modulator, antiviral compounds of distinct or unknown mechanism, and combination thereof.

In another embodiment, the methods provided herein further comprise administering to the individual at least one HBV vaccine, a nucleoside HBV inhibitor, an interferon or any combination thereof.

In an aspect, provided herein is a method of treating an HBV infection in an individual in need thereof, comprising reducing the HBV viral load by administering to the individual a therapeutically effective amount of a compound of Formula (I), or a pharmaceutically acceptable salt thereof, alone or in combination with a reverse transcriptase inhibitor; and further administering to the individual a therapeutically effective amount of HBV vaccine.

An additional embodiment of the present disclosure is a method of treating a subject suffering from an HBV infection, comprising administering to a subject in need of such treatment an effective amount of at least one compound of Formula (I).

In another aspect, provided herein is a method of reducing the viral load associated with an HBV infection in an individual in need thereof, comprising administering to the individual a therapeutically effective amount of a compound of Formula (I), or a pharmaceutically acceptable salt thereof.

In another aspect, provided herein is a method of reducing reoccurrence of an HBV infection in an individual in need thereof, comprising administering to the individual a therapeutically effective amount (of a compound of Formula (I), or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I) or a pharmaceutically acceptable salt thereof.

In another aspect, provided herein is a method of reducing an adverse physiological impact of au HBV infection in an individual in need thereof, comprising administering to the individual a therapeutically effective amount of a compound of Formula (I), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (I) or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (I), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (I), or a pharmaceutically acceptable salt thereof.

In an embodiment, the methods provided herein further comprise monitoring the HBV viral load of the subject, wherein the method is carried out for a period of time such that the HBV vims is undetectable.

### Combinations

Provided herein are combinations of one or more of the disclosed compounds with at least one additional therapeutic agent. In embodiments, the methods provided herein can further comprise administering to the individual at least one additional therapeutic agent. In embodiments, the disclosed compounds are suitable for use in combination therapy. The compounds of the present disclosure may be useful in combination with one or more additional compounds useful for treating HBV infection. These additional compounds may comprise compounds of the present disclosure or compounds known to treat, prevent, or reduce the symptoms or effects of HBV infection.

In an exemplary Embodiment, Additional Active Ingredients are those that are known or discovered to be effective in the treatment of conditions or disorders involved in HBV infection, such as another HBV capsid assembly modulator or a compound active against another target associated with the particular condition or disorder involved in HBV infection, or the HBV infection itself. The combination may serve to increase efficacy (e.g., by including in the combination a compound potentiating the potency or effectiveness of an active agent according to the present disclosure), decrease one or more side effects, or decrease the required dose of the active agent according to the present disclosure in a further embodiment, the methods provided herein allow for administering of the at least one additional therapeutic agent at a lower dose or frequency as compared to the administering of the at least one additional therapeutic agent alone that is required to achieve similar results in prophylactically treating an HBV infection in an individual in need thereof.

Such compounds include but are not limited to HBV combination drugs, HBV vaccines, HBV DNA polymerase inhibitors, immunomodulatory agents, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (CTLA-4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and any other agent that affects the HBV life cycle and/or affect the consequences of HBV infection or combinations thereof.

In embodiments, the compounds of the present disclosure may be used in combination with an HBV polymerase inhibitor, immunomodulatory agents, interferon such as pegylated interferon, viral entry inhibitor, viral maturation inhibitor, capsid assembly modulator, reverse transcriptase inhibitor, a cyclophilin/TNF inhibitor, immunomodulatory agent such as a TLR-agonist, an HBV vaccine, and any other agent that affects the HBV life cycle and/or affect the consequences of HBV infection or combinations thereof. In particular, the compounds of the present disclosure may be used in combination with one or more agents (or a salt thereof) selected from the group consisting of
HBV reverse transciptase inhibitors, and DNA and RNA polymerase inhibitors including but not limited to: lamivudine (3TC, Zeffix, Heptovir, Epivir, and Epivir-HBV), entecavir (Baraclude, Entavir), adefovir dipivoxil (Hepsara, Preveon, bis-POM PMEA), tenofovir disoproxil fumarate (Viread, TDF or PMPA);
interferons, including but not limited to interferon alpha (IFN-α), interferon beta (IFN-β), interferon lambda (IFN-λ), and interferon gamma (IFN-γ);
viral entry inhibitors;
viral maturation inhibitors;
literature-described capsid assembly modulators, such as, but not limited to SAY 41-4109;
reverse transcriptase inhibitor;
an immunomodulatory agent such as a TL R-agonist; and
agents of distinct or unknown mechanism, such as but not limited to AT-61 (E)-N-(1-chloro-3-oxo-1-phenyl-3-(piperidin-1-yl)prop-1-en-2-yl)benzamide). AT-130 ((E)-N-(1-bromo-1-(2-methoxyphenyl)-3-oxo-3-(piperidin-1-yl)prop-1-en-2-yl)-4-nitrobenzamide), and similar analogs.

In embodiments, the additional therapeutic agent is an interferon. The term “interferon” or “IFN” refers to any member the family of highly homologous species-specific proteins that inhibit viral replication and cellular proliferation and modulate immune response. Human interferons are grouped into three classes; Type 1, which include interferon-alpha (IFN-α), interferon-beta (IFN-β), and interferon-omega (IFN-ω), Type II, which includes interferon-gamma (IFN-γ), and Type III, which includes interferon-lambda (IFN-λ). Recombinant forms of interferons that have been developed and are commercially available are encompassed by the term “interferon” as used herein. Subtypes of interferons, such as chemically modified or mutated interferons, are also encompassed by the term “interferon” as used herein. Chemically modified interferons include pegylated interferons and glycosylated interferons. Examples of interferons also include, but are not limited to, interferon-alpha-2a, interferon-alpha-2b, interferon-alpha-n1, interferon-beta-1a, interferon-beta-1b, interferon-lamda-1, interferon-lamda-2, and interferon-lamda-3 Examples of pegylated interferons include pegylated interferon-alpha-2a and pegylated interferon alpha-2b.

Accordingly, in one embodiment, the compounds of Formula I, can be administered in combination with an interferon selected from the group consisting of interferon alpha (IFN-α), interferon beta (IFN-β), interferon lambda (IFN-λ), and interferon gamma (IFN-γ). In one specific embodiment, the interferon is interferon-alpha-2a, interferon-alpha-2b, or interferon-alpha-n1. In another specific embodiment, the interferon-alpha-2a or interferon-alpha-2b is pegylated. In a preferred embodiment, the interferon-alpha-2a is pegylated interferon-alpha-2a (PEGASYS).

In another embodiment, the additional therapeutic agent is selected from immune modulator or immune stimulator therapies, which includes biological agents belonging to the interferon class.

Further, the additional therapeutic agent may be an agent that disrupts the function of other essential viral protein(s) or host proteins required for HBV replication or persistence.

In another embodiment, the additional therapeutic agent is an antiviral agent that blocks viral entry or maturation or targets the HBV polymerase such as nucleoside or nucleotide or non-nucleos(t)ide polymerase inhibitors. In a further embodiment of the combination therapy, the reverse transcriptase inhibitor and/or DNA and/or RNA polymerase inhibitor is Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

In an embodiment the additional therapeutic agent is an immunomodulatory agent that induces a natural, limited immune response leading to induction of immune responses against unrelated viruses. In other words, the immunomodulatory agent can affect maturation of antigen presenting cells, proliferation of T-cells and cytokine release (e.g., IL-12, IL-18, IFN-alpha, beta, and -gamma and TNF-alpha among others).

In a further embodiment, the additional therapeutic agent is a TLR modulator or a TLR agonist, such as a TLR-7 agonist or TLR-9 agonist. In further embodiment of the combination therapy, the TLR-7 agonist is selected from the group consisting of SM360320 (4-benzyl-8-hydroxy-2-(2-methoxy-ethoxy)adenine) and AZD 8848 (methyl [3-({[3-(6-amino-2-butoxy-8-oxo-7,8-dihydro-9-purin-9-yl)propyl][3-(4-morpholinyl)propyl]amino}methyl)phenyl]acetate).

In any of the methods provided herein, the method may further compose administering to the individual at least one HBV vaccine, a nucleoside HBV inhibitor, an interferon or any combination thereof. In an embodiment, the HBV vaccine is at least one of RECOMBIVAX HB, ENGERIX-B, ELOVAC B, GENEVAC-B, or SHANVAC B.

In another aspect, provided herein is method of treating an HBV infection in an individual in need thereof, comprising reducing the HBV viral load by administering to the individual a therapeutically effective amount of a compound of the present disclosure alone or in combination with a reverse transcriptase inhibitor, and further administering to the individual a therapeutically effective amount of HBV vaccine. The reverse transcriptase inhibitor may be one of Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

For any combination therapy described herein, synergistic effect may be calculated, for example, using suitable methods such as the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Methods

The application relates to a method for the preparation of a compound of Formula (I) as described herein.

The method comprises at least one step among steps a), b), c), d), e) and f):
a) reacting a compound of Formula (II),

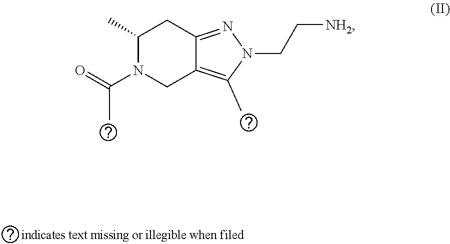 [SMILES: CC(=O)N1Cc2c(nn(CCN)c2C)C[C@H]1C]

with a compound of Formula (III),

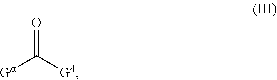

in the presence of NaBH₃CN, to form a compound of Formula (IV),

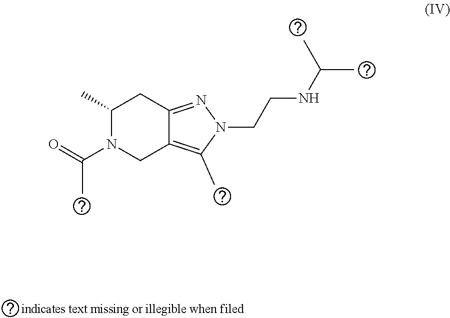

wherein
G¹ is substituted phenyl, pyridyl, pyrimidinyl, pyridazinyl and pyrazinyl;
G³ is hydrogen a group selected from CHF₂, CF₃, C₁₋₄alkyl, C₁₋₄alky OC₁₋₄alkyl and C₁₋₄cycloalkyl;
G⁴ is a substituted phenyl, or a 6- or a 5-membered aromatic heterocyclic ring;
b) reacting a compound of Formula (V),

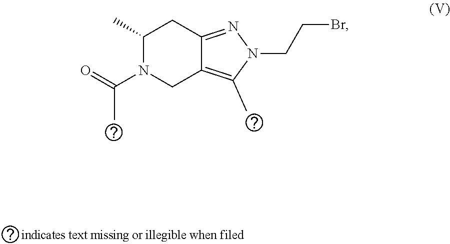 [SMILES: CC(=O)N1Cc2c(nn(CCBr)c2C)C[C@H]1C]

with a compound of Formula (VI),

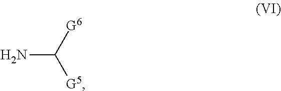

in the presence of potassium iodide (KI), to form a compound of of Formula (VII),

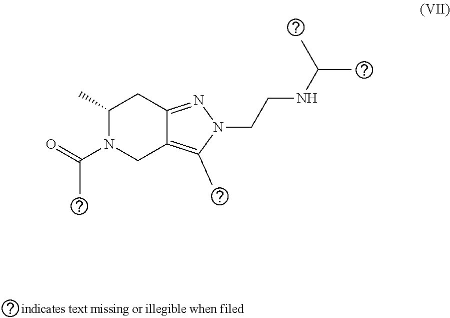

wherein G¹ is substituted phenyl, pyridyl, pyrimidinyl, pyridazinyl or pyrazinyl,
G⁵ is hydrogen or a group selected from CHF₂, CF₃, C₁₋₄alkyl, C₁₋₄alkyOC₁₋₄alkyl and C₃₋₆cycloalkyl;
G⁶ is a substituted phenyl or a 5- or a 6-membered aromatic heterocyclic ring;
c) reacting a compound of Formula (VIII),

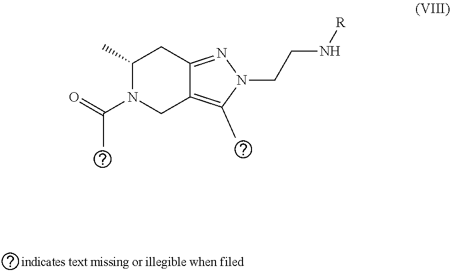

with 1,5,7-triazabicyclo[4.4.0]dec-5-en (TBD) or trimethylaluminium (Al(CH₃)₃), optionally in the presence of 1,8-diazabicyclo[5.4.0]undec-7-ene (DBU), to form a compound of Formula (IX).

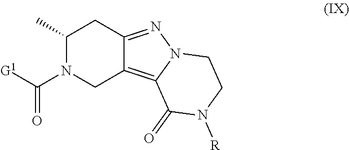

wherein
R is hydrogen or

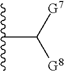

G¹ is substituted phenyl, pyridyl, pyrimidinyl, pyridazinyl, pyrazinyl or tert-butoxide;
G⁷ is a substituted phenyl or a 3- or a 6-membered aromatic heterocyclic ring;
G⁸ is hydrogen, CHF₂, CF₃, C₁₋₄alkyl, C₁₋₄alkylOC₁₋₄alkyl or C₃₋₆cycloalkyl;
d) reacting a compound of Formula (X),

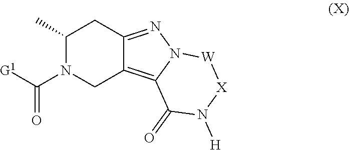

with a compound of Formula (X),

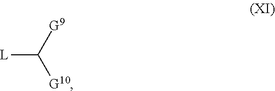

in the presence of a non-nucleophilic base, more particularly cesium carbonate (Cs₂CO₃), sodium hydride (NaH), sodium hydroxide, or sodium hydroxide/benzyltriethylammonium chloride to form a compound or Formula (XII),

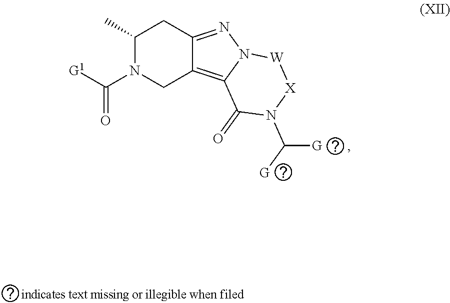

wherein G¹ is substituted phenyl, pyridyl, pyrimidinyl, pyridazinyl, or pyrazinyl;
G² is hydrogen, CHF₂, CF₃, C₁₋₄alkyl, C₁₋₄alkylOC₁₋₄alkyl or C₃₋₆cycloalkyl,
G¹⁰ is a substituted phenyl or a 5- or a 6-membered aromatic heterocyclic ring;
L is bromide, chloride or iodide;
W and X are as defined in Formula (I)—(IC);
e) reacting a compound of Formula (XIII),

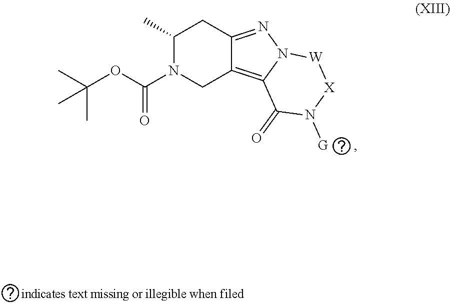

with a strong acid, more particularly hydrochloric acid (HCl) or trifluoroacetic acid (TFA), to form a compound of Formula (XIV),

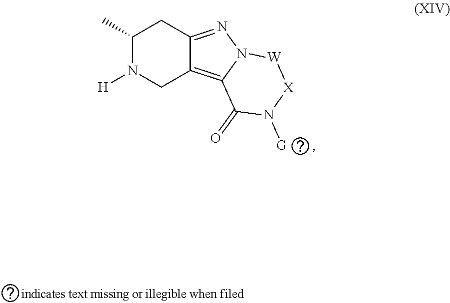

wherein G¹¹ is H, a substituted phenyl or a 5- or a 6-membered aromatic heterocyclic ring;
f) reacting a compound of Formula (XIV),

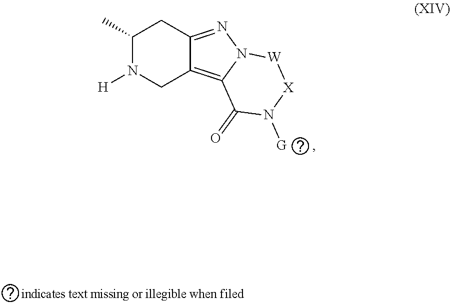

with a compound of Formula (XV),

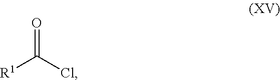

in the presence of a non-nucleophilic base, more particularly sodium carbonate (Na₂CO₃), triethylamine (Et₃N), diisopropylethylamine (DIPEA), to form a compound of Formula (XVI)

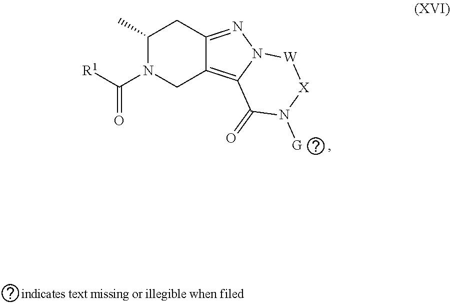

wherein R¹ is as defined for a compound of Formula (I)—(IC) and wherein G¹¹ has-been defined in step e), provided that
when the process comprises step a, the process further comprises step c,
when the process comprises step b, the process further comprises step c, and
when the process comprises step e, the process further comprises step f.

### Definitions

Listed below are definitions of various terms used to describe this present disclosure. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the applicable art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as“include,” “includes,” and “included.” is not limiting.

As used in the specification and in the claims, the term “comprising” can include the embodiments “consisting of” and “consisting essentially of” The terms “comprise(s),” “include(s)” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds. All ranges disclosed herein are inclusive of the recited endpoint and independently combinable (for example, the range of “from 50 mg to 300 mg” is inclusive of the endpoints, 50 mg and 300 ng, and all the intermediate values). The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “substantially,” cannot be limited to the precise value specified, in some cases. In at least some instances, the approximating language can correspond to the precision of an instrument for measuring the value.

The term “alkyl” as a group or as part of another group, refers to a straight- or branched-chain alkyl group having carbon and hydrogen atoms in the chain. Examples of alkyl groups include methyl (Me, which also may be structurally depicted by the symbol, “/”), ethyl (E), n-propyl, isopropyl, butyl, isobutyl, sec-butyl, tert-butyl (tBu), pentyl, isopentyl, ten-pentyl, hexyl, isohexyl, and groups that in light of the ordinary skill in the at and the teachings provided herein would be considered equivalent to any one of the foregoing examples. The term C₁₋₄alkyl as used here refers to a straight- or branched-chain alkyl group having from 1 to 4 carbon atoms in the chain. The term C₁₋₄alkyl as used here refers to a straight- or branched-chain alkyl group having from 1 to 6 carbon atoms in the chain.

The term “C₃₋₆cycloalkyl” refers to a saturated monocyclic carbocycle having from 3 to 6 ring atoms illustrative examples of cycloalkyl groups include cyclopropyl, cyclobutyl, cyclopentyl, and cyclohexyl.

The term “phenyl” represents the following moiety:

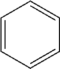

The term “heteroaryl” refers to an aromatic monocyclic or bicyclic aromatic ring system having 5 to 10 ring members and which contains carbon atoms and from 1 to 4 heteroatoms independently selected from the group consisting of N, O, and S. Included within the term heteroaryl are aromatic rings of 5 or 6 members wherein the ring consists of carbon atoms and has at least one heteroatom member. Suitable heteroatoms include nitrogen, oxygen, and sulfur. In the case of 5 membered rings, the heteroaryl ring preferably contains one member of nitrogen, oxygen or sulfur and, in addition, up to 3 additional nitrogens. In the case of 6 membered rings, the heteroaryl ring preferably contains from 1 to 4, e.g. tetrazolyl, more in particular from 1 to 3 nitrogen atoms. For the case wherein the 6 membered ring has 3 nitrogens, at most 2 nitrogen atoms are adjacent. Examples of heteroaryl groups include furyl, thienyl, pyrrolyl, oxazolyl, thiazolyl, imidazolyl, pyrazolyl, oxazolyl, thiazolyl, oxadiazolyl, triazolyl, thiadiazolyl, pyridinyl, pyridazinyl, pyrimidinyl, pyrazinyl, indolyl, isoindolyl, benzofuryl, benzothienyl, indazolyl, benzimidazolyl, benzothiazolyl, benzoxazolyl, benzisoxazolyl, benzothiadiazolyl, benzotriazolyl, quinolinyl, isoquinolinyl and quinazolinyl. Unless otherwise noted, the heteroaryl is attached to its pendant group at any heteroatom or carbon atom that results in a stable structure.

Those skilled in the art will recognize that the species of heteroaryl groups listed or illustrated above are not exhaustive, and that additional species within the scope of these defined terms may also be selected.

The term “heterocyclyl” represents a non-aromatic monocyclic or bicyclic system, unless otherwise specified, having for example, 4 to 8 ring members, more usually 5 to 6 ring members. Examples of monocyclic groups are groups containing 4 to 8 ring members, more usually, 5 or 6 ring members. Non-limiting examples of monocyclic heterocyclyl systems containing at least one heteroatom selected front nitrogen, oxygen or sulfur (N, O, S) include, but are not limited to 4-to 8-membered heterocyclyl systems such as oxetanyl, pyrrolidinyl, tetrahydrofuranyl, piperidinyl, piperazinyl, pyranyl, dihydropyranyl, tetrahydropyranyl, morpholinyl, thiomorpholinyl. Unless otherwise specified, each can be bound to the remainder of the molecule through any available ring carbon atom or nitrogen atom, and may optionally be substituted, where possible, on carbon and/or nitrogen atoms according to the embodiments. Optional substituents of 4- to 8-membered monocyclic heterocyclyl, include OH, OC₁₋₄alkyl, halo, COOH, CONHCH₃, NHCOC₁₋₄alkyl, NHCOC₃₋₆cycloalkyl, and C₁₋₄alkyl.

The term “cyano” refers to the group —CN.

The terms “halo” or “halogen” represent chloro, fluoro, bromo or iodo.

The term “oxo” represents ═O.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

The terms “para”, “meta”, and “ortho” have the meanings as understood in the art. Thus, for example, a fully substituted phenyl group has substituents at both “ortho” (o) positions adjacent to the point of attachment of the phenyl ring, both “meta” (m) positions, and the one “para” (p) position across from the point of attachment. To further clarify the position of substituents on the phenyl ring, the 2 different ortho positions will be designated as ortho and ortho” and the 2 different meta positions as meta and meta’ as illustrated below.

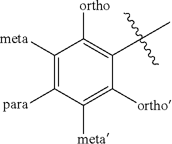

When referring to substituents on a pyridyl group, the terms “para”, “meta”, and “ortho” refer to the placement of a substituent relative to the point of attachment of the pyridyl ring. For example, the structure below is described as 3-pyridyl with the X¹ substituent in the ortho position, the X² substituent in the meta position, and X¹ substituent in the para position:

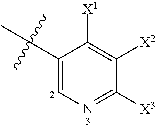

To provide a more concise description, some of the quantitative expressions given herein are not qualitied with the term “about” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

The terms “buffered” solution or “buffer” solution are used herein interchangeably according to their standard meaning. Buffered solutions are used to control the pH of a medium, and their choice, use, and function is known to those of ordinary skill in the art. See, for example, G. D. Considine, ed., Van Nostrand's Encyclopedia of Chemistry. p. 261, 5^{th} ed. (2005), describing, inter alia, buffer solutions and how the concentrations of the buffer constituents relate to the pH of the buffer. For example, a buffered solution is obtained by adding MgSO₄ and NaHCO₃ to a solution in a 10.1 w/w ratio to maintain the pH of the solution at about 7.5.

Any formula given herein is intended to represent compounds having structures depicted by the structural formula as well as certain variations or forms. In particular, compounds of any formula given herein may have asymmetric centers and therefore exist in different enantiomeric forms. All optical isomers of the compounds of the general formula, and mixtures thereof are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers

It is also to be understood that compounds that have the same molecular formula but differ in the nature or sequence of bonding of their atoms or the arrangement of their atoms in space are termed “isomers.”

Stereoisomers that are not mirror images of one another are termed “diastereomers” and those that are non-superimposable mirror images of each other are termed “enantiomers.” When a compound has an asymmetric center, for example, it is bonded to four different groups, and a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e. as (+)- or (−)-isomers respectively). A chiral compound can exist as either an individual enantiomer or as a mixture thereof. A mixture containing equal proportions of the entantiomer is called a “racemic mixture.”

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenyl nitromethane, that are likewise formed by treatment with acid or base.

Tautomeric forms may be relevant to the attainment of the optimal chemical reactivity and biological activity of a compound of interest.

The compounds of this present disclosure may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the ar.

Certain examples contain chemical structures that are depicted as an absolute enantiomer but are intended to indicate enantiopure material that is of unknown configuration. In these cases (R*) or (S*) or (*R) or (*S) is used in the name to indicate that the absolute stereochemistry of the corresponding stereocenter is unknown. Thus, a compound designated as (R*) or (*R) refers to an enantiopure compound with an absolute configuration of either (R) or (S), in cases where the absolute stereochemistry has been confirmed, the structures are named using (R), and (S).

The symbols [symbol] and [symbol] are used as meaning the same spatial arrangement in chemical structures shown herein. Analogously, the symbols [symbol] and [symbol] are used as meaning the same spatial arrangement in chemical structures shown herein.

Certain compounds of Formula (I), or pharmaceutically acceptable salts of compounds of Formula (I), may be obtained as solvates. Solvates include those formed from the interaction or complexation of compounds of the present disclosure with one or more solvents, either in solution or as a solid or crystalline form. In some embodiments, the solvent is water and the solvates are hydrates.

Reference to a compound herein stands for a reference to any one of (a) the actually recited form of such compound, and (b) any of the forms of such compound in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation: R—COOH₍ₛₒₗ₎ refers to the undissociated form of the compound in a solvent, and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH_{(aq)}, and/or R—COO⁻_{(aq)} where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the ar.

In another example, a zwitterionic compound is encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEB1.27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts” Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion “H₃NCH₂COO⁻. Zwitterions, zwitterionic compounds, inner salts and dipolar ions in the known and well established meanings of these terms are within the scope of this present disclosure, as would in any case be so appreciated by those of ordinary skill in the art. Because there is no need to name each and every embodiment that would be recognized by those of ordinary skill in the art, no structures of the zwittenionic compounds that are associated with the compounds of this present disclosure are given explicitly herein. They are, however, part of the embodiments of this present disclosure. No further examples in this regard are provided herein because the interactions and transformations in a given medium that lead to the various forms of a given compound are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorus, sulfur, fluorine, chlorine, and iodine such as ²H, ³H, ¹¹C, ¹³C, ¹⁴C, ¹⁵N, ¹⁸O, ¹⁷O, ³¹P, ³²P, ³⁵S, ¹⁸F, ¹⁶Cl, ¹²⁵I, respectively. Such isotopically labeled compounds are useful in metabolic studies (preferably with ¹⁴C), reaction kinetic studies (with, for example deuterium (i.e., D or ²H); or tritium (i.e., T or ³H)), detection or imaging techniques such as positron emission tomography (PET) or single-photon emission computed tomography (SPECT) including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this present disclosure can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety front a list of possible species for a specified variable is not intended to define the same choice of the species for the variable appearing elsewhere in other words, where a variable appears more than once, the choice of the species from a specified list is independent of the choice of the species for the same variable elsewhere in the formula, unless stated otherwise.

According to the foregoing interpretive considerations on assignments and nomenclature, it is understood that explicit reference herein to a set implies, where chemically meaningful and unless indicated otherwise, independent reference to embodiments of such set and reference to each and every one of the possible embodiments of subsets of the set referred to explicitly.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this present disclosure given according to the choices S¹ₑₓₐₘₚₗₑ is S¹ and S²ₑₓₐₘₚₗₑ is S₃, S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₁; S¹ₑₓₐₘₚₗₑ is S₁ and S²ₑₓₐₘₚₗₑ is S₄; and equivalents of each one of such choices. The shorter terminology “S¹ₑₓₐₘₚₗₑ is one of S₁ and S₂; and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein. The foregoing convention given herein for substituents extends, when applicable, to members such as R¹, R², R³, R⁴, R⁵, G¹, G², G³, G⁴, G⁵, G⁶, G⁷, G⁸, G⁹, G¹⁰, G¹¹, n, L, R, T, Q, W, X, Y, and Z and any other generic substituent symbol used herein.

Furthermore, when more than one assignment is given for any member or substituent, embodiments of this present disclosure comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this present disclosure for which Sₑₓₐₘₚₗₑ is S₁, Sₑₓₐₘₚₗₑ is S₂; Sₑₓₐₘₚₗₑ is S₁; Sₑₓₐₘₚₗₑ is one of S₁ and S₂, Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃, and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to members such as R¹, R², R³, R⁴, R⁵, G¹, G², G³, G⁴, G⁵, G⁶, G⁷, G⁸, G⁹, G¹⁰, G¹¹, N, L, R, T, Q, W, X, Y, and Z and any other generic substituent symbol used herein.

The nomenclature “Cᵢ₋ⱼ” with j>i, when applied herein to a class of substituents, is meant to refer to embodiments of this present disclosure for which each and every one of the number of carbon members, from i to j including i and j, is independently realized. By way of example, the term C₁₋₄ refers independently to embodiments that have one carbon member (C₁), embodiments that have two carbon members (C₂), embodiments that have three carbon members (C₃) and embodiments that have four carbon members (C₄).

The term Cₙ₋ₘalkyl refers to an aliphatic chain, whether straight or branched, with a total number N of carbon members in the chain that satisfies n≤N≤m, with m>n. Any disubstituent referred to herein is meant to encompass the various attachment possibilities when more than one of such possibilities are allowed. For example, reference to disubstituent -A-B—, where A≠B, refers herein to such disubstituent with A attached to a first substituted member and B attached to a second substituted member, and it also refers to such disubstituent with A attached to the second substituted member and B attached to the first substituted member.

The present disclosure includes also pharmaceutically acceptable salts of the compounds of Formula (I) preferably of those described above and of the specific compounds exemplified herein, and methods of treatment using such salts.

The term “pharmaceutically acceptable” means approved or approvable by a regulatory agency of Federal or a state government or the corresponding agency in countries other than the United States, or that is listed in the U. S. Pharmacopoeia or other generally recognized pharmacopoeia for use in animals, and more particularly, in humans.

A “pharmaceutically acceptable salt” is intended to mean a salt of a free acid or base of compounds represented by Formula (I) that are non-toxic, biologically tolerable, or otherwise biologically suitable for administration to the subject it should possess the desired pharmacological activity of the parent compound. See, generally, G. S. Paulekuhn, et al., “Trends in Active Pharmaceutical Ingredient Salt Selection based on Analysis of the Orange Book Database”, J. Med. Chem., 2007, 50; 6665-72, S. M. Berge, et al., “Pharmaceutical Salts”, J Pharm Sci., 1977, 66. 1-19, and Handbook of Pharmaceutical Salts; Properties, Selection, and Use, Stahl and Wermuth, Eds., Wiley-VCH and VCH, Zurich, 2002. Examples of pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt.

As used herein, the term “composition” or “pharmaceutical composition” refers to a mixture of at least one compound provided herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound provided herein within or to the patient such that it can perform its intended function. Typically, such constructs are carried or transported from one organ, or portion of the body, to another organ, or portion of the body. Each carrier must be “acceptable” in the sense of being compatible with the other ingredients of the formulation, including the compound provided herein, and not injurious to the patient. Some examples of materials that can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth, malt; gelatin; talc; excipients, such as cocoa butter and suppository waxes; oils, such as peanut oil, cottonseed oil, safflower oil, sesame oil, olive oil, corn oil and soybean oil glycols, such as propylene glycol; polyols, such as glycerin, sorbitol, mannitol and polyethylene glycol, esters, such as ethyl oleate and ethyl laurate, agar, buffering agents, such as magnesium hydroxide and aluminum hydroxide; surface active agents, alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound provided herein, and are physiologically acceptable to the patient. Supplementary active compounds can also be incorporated into the compositions. The “pharmaceutically acceptable carrier” can further include a pharmaceutically acceptable salt of the compound provided herein. Other additional ingredients that can be included in the pharmaceutical compositions provided herein are known in the art and described, for example in Remington's Pharmaceutical Sciences (Genaro, Ed, Mack Publishing Co., 1985, Easton, PA), which is incorporated herein by reference.

The term “stabilizer,” as used herein, refers to polymers capable of chemically inhibiting or preventing degradation of a compound disclosed herein. Stabilizers are added to formulations of compounds to improve chemical and physical stability of the compound.

The term “tablet,” as used herein, denotes an orally administrable, single-dose, solid dosage form that can be produced by compressing a drug substance or a pharmaceutically acceptable salt thereof, with suitable excipients (e.g., tillers, disintegrants, lubricants, glidants, and/or surfactants) by conventional tableting processes.

As used herein, the term “capsule” refers to a solid dosage form in which the drug is enclosed within either a hard or soft soluble container or “shell.” The container or shell can be formed from gelatin, starch and/or other suitable substances.

As used herein, the terms “effective amount,” “pharmaceutically effective amount.” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

The term “combination,” “therapeutic combination,” “pharmaceutical combination,” or “combination product” as used herein refer to a non-fixed combination or a kit of parts for the combined administration where two or more therapeutic agents can be administered independently, at the same time or separately within time intervals, especially where these time intervals allow that the combination partners show a cooperative, e.g., synergistic, effect.

The term “modulators” include both inhibitors and activators, where “inhibitors” refer to compounds that decrease, prevent, inactivate, desensitize, or down-regulate HBV assembly and other HBV core protein functions necessary for HBV replication or the generation of infectious particles.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In one embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies and/or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity and/or is lethal to the virus.

As used herein, the term “treatment” or “treating,” is defined as the application or administration of a therapeutic agent. i.e., a compound of the present disclosure (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject or individual is human.

In treatment methods according to the present disclosure, an effective amount of a pharmaceutical agent according to the present disclosure is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the compounds of the present disclosure may be ascertained by routine methods such as modeling, dose escalation studies or clinical trials, and by taking into consideration routine factors, e.g. the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day. An example of a dose of a compound is from about 1 mg to about 2,500 mg.

Once improvement of the patient's disease, disorder, or condition has occurred, the dose may be adjusted for preventative or maintenance treatment. For example, the dosage or the frequency of administration or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

HBV infections that may be treated according to the disclosed methods include HBV genotype A, B, C, and/or D infections. However, in an embodiment, the methods disclosed may treat any HBV genotype (“pan-genotypic treatment”) HBV genotyping may be performed using methods known in the art, for example, INNO-LIPA® HBV Genotyping, Innogenetics N.V., Ghent, Belgium).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Each of the relevant disclosures of all references cited herein is specifically incorporated by reference. The following examples are offered by way of illustration, and not by way of limitation.

### Examples

Exemplary compounds useful in methods of the present disclosure will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I) and Formula (Ia). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Compounds of any one of Formula (I), (IA), (IB), (IC), (ID, (ID) and (IE), may be converted to their corresponding salts using methods known to one of ordinary skill in the art. For example, an amine of Formula (I) is treated with trifluoroacetic acid, HCl, or citric acid in a solvent such as Et₂O, CH₂Cl₃, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I), (IA), (IB), (IC), (ID, (ID), and (IE), may be obtained in crystalline form by recrystallization from polar solvents (including mixtures of polar solvents and aqueous mixtures of polar solvents) or from non-polar solvents (including mixtures of non-polar solvents).

Where the compounds according to this present disclosure have at least one chiral center, they may accordingly exist as enantiomers. Where the compounds possess two or more chiral centers, they may additionally exist as diastereomers. It is to be understood that all such isomers and mixtures thereof are encompassed within the scope of the present disclosure.

Compounds represented as “stereometric mixture” (means a mixture of two or more stereoisomers and includes enantiomers, diastereomers and combinations thereof) are separated by SFC resolution.

Compounds may be obtained as single forms, such as single enantiomers, by form-specific synthesis, or by resolution. Compounds may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

### 1. General Information

### Chemical Names

Chemical names were generated using the chemistry software: ACD/ChemSketch, and may follow preferably the IUPAC rules.

### General Procedure for LCMS Methods

The High Performance Liquid Chromatography (HPLC) measurement was performed using a LC pump, a diode-array (DAD) or a UV detector and a column as specified in the respective methods.

Flow from the column was brought to the Mass Spectrometer (MS) which was configured with an atmospheric pressure ion source. It is within the knowledge of the skilled person to set the tune parameters (e.g., scanning range, dwell time . . . ) in order to obtain ions allowing the identification of the compound's nominal monoisotopic molecular weight (MW) Data acquisition was performed with appropriate software.

Compounds are described by their experimental retention times (Rt) and ions. If not specified differently, the reported molecular ion corresponds to the [M+H]⁺ (protonated molecule) and/or [M−H]⁻ (deprotonated molecule) bi case the compound was not directly ionizable the type of adduct is specified (i.e. [M+NH₄]⁺, [M+HCOO]⁻ etc.). All results were obtained with experimental uncertainties that are commonly associated with the method used. Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica., “Q-Tof” Quadrupole Time-of-flight mass spectrometers. “CLND”; ChemiLuminescent Nitrogen Detector, “ELSD” Evaporative Light Scanning Detector.

### NMR Analysis

¹H NMR spectra were recorded on 1) a Bruker DPX 400 MHz spectrometer or 2) a Bruker Avance 400 MHz spectrometer or c) Bruker Avance III 400 MHz spectrometer or d) Bruker Avance 600 MHz spectrometer or e) a Bruker Avance NEO 400 MHz spectrometer or f) Bruker model AVIII 400 spectrometer g) ZKNJ BIXI-1 300 MHz, Bruker Avance III 400 MHz or h) Bruker AVANCE Neo 400 MHz.

NMR spectra were recorded at ambient temperature unless otherwise stated. Data are reported as follow: chemical shift in parts per million (ppm) relative to TMS (δ=0 ppm) on the scale, integration, multiplicity (s=singulet, d=doublet, t=triplet, q=quartet, quin=quintet, sext=sextet, sept=septet, m multiplet, b=broad, or a combination of these), coupling constant(s) J in Hertz (Hz).

### MS Analysis

Mass spectra were obtained on a Shimazu LCMS-2020 MSD or Agilent 1200/G6110A MSD using electrospray ionization (ESI) in positive mode unless otherwise indicated.

### 2. Abbreviations

TABLE 7

| | |
|---|---|
| μw | Microwave |
| ADDP | 1,1′-(Azodicarbonyl)dipiperidine |
| AlMe₃ | Trimethylaluminium |
| aq. | Aqueous |
| atm | Atmosphere |
| Boc₂O | Di-tert-butyl dicarbonate |
| BOC | tert-Butyloxycarbonyl |
| BODIPY | Boron-dipyrromethene |
| Br | broad |
| CA | Capsid assembly |
| DAST | (Diethylamino)sulfur trifluoride |
| DBU | 1,8-Diazabicyclo[5.4.0]undec-7-ene |
| DCE | 1,2-Dichloroethane |
| DCM | Dichlomethane |
| dd | Doublet of doublets |
| DEA | Diethylamine |
| DIPE | Diisopropyl ether |
| DIPEA/DIEA | N,N-diisopropylethylamine |
| DMF | Dimethylformamide |
| DMF-DMA | Dimethylformamide-dimethylacetal |
| DMSO | Dimethylsulfoxide |
| DNA | Deoxyribonucleic Acid |
| EDCl | N-(3-Dimethylaminopropyl)-N′-ethylcarbodiimide |
| | hydrochloride |
| ESI | Electrospray ionization |
| Et₃N | Triethylamine |
| Et₂O | Ether, diethyl ether |
| EtOAc/EA | Ethyl acetate |
| EtOH | Ethanol |
| FCC | Normal-phase silica gel chromatography |
| g | grams |
| h/hr | hour |
| HBV | Hepatitis B virus |
| HOAc | Acetic acid |
| HPLC | High Performance Liquid Chromatography |
| Hz | Hertz |
| i-PrNH₂ | Isopropylamine |
| i-PrOH/IPA | Isopropyl alcohol |
| KO/Bu | Potassium tert-butoxide |
| LAH | Lithium aluminum hydride |
| LCMS | Liquid Chromatography Mass Spectrometry |
| LHMDS, LiHMDS | Lithium bis(trimethylsilyl)amide |
| M | molar |
| m | multiplet |
| MeCN/ACN | Acetonitrile |
| MeOH | Methanol |
| mg | milligrams |
| MHz | megahertz |
| min | Minutes |
| mL | Milliliters |
| μL | microliters |
| mmol | Millimole |
| μmol | micromole |
| MS | Mass spectra |
| MsCl | Mesityl chloride |
| m/z | Mass to charge ratio |
| N | normal |
| NAOAc/AcONa | Sodium acetate |
| NMR | Nuclear Magnetic Resonance |
| o/n | Overnight |
| PCR | Polymerase chain reaction |
| PE | Petroleum ether |
| PMPA | 9-(2-Phosphonyl-methoxypropyl)adenine |
| ppm | Parts per million |
| ppt | precipitate |
| Py | pyridine |
| RNA | Ribonucleic Acid |
| Rₜ | Retention time |
| rt | Room temperature |
| s | singlet |
| sat | Saturated |
| SFC | Supercritical Fluid Chromatography |
| t | triplet |
| T₃P | Propanephosphonic acid anhydride |
| TBAI | Tetrabutylammonium iodide |
| TBD | 1,5,7-Triazabicyclo[4.4.0]dec-5-ene |
| TEA | triethylamine |
| TFA | Trifluoroacetic acid |
| THF | Tetrahydrofuran |
| TLC | Thin Layer Chromatography |
| TLR | Toll-like receptor |
| TNF | Tumor necrosis factor |
| V or volumes | Volume in milliliters of solvent per gram of |
| | substrate |
| Δ | Heating under reflux |

### Experimental Procedures

### Intermediate Int A

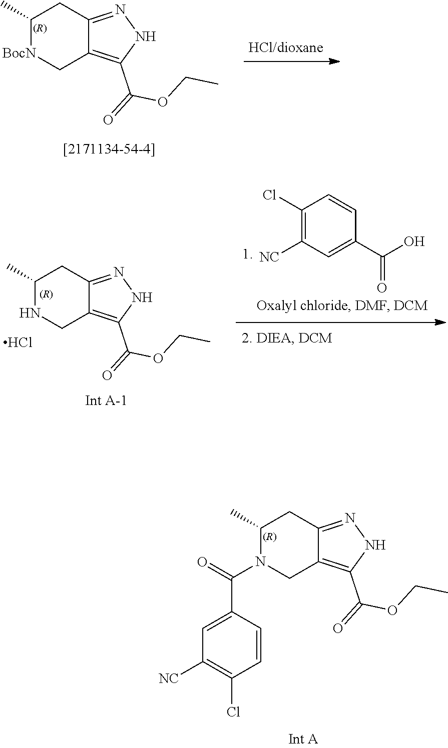

### Intermediate Int A-1:

### (R)-Ethyl 6-methyl-4,5,6,7-tetrabydro-2H-pyrazolo[4,3-c]pyridine-3-carboxylate hydrochloride

A mixture of (R)-5-tert-butyl 3-ethyl 6-methyl-6,7-dihydro-2H-pyrazolo[4,3-c]pyridine-3,5(4H)-dicarboxylate (10.0 g, 100% purity, 323 mmol) in 4 M hydrochloride in dioxane (100 mL) was stirred at room temperature for 2 hours under nitrogen atmosphere. The mixture was concentrated under reduced pressure to give the title compound (8:0 g, 95% purity from HNMR, 96% yield) as yellow solids without further purification. LC-MS (ESI): R_{T}=0.5 min, mass calcd. for C₁₀H₁₅N₃O₂ 209.1, m/z found 210.2 [M+H]⁺. ¹HNMR (400 MHz, DMSO-d₆) δ 9.68 (br s, 1H), 9.50 (br s, 1H), 4.36-4.17 (m, 5H), 3.75-3.50 (m, 1H), 3.05 (d, J=16.0 Hz, 1H), 2.75-2.69 (m, 1H), 1.45-1.37 (m, 3H), 1.34-1.26 (m, 3H).

### Intermediate Int A:

### (R)-Ethyl 5-(4-chloro-3-cyanobenzoyl)-6-methyl-4,5,6,7-tetrahydro-2H-pyrazolo[4,3-c]pyridine-3-carboxylate

To a mixture of 4-chloro-3-cyanobenzoic acid (5.9 g, 100% purity, 32.5 mmol) in dichloromethane (60 mL) was added dropwise N,N-dimethylformamide (235 mg, 3.22 mmol) and oxalyl dichloride 4 mL, 47.3 mmol) at 0° C. After stirred at room temperature for 2 hours under nitrogen atmosphere, the mixture was concentrated under reduced pressure. The resulting residue and (R)-ethyl 6-methyl-4,5,6,7-tetrahydro-2H-pyrazolo[4,3-c]pyridine-3-carboxylate hydrochloride Int A-1 (8.0 g, 95% purity, 30.9 mmol) was dissolved in dichloromethane (60 mL), followed by addition of N-ethyl-N-isopropylpropan-2-amine (16 mL, 96.8 mmol) dropwise at 0° C., After stirred tor 1 hour, the mixture was poured into water (60 mL) and adjusted to pH 5˜6 with 1M hydrochloride aqueous solution and extracted with dichloromethane (60 mL) twice. The combined organic layers were concentrated under reduced pressure. The resulting residue was purified by C18 column (acetonitrile:water(+0.02% ammonium acetate)=10% to 75%) to give the title compound (0.3 g, 90% purity, 69.1% yield) as yellow solids. LC-MS (ESI), R_{T}=1.38 min., mass calcd, for C₁₈H₁₇ClN₄O₃ 372.1, m/z found 373.1 [M+H]⁺. ¹HNMR (400 MHz, CDCl₃) δ 10.81 (br s, 1H), 7.75 (s, 1H), 7.61 (s, 2H), 5.76-5.36 (m, 1H), 4.82-4.17 (m, 4H), 3.19-2.95 (m, 1H), 2.71 (d, J=16.0 Hz, 1H), 1.38 (s, 3H), 1.26 (s, 3H).

### Intermediate Int B

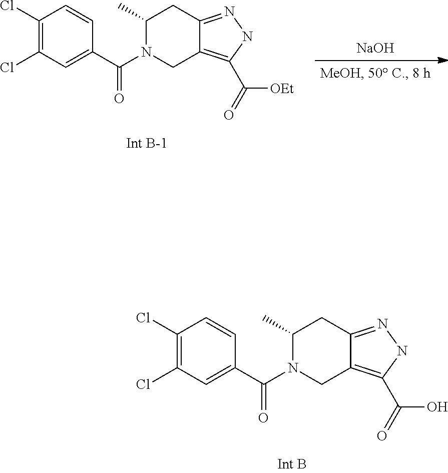 [SMILES: CCOC(=O)c1[nH]nc2c1CN(C(=O)c1ccc(Cl)c(Cl)c1)[C@H](C)C2 ; "NaOH" ; "MeOH, 50° C., 8 h" ; C[C@@H]1Cc2n[nH]c(C(=O)O)c2CN1C(=O)c1ccc(Cl)c(Cl)c1]

### Intermediate Int B-1:

### (R)-ethyl 4-(3,4-dichlor)benzoyl)-6-methyl-4,5,6,7-terrahydro-2H-pyrazolo[4,3-c]pyridine-3-carboxylate

Intermediate Int B-1 was synthesized in an analogous way to Intermediate Int A. ¹HNMR (400 MHz, DMSO-d6) δ 13.55 (br s, 1H), 7.76-7.73 (m, 2H), 7.45 (d, J=9.2 Hz, 1H), 5.52-5.14 (m, 1H), 4.59-4.01 (m, 4H), 2.98 (app s, 1H), 2.74-2.51 (m, 1H), 1.45-0.99 (m, 6H).

### Intermediate Int B:

### (R)—S-(3,4-Dichlorobenzoyl)-6-methyl-4,5,6,7-tetrahydro-2H-pyrazolo[4,3-c]pyridine-3-carboxylic acid

To a mixture of (R)-ethyl 5-(3,4-dichlorobenzoyl)-6-methyl-4,5,6,7-tetrahydro-2H-pyrazolo[4,3-c]pyridine-3-carboxylate Int B-1 (2.0 g, 100% purity, 5.23 mmol) in methanol (30 mL) was added sodium hydroxide (525 mg, 13.1 mmol) in water (6 mL) at room temperature. After stirred at 50° C. for 8 hours, the mixture was concentrated under reduced pressure. The residue was diluted with water (20 mL), adjusted to pH 4-5 with 1 M hydrochloride aqueous solution, and extracted with ethyl acetate (50 mL) twice. The combined organic layers were washed brine (40 mL), dried over Na₂SO₄₍ₛ₎, and filtered. The filtrate was concentrated to give the desired product (1.8 g, 95% purity, 92.3% yield) as white solids LC-MS (ESI): R_{T}=1.25 min, mass calcd. for C₁₅H₁₃Cl₂N₃O₃ 353.0, m/z found 354.3 [M+H]⁺. ¹H NMR (400 MHz, DMSO-d₆) δ 13.25 (br s, 2H), 7.75 (d, J=8.4 Hz, 2H), 7.46 (dd, J=8.0, 1.2 Hz, 1H), 5.39-5.22 (m, 1H), 4.45 (s, 1H), 4.15-4.08 (m, 1H), 2.99-2.90 (m, 1H), 2.55-2.45 (m, 1H), 1.10 (s, 3H).

### Compound 1B and 1D

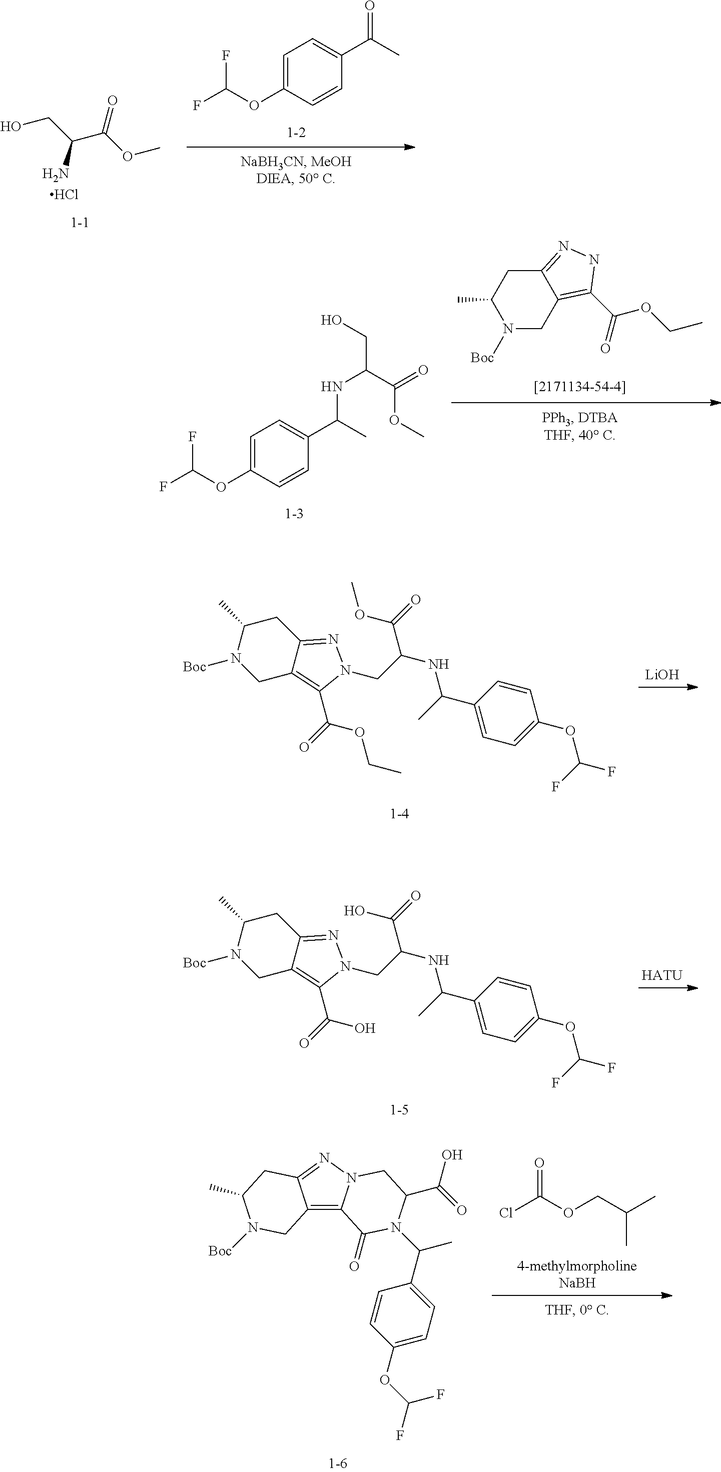

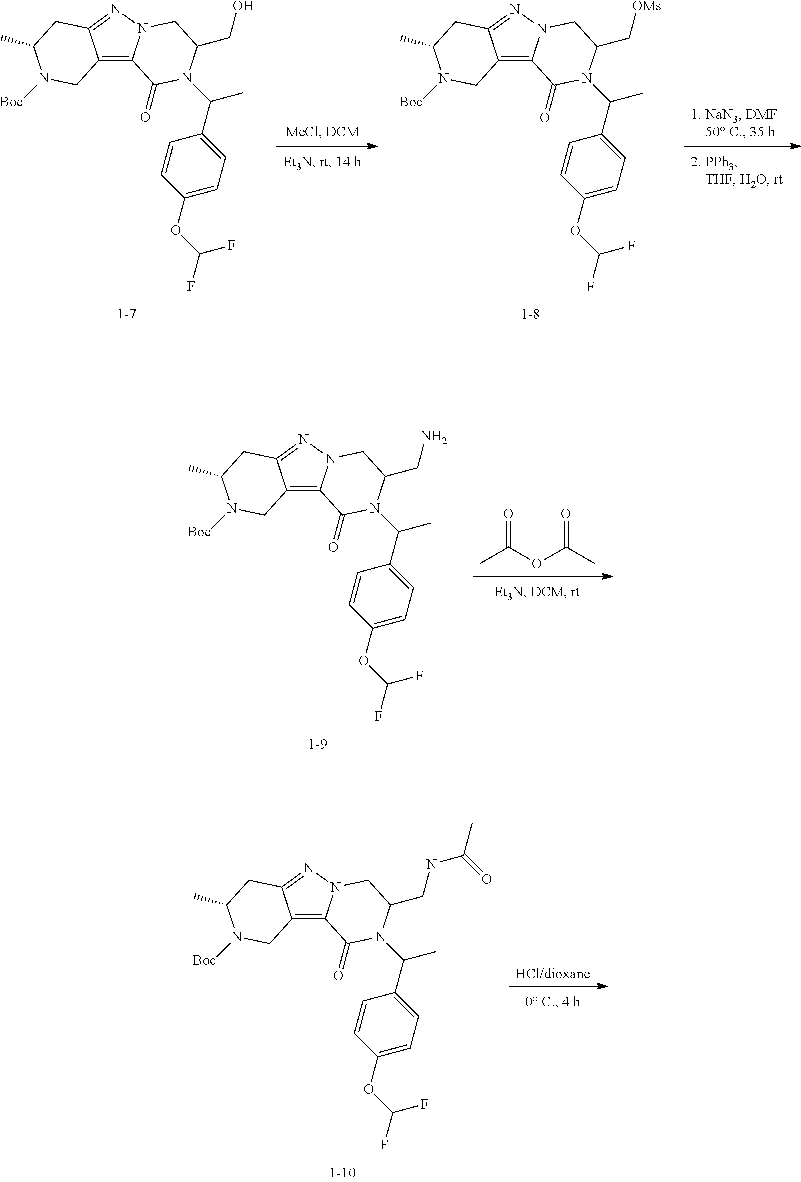

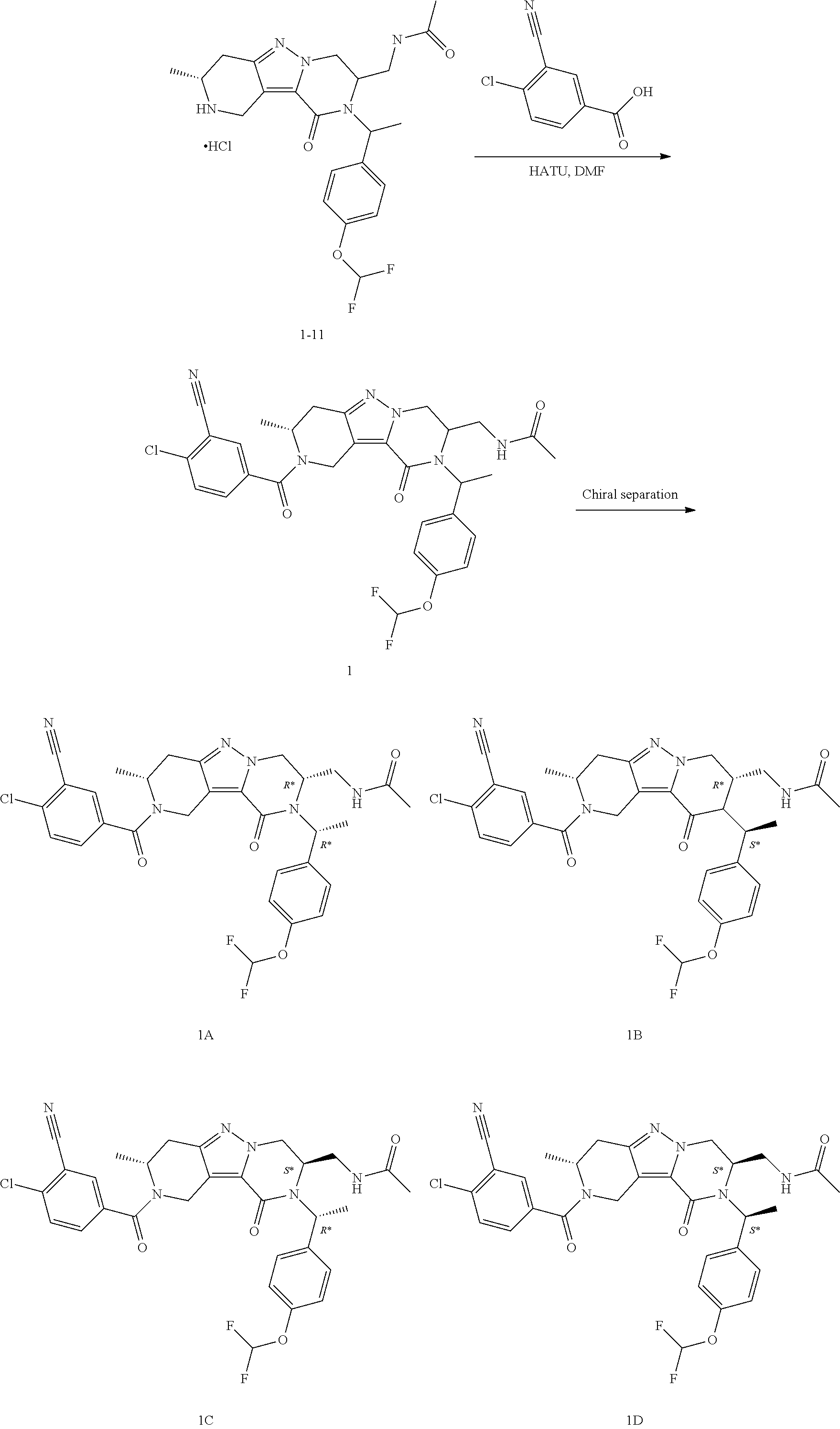

### Intermediate 1-3:

### Methyl 2-((1-(4-(difluoromethoxy)phenyl)ethyl)amino)-3-hydroxypropanoate

To a solution of (S)-methyl 2-amino-3-hydroxypropanoate hydrochloride 1-1 (6 g, 50.4 mmol) in methanol (50 mL) was added N-ethyl-N-isopropylpropan-2-amine (8.5 mL, 51.43 mmol). After stirred at room temperature for 30 minutes, 1-(4-(difluoromethoxy)phenyl)ethanone 1-2 (4.6 g 24.7 mmol) and sodium cyanoborohydride (2.4 g, 38.2 mmol) were added at room temperature. After stirred at 50° t C overnight, the mixture was quenched with 0.5 M hydrochloride aqueous solution (30 mL) and extracted with ethyl acetate (60 ml) twice. The combined organic layer was washed with brine (60 mL), dried over Na₂SO₄₍ₛ₎ and filtered. The filtrate was concentrated and purified by C18 column (acetonitrile:water=35% to 60%) to give the title compound (2.5 g, 90% purity, 31.5% yield) as white solids. LC-MS (ESI) R_{T}=1.49 min, mass calcd. for C₁₃H₁₇F₂NO₄ 289.3, m/z found 290.2 [M+H]⁺. ¹H NMR (400 MHz, CDCl₃) δ 7.32-7.27 (m, 2H), 7.09-7.06 (m, 2H), 6.49 (t, J=73.2 Hz, 1H), 3.84-3.80 (m, 1H), 3.78-3.71 (m, 2), 3.65 (s, 2H), 3.62-3.48 (m, 2H) 3.36-3.34 (m, 0.6H), 3.20-3.17 (m, 0.4H), 2.78 (br s, 0.5H), 2.47 (br s, 0.5H), 1.37 (d, J=6.4 Hz, 3H).

### Intermediate 1-4:

### (6R)-5-tert-Butyl 3-ethyl 2-(2-((1-(4-(difluoromethoxy)phenyl)ethyl)amino)-3-methoxy-3-oxopropyl)-6-methyl-6,7-dihydro-2H-pyrazolo[4,3-c]pyridine-3,5(4H)-dicarboxylate

To a mixture of methyl 2-((1-(4-(difluoromethoxy)phenyl ethyl)amino)-3-hydroxypropanoate 1-3 (2.4 g, 90% purity, 7.47 mmol) and triphenylphosphine (4.9 g, 18.7 mmol) in tetrahydrofuran (50 mL) was added (E)-di-tert-butyl diazene-1,2-dicarboxylate (4.3 g, 18.7 mmol) at 0° C., followed by addition of (R)-tert-butyl 3-ethyl 6-methyl-6,7-dihydro-2H-pyrazolo[4,3-c]pyridine-3,5(4H)-dicarboxylate (3.0 g, 92% purity, 8.92 mmol). After stirred at 40° C. overnight, the mixture was cooled to room temperature and concentrated under reduced pressure to get a residue, which was purified by C18 column (acetonitrile:water=50% to 75%) to give the title compound (1.5 g, 100% purity, 34.6% yield) as yellow oil. LC-MS (ESI): R_{T}=1.89 min, mass calcd. for C₂₈H₃₈F₂N₄O₇ 580.6, m/z found 581.0 [M+H]⁺. ¹H NMR (400 MHz, CDCl₃) δ 7.21 (d, J=8.4 Hz, 1M), 7.02 (d, J=8.4 Hz, 1H), 6.96-6.94 (m, 2H), 6.47 (t, J=74 Hz, 1H), 5.05 (br s, 1H), 4.91-4.65 (m, 3H), 4.41-4.23 (m, 2H), 4.15-4.09 (m, 2H), 3.76-3.46 (m, 5H), 3.00-2.89 (m, 1H), 2.58-2.54 (m, 1H), 1.51-1.49 (m, 9H), 1.40-1.35 (m, 3H), 1.26-1.23 (m, 3H) 1.16-1.09 (m, 3H).

### Intermediate 1-5:

### (6R)-5-(tert-Butoxycarbonyl)-2-(2-carboxy-2-((1-(4-(difluoromethoxy)phenyl)ethyl)amino)ethyl)-6-methyl-4,5,6,7-tetrahydro-2H-pyrazolo[4,3-c]pyridine-3-carboxylic acid

To the solution of (6R)-5-tert-butyl 3-ethyl 2-(2-((1-(4-(difluoromethoxy)phenyl)ethyl)amino)-3-methoxy-3-oxopropyl)-6-methyl-6,7-dihydro-2H-pyrazolo[4,3-c]pyridine-3,5(4H)-dicarboxylate 1-4 (1.5 g, 100% purity, 2.58 mmol) in methanol (4 mL) and tetrahydrofuran (12 mL) was added lithium hydroxide hydrate (589 ng, 14.0 mmol) in water (4 mL) at 0° C. After stirred at 30° C. for 1 hour, the mixture was diluted with water (15 mL), acidified with 0.5 N hydrochloride aqueous solution to pH˜5 and extracted with ethyl acetate (15 mL) twice. The combined organic layers were washed with brine (15 mL), dried over Na₂SO₄₍ₛ₎ and filtered. The filtrate was concentrated to give the title compound (1.2 g, 98% purity, 84.5% yield) as yellow oil. LC-MS (ESI): R_{T}=1.29 min, mass calcd. for C₂₅H₃₂F₂N₄O₇ 538.5, m/z found 537.0 [M−H]⁻.

### Intermediate 1-6:

### (3R)-2-(tert-Butoxycorbonyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-8-carboxylic acid

A mixture of (6R)-5-(tert-butoxycarbonyl)-2-(2-carboxy-2-((1-(4-(difluoromethoxy)phenyl)ethyl)amino)ethyl)-6-methyl-4,5,6,7-tetrahydro-2H-pyrazolo[4,3-c]pyridine-3-carboxylic acid 1-5 (1.2 g, 98% purity, 2.18 mmol), N-ethyl-N-isopropylpropan-2-amine (1.5 g, 16 mmol), 2-(3H-[1,2,3]triazolo[4,5-b]pyridin-3-yl)-1,1,3,3-tetramethylisouroniumhexafluorophosphate(V)(1.1 g, 2.89 mmol) in N,N-dimethylformamide (70 mL) was stirred at 30° C. for 14 hours. The mixture was acidified to pH=6 with hydrochloride aqueous solution and extracted with ethyl acetate (50 mL) twice. The combined organic layers were washed with water (×3), brine, dried over Na₂SO₄₍ₛ₎ and filtered. The filtrate was concentrated under reduced pressure to give a residue, which was purified by C18 column (acetonitrile:water=40% to 60%) to give the title compound (700 mg, 94% purity, 71% yield) as yellow oil. LC-MS (ESI): R_{T}=1.34 min, mass calcd. for C₂₅H₂₀F₂N₄O₆ 520.5, m/z found 519.0 [M−H]⁻.

### Intermediate 1-7:

### (3R)-tert-Butyl 9-(1-(4-(difluoromethoxy)phenyl)ethyl)-8-(hydroxymethyl)-3-methyl-10-oxo-3,4,7,8,9,10-hexahydropyrido[4′,3′:3,4]pyrazolo[1,5-a]pyrazine-2(1H)-carboxylate

To the solution of (3R)-2-(tert-butoxycarbonyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-8-carboxylic acid 1-6 (700 mg, 94% purity, 1.26 mmol) and 4-methylmorpholine (612 mg, 6.05 mmol) in tetrahydrofuran (15 mL) was added isobutyl chloroformate (50) mg, 3.66 mmol) at 0° C. After stirred at 0° C. for 1 hour, sodium borohydride (229 mg, 6.05 mmol) was added and stirring continued for 1 hour. The mixture was quenched with saturated ammonium chloride aqueous solution (15 mL), diluted with ethyl acetate (15 mL), washed with water (15 mL) twice, dried over Na₂SO₄₍ₛ₎ and concentrated under vacuum to leave a residue. The residue was purified by C18 (acetonitrile:water=40% to 65%) to give the desired product (350 mg, 97% purity, 53% yield) as yellow oil. LC-MS (ESI): R_{T}=1.63 min, mass calcd. for C₂₅H₃₂F₂N₄O₄ 506.5, m/Z found 507.1 [M+H]⁺.

### Intermediate 1-8:

### (3R)-tert-Butyl 9-(1-(4-(difluoromethoxy)phenyl)ethyl)-3-methyl-8-(((methylsulfonyl)oxy)methyl)-10-oxo-3,4,7,8,9,10-hexahydropyrido[4′,3′:3,4]pyrazolo[1,5-a]pyrazine-2(1H)-carboxylate

To a solution of (3R)-tert-butyl 9-(1-(4-(difluoromethoxy)phenyl)ethyl)-8-(hydroxymethyl)-3-methyl-10-oxo-3,4,7,8,9,10-hexahydropyrido[4′,3′:3,4]pyrazolo[1,5-a]pyrazine-2(1H)-carboxylate 1-7 (350 mg, 97% purity, 0.670 mmol) in dichloromethane (10 ml) were added triethylamine (210 mg, 2.08 mmol) and mesyl chloride (120 mg, 1.05 mmol) at 0° C. The reaction was stirred at room temperature for 14 hours. The mixture was diluted with dichloromethane (10 mL) and washed with water (25 mL). The organic layer was washed with brine (25 mL), dried over Na₂SO₄₍ₛ₎, and concentrated under vacuum to give die residue, which was purified by C18 column (acetonitrile:water=45%-70%) to give the desired product (300 mg, 97% purity, 74.3% yield) as yellow solids LC-MS (ESI): R_{T}=1.70 min, mass calcd. for C₂₆H₃₄F₂N₄O₇S 584.6, m/z found 585.0 [M+H]⁺.

### Intermediate 1-9:

### (3R)-tert-Butyl 8-(aminomethyl)-9-(1-(4-(difluoromethoxy)phenyl)ethyl)-3-methyl-10-oxo-3,4,7,8,9,10-hexahydropyrido[4′,3′:3,4]pyrazolo[1,5-a]pyrazine-2(1H)-carboxylate

To a solution of (3R)-tert-butyl 9-(1-(4-(difluoromethoxy)phenyl)ethyl)-3-methyl-8-(((methylsulfonyl)oxy)methyl)-10-oxo-3,4,7,8,9,10-hexahydropyrido[4′7:3,4]pyrazolo[1,5-a]pyrazine-2(1H)-carboxylate 1-9 (300 mg, 97% purity, 0.498 mmol) in N,N-dimethylformamide (12 ml) was added sodium azide (100 mg, 1.54 mmol). After stirred at 50° C. for 36 hours, the reaction mixture was cooled down to room temperature, diluted with water (2 mL) and tetrahydrofuran (10 mL), followed by addition of triphenylphosphine (485 mg, 1.85 mmol). After stirred for 1 hour, the mixture was diluted with ethyl acetate (15 mL), washed with water (15 mL), dried over Na₂SO₄₍ₛ₎ and concentrated under vacuum to give a residue. The crude residue was purified by silica gel column chromatography (petroleum ether ethyl acetate=10:1 to dichloromethane methanol=10:1) to give the title compound (195 mg, 78% purity, 60.4% yield) as white solids. LC-MS (ESI) R_{T}=1.55 min, mass calcd. for C₂₅H₃₃F₂N₅O₄ 505.6, m/z found 506.3 [M+H]⁺.

### Intermediate 1-10:

### (3R)-tert-Butyl 8-(acetamidomethyl)-9-(1-(4-(difluoromethoxy)phenyl)ethyl)-3-methyl-10-oxo-3,4,7,8,9,10-hexahydropyrido[4′,3′:3,4]pyrazolo[1,5-a]pyrazine-2(1H)-carboxylate

To a solution of (3R)-tert-butyl 8-(aminomethyl)-9-(1-(4-(difluoromethoxy)phenyl)ethyl)-3-methy-10-oxo-3,4,7,8,9,10-hexahydropyrido[4′,3′:3,4]pyrazolo[1,5-a]pyrazine-2(1H)-carboxylate 1-9 (1-5 mg, 78% purity, 0.301 mmol) in dichloromethane (5 mL) was added acetic anhydride (160 mg, 1.57 mmol) and triethylamine (160 mg, 1.58 mmol) at 0° C. After stirred at that temperature for 1 hour, the reaction mixture was poured into water (10 mL), extracted with ethyl acetate (10 mL) for three times. The combined organic layers were washed with brine (15 ml), dried over Na₂SO₄₍ₛ₎, and filtered. The filtrate was concentrated and purified by silica gel column chromatography (petroleum ether ethyl acetate=1:1 to dichloromethane:methanol=10:1) to give the title compound (150 mg, 100% purity from LCMS, 74.7% yield) as white solids. LC-MS (ESI): R_{T}=1.63 min, mass calcd. for C₂₇H₃₅F₂N₅O₅ 547.6, m/z found 548.0 [M+H]⁺.

### Intermediate 1-41:

### N-(((3R)-9-(1-(4-(Difluoromethoxy)phenyl)ethyl)-3-methyl-10-oxo-1,2,3,4,7,8,9,10-octahydropyrido[4′,3′:3,4]pyrazolo[1,5-a]pyrazin-1-yl)methyl)acetamide hydrochloride

To the solution of (3R)-tert-butyl 8-(acetamidomethyl)-9-(1-(4-(difluoromethoxy phenyl)ethyl)-3-methyl-10-oxo-3,4,7,8,9,10-hexahydropyrido[4′,3′:3,4]pyrazolo[1,5-a]pyrazine-2(1H)-carboxylate 1-10 (150 mg, 100% purity, 0.274 mmol) in 1,4-dioxane (0.5 mL) was added 4 M hydrochloride in dioxane (3 mL) at 0° C. After stirred at 0° C. for 4 hours, the reaction mixture was concentrated to give the title compound (150 mg, 86% purity from LCMS, 97.3% yield) as white oil LC-MS (ESI): R_{T}=1.47 min, mass calcd. for C₂₂H₂₈ClF₂N₅O₃ 483.9, m/z found 448.0 [M+H]⁺.

### Compound 1:

### N-(4(3R)-2-(4-Chloro-3-cyanobenzoyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazin-8-yl)methyl)acetamide

A mixture of N-(((3R)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazin-8-yl)methyl)acetamide hydrochloride 1-11 (150 mg, 86% purity, 0.267 mmol), N-ethyl-N-isopropylpropan-2-amine (180 mg, 1.39 mmol), 4-chloro-3-cyanobenzoic acid (84 mg, 0.463 mmol) 2-(3H-[1,2,3]triazolo[4,5-b]pyridin-3-yl)-1,1,3,3-tetramethylisouronium hexafluorophosphate(V) (204 mg, 0.537 mmol) in N,N-dimethylformamide (5 mL) was stirred at 30° C. for 14 hours. The mixture was acidified to pH=6 with hydrochloride aqueous solution and extracted with ethyl acetate (10 mL) twice. The combined organic layers were washed with water, brine (10 mL), dried over Na₂SO₄₍ₛ₎ and filtered. The filtrate was concentrated under reduced pressure to give a residue, which was purified by C18 column (acetonitrile:water=40% to 60%) to give the title compound (165 ng, 100% purity, 88.9% yield) as white solids. LC-MS (ESI): R_{T}=1.58 min, mass calcd. for C₃₀H₁₉ClF₂N₆O₄ 611.0, m/z found 611.0 [M+H]⁺.

### Compounds 1B and 1D:

### N-(((3R,8R*)-2-(4-chloro-3-cyanobenzoyl)-9-((S*)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazin-8-yl)methyl)acetamide (1B), and

### N-(((3R,8S*)-2-(4-chloro-cyanobenzoyl)-9-((S*)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazin-8-yl)methyl)acetamide (1D)

A racemate of N-(((3R)-2-(4-chloro-3-cyanobenzoyl)-9-(1-(4-(difluoromethoxy)phenyl)ethyl)-3-methyl-10-oxo-1,2,3,4,7,8,9,10-octahydropyrido[4′3′:3,4]pyrazolo[1,5-a]pyrazin-8-yl)methyl)acetamide compound 1 (187 mg, 100% purity, 0.306 mmol) was separated by chiral Prep HPLC separation condition: (Column: Chiralpak IA 5 μm 20*250 mm, Mobile Phase: CO₂:MeOH (0.2% DEA)=65:35 at 3.0 ml/min. Col. Temp 40° C.; Wavelength: 214 nm. Back pressure: 100 bar) to give compound 1C (18.5 ng, 99.6% purity, 9.9% yield, 97.7% stereopure) as white solids, compound 1D (20.4 mg, 98.3% purity, 10.7% yield, 98.3% stereopure) as white solids and a fragment including two peaks (60 mg). The fragment (60 g) was separated again by chiral Prep. HPLC separation condition: (Column: Chiralpak IB 5 μm 20*250 mm; Mobile Phase: Hex:EtOH=50:50 at 30 ml min; Temp: 30° C.; Wavelength: 254 nm) to give compound IA (6.8 mg, 99.9% purity, 3.6% yield, 98.4% stereopure) as white solids and compound 18 (9.3 mg, 99.5% purity, 4.9% yield, 100.0% stereopure) as white solids.

### Compound 1A:

LC-MS (ESI): R_{T}=3.534 min, mass calcd. For C₃₀H₂₀ClF₂N₆O₄ 611.0, m/z found 611.2 [M+H]⁺ Chiral analysis (Column: Chiralpak IA 5 μm 4.6*250 mm; Mobile Phase: Chiralpak IA 5 um 4.6*250 mm; Mobile Phase: CO₂:MeOH (0.2% DEA) 65.35 at 3 g/min; Temp: 40° C.; Wavelength: 214 nm; Back Pressure: 100 Bar; R_{T}=2.63 min). ¹H NMR (400 M Hz, CDCl₃) 7.74 (s, 1H), 7.60-7.57 (m, 4H), 7.14 (d, J=8.4 Hz, 2H), 6.52 (t, J=76.8 Hz, 1H), 5.94 (br s, 1H), 5.44-5.41 (m, 1H), 4.90-4.41 (m, 3H), 4.36-4.16 (t, 3H), 3.11 (br s, 1H), 2.79-2.55 (m, 3H), 1.87 (s, 3H), 1.67-1.65 (m, 3H), 1.27-1.26 (m, 3H). ¹⁹F NMR (376 MHz, CDCl₃) −81.00 (s, 1F), −81.05 (s, 1F).

### Compound 1B:

LC-MS (ESI): R_{T}=3.590 min, mass calcd. For C₃₀H₂₉ClF₂N₆O₄ 611.0, m/z found 611.2 [M+H]⁺ Chiral analysis (Column: Chiralpak IA 5 μm 4.6*250 mm; Mobile Phase: Chiralpak IA 5 um 4.6*250 mm; Mobile Phase CO₂:MeOH (0.2% DEA)=65:35 at 3 g/min; Temp: 40° C., Wavelength: 214 nm; Back Pressure 100 Bar; R_{T}=2.98 min). ¹H NMR (400 M Hz, CDCl₃) 7.75 (s, 1H), 7.60 (s, 2H), 7.41 (d, J=8.4 Hz, 2H), 7.09 (d, J=8.4 Hz, 2H), 6.51 (t, J=76.4 Hz, 1H), 5.99 (br s, 1H), 5.83-5.80 (m, 1H), 5.40-4.53 (m, 3H), 4.22-4.19 (m, 1H), 3.85-3.76 (m, 3H), 3.08 (br s, 1H), 2.79-2.64 (m, 2H), 1.98 (s, 3H), 1.83-1.81 (m, 3H), 1.28-126 (m, 3H). ¹⁹F NMR (376 MHz, CDCl₃) −81.12 (s, 2F).

### Compound 1C:

LC-MS (ESI): R_{T}=3.628 min, mass calcd. For C₃₀H₂₉ClF₂N₆O₄ 611.0, m/z found 611.3 [M+H]⁺. Chiral analysis (Column: Chiralpak IA 5 μm 4.6*250 mm; Mobile Phase: Chiralpak IA 5 um 4.6*250 mm. Mobile Phase: CO₂:MeOH (0.2% DEA)=65:35 at 3 g/min; Temp: 40° C.; Wavelength: 214 nm; Back Pressure: 100 Bar. R_{T}=4.12 min). 1H NMR (400 M Hz, CDCl₃) 7.75 (s, 1H), 7.61 (s, 2H), 7.43-7.41 (m, 2H), 7.10 (d, J=8.4 Hz, 2H), 6.51 (t, J=70.8 Hz, 11), 6.00 (br s, 1H), 5.82-5.79 (m, 1H), 5.70-5.42 (m, 1H), 4.6.2-4.29 (m, 2H), 4.22-4.18 (m, 1H), 3.87-3.84 (m, 1H), 3.79-3.68 (m, 2H), 3.02 (br s, 1H), 2.77-2.65 (m, 2H), 1.98 (s, 3H), 1.83-1.81 (m, 3H), 1.30-1:29 (m, 3H). ¹⁹F NMR (376 MHz. CDCl₃) −81.10 (s, 2F).

### Compound 1D:

LC-MS (ESI): R_{T}=3.534 min, mass calcd. For C₃₀H₂₉ClF₂N₆O₄ 611.0, m/z found 611.2 [M+H]⁺. Chiral analysis (Column: Chiralpak IA 5 μm 4.6*250 mm, Mobile Phase: Chiralpak IA 5 μm 4.6*250 mm; Mobile Phase; CO₂:MeOH (0.2% DEA)=65:35 at 3 g/min; Temp: 40° C., Wavelength: 214 nm; Back Pressure: 100 Bar, R_{T}=5.81 min). ¹H NMR (400 M Hz, CDCl₃) 7.74 (s, 1H), 7.61 (s, 2H), 7.56 (br s, 2H), 7.14 (d, J=8.4 Hz, 2H), 6.53 (t, 1-73.6 Hz 114), 5.93 (br s, 1H), 5.70-5.39 (m, 2H), 4.63-4.41 (m, 3H), 4.22-4.16 (m, 2H), 3.04 (br s, 1H), 2.74-258 (m, 31), 1.86 (s, 3H), 1.69-1.67 (m, 3H), 1.29-1.27 (m, 3H). ¹⁹F NMR (376 MHz, CDCl₃) −81.03 (s, 2F).

### Compound 2B

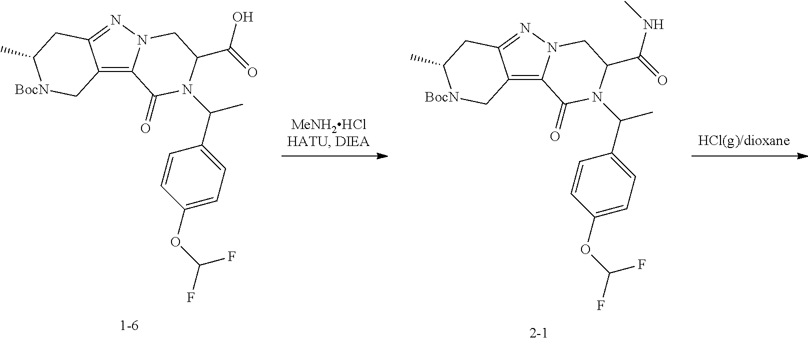 [SMILES: CC(c1ccc(OC(F)F)cc1)N1C(=O)c2c3c(nn2CC1C(=O)O)C[C@@H](C)N(C(=O)OC(C)(C)C)C3 ; "MeNH2·HClHATU, DIEA" ; CNC(=O)C1Cn2nc3c(c2C(=O)N1C(C)c1ccc(OC(F)F)cc1)CN(C(=O)OC(C)(C)C)[C@H](C)C3 ; "HCl(g)/dioxane"]

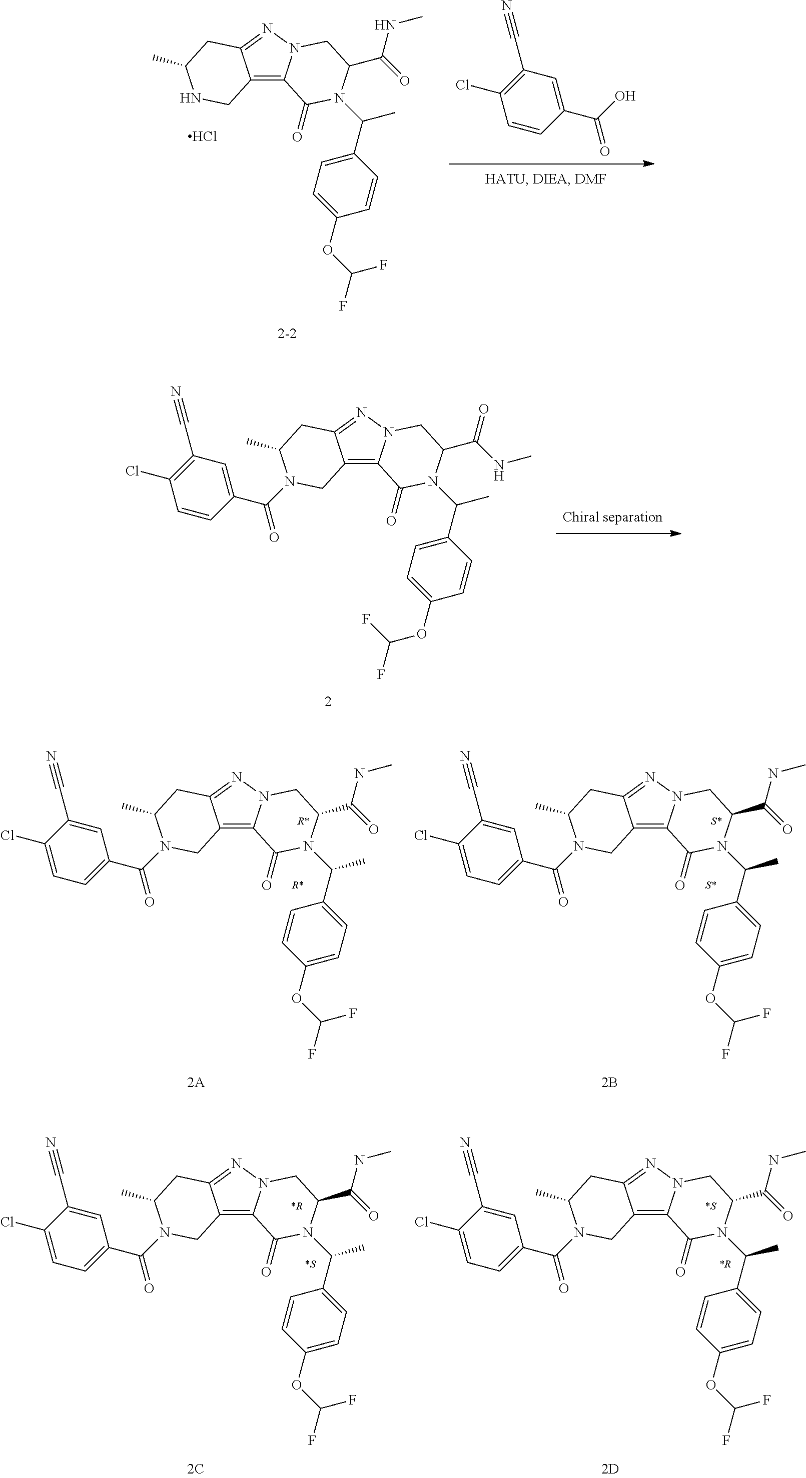

### Intermediate 2-1:

### (3R)-tert-Butyl 9-(1-(4-(difluoromethoxy)phenyl)ethyl)-3-methyl-8-(methylcarbamoyl)-10-oxo-3,4,7,8,9,10-hexahydropyrido[4′,3′:3,4]pyrazolo[1,5-a]pyrazine-2(1H)-carboxylate

A mixture of (3R)-2-(tert-butoxycarbonyl)-9-(1-(4-(difluoromethoxy)phenyl)ethyl-3-methyl-10-oxo-1,2,3,4,7,8,9,10-octahydropyrido[4′,3′:3,4]pyrazolo[1,5-a]pyrazine-8-carboxylic acid 1-6 (1.2 g, 64% purity, 1.48 mmol), N-ethyl-N-isopropylpropan-2-amine (1.71 g, 13.2 mmol), nethylamine hydrochloride (300 mg, 444 mmol) and 243N-[1,2,3]thiazolo[4,5-b]pyridin-3-yl)-1,1,3,3-tetramethylisouronium hexafluorophosphate(V) (842 mg, 2.21 mmol) in N,N-dimethylformamide (15 mL) was stirred at 0° C. under nitrogen for 2 hours. The mixture was acidified to pH=6 with 0.05 M hydrochloride aqueous solution and extracted with ethyl acetate (50 mL) twice. The combined organic layers were washed with water (60 mL) for three times and brine (60 mL), dried over Na₂SO₄₍ₛ₎ and filtered. The filtrate was concentrated under reduced pressure to get a residue, which was purified by C18 column (acetonitrile:water:=50% to 65%) to give the title compound (570 ng, 90% purity from HNMR, 65% yield) as white solids. ¹HNMR (400 MHz, CDCl₃) δ 7.48 (d, J=8.0 Hz, 1H), 7.36-7.33 (m, 1H), 7.17-7.12 (m, 2H) 6.52 (t, J=73.2 Hz, 1H), 6.23-6.11 (m, 1H), 5.27-4.64 (m, 4H), 4.31-4.16 (m, 2H), 4.04-3.92 (m, 1H), 2.98-2.86 (m, 1H), 2.86-2.77 (m, 2H), 2.59-2.54 (m, 1H), 2.26-2.22 (m, 1H), 1.60-1.59 (m, 3H), 1.50 (s, 9H) 1.14-1.05 (m, 3H).

### Intermediate 2-2:

### (3R)-9-(1-(4-(Difluoromethoxy)phenyl)ethyl)-N,3-dimethyl-10-oxo-1,2,3,4,7,8,9,10-octahydropyrido[4′,3′:3,4]pyrazolo[1,5-a]pyrazine-8-carboxamide hydrochloride

A solution of (3R)-tert-butyl 9-(1-(4-(difluoromethoxy)phenyl)ethyl)-3-methyl-8-(methylcarbamoyl-10-oxo-3,4,7,8,9,10-hexahydropyrido[4′,3′:3,4]pyrazolo[1,5-a]pyrazine-2(1H)-carboxylate 2-1 (400 mg, 90% purity, 0.675 mmol) in 4 M hydrochloride (g) in dioxane (20 mL, 80 mmol) was stirred at 20° C. for 2 hours. The mixture was concentrated to give the title product (320 mg, 98% purity from LCMS. 99% yield) as yellow solids. R_{T}=1.26 min and 1.33 min, mass calcd, for C₂₁H₂₆ClF₂N₅O₃ 433.2 m/z found 434.2[M+H]⁺

### Compound 2:

### (3R)-2-(4-Chloro-3-cyanobenzoyl)-9-(1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-8-carboxamide

A mixture of (3R)-9-(1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-8-carboxamide hydrochloride 2-2 (320 mg, 98% purity, 0.667 mmol), N-ethyl-N-isopropylpropan-2-amine (776 mg, 6.00 mmol), 4-chloro-3-cyanobenzoic acid (133 mg, 0.732 mmol) and 2-(3H-[1,2,3]triazol[4,5-b]pyridin-3-yl)-1,1,3,3-tetramethylisouronium hexafluorophosphate(V) (381 mg, 1.00 mmol) in N,N-dimethylformamide (6 mL) was stirred at 30° C. under nitrogen for 14 hours. The mixture was acidified to pH=6 with 0.05M hydrochloride aqueous solution and extracted with ethyl acetate (20 mL) twice. The combined organic layers were washed with water (36 mL) for three times and brine (30 mL), dried over Na₂SO₄₍ₛ₎ and filtered. The filtrate was concentrated under reduced pressure to give a residue, which was purified by C18 column (acetonitrile:water: 65% to 70%) to give the title compound (280 mg, 98% purity from LCMS, 69% yield) as white solids. R_{T}=1.59 min, mass calcd. for C₂₉H₂₇ClF₂N₆O₄596.2 m/z found 597.0 [M+H]⁺.

### Compound 2B:

### (3R,8S*)-2-(4-chloro-3-cyanobenzoyl)-9-((S*)-1-(4-(difluoromethoxy)phenyl)ethyl)-N,3-dimethyl-10-oxo-1,2,3,4,7,8,9,10-octahydropyrido[4′,3′:3,1]pyrazolo[1,5-a]pyrazine-8-carboxamide (28)

(3R)-2-(4-chloro-3-cyanobenzoyl)-9-(1-(4-(difluoromethoxy)phenyl)ethyl)-N,3-dimethyl-10-oxo-1,2,3,4,7,8,9,10-octahydropyrido[4′,3′:3,4]pyrazol[1,5-a]pyrazine-8-0.15 carboxamide compound 2 (280 mg, 98% purity, 0.460 mmol) was separated by chiral Prep. HPLC (separation condition: Column: Chiralpak OA 5 μm 20*250 mm; Mobile Phase: Hex:EtOH=40:60 at 12 mL/min; Temp: 30° C.; Wavelength: 254 nm) to afford compound 2A (9.6 mg, 98.3% purity, 3.4% yield, 100% stereopure), compound 2B (26.1 mg, 99.1% purity, 9.4% yield, 99.9% stereopure), compound 2C (29.4 mg, 98.6% purity, 10.5% yield, 99.4% stereopure) and compound 2D (17.9 mg, 98.0% purity, 6.4% yield, 96.7% stereopure) as white solids

### Compound 2A:

R_{T}=8.234 min, mass calcd. for C₂₉H₂₇ClF₂N₆O₄ 596.2 m/z found 597.2 [M+H]⁺. Chiral HPLC (Column: Chiralpak IA 5 μm 4.6*250 mm; Mobile Phase: Hexane:EtOH=40:60 at 1.0 mL/min; Temp: 30° C.; Wavelength: 254 nm, R_{T}=4.938 min). ¹HNMR (400 MHz, DMSO-d₆) δ 8.18-8.10 (m, 1H), 7.87-7.83 (m, 2H), 7.57 (br s, 1H), 7.41-7.01 (m, 5H), 5.77-5.68 (m, 1H), 5.53-5.20 (m, 1H), 4.53-4.33 (m, 3H), 4.30-4.08 (m, 2H), 3.00-2.81 (m, 1H), 2.66-2.58 (m, 1H), 2.18 (s, 3H), 1.57-1.50 (m, 3H), 1.20-1.09 (m, 3H). ¹⁹F NMR (376 MHz, DMSO-d₆) δ−8233 (s, 2F).

### Compound 2B:

R_{T}=3.349 min, mass calcd. for C₂₉H₂₇ClF₂N₆O₄ 596.2 m/z found 597.1 [M+H]⁺. Chiral HPLC (Column: Chiralpak IA 5 μm 4.6*250 mm; Mobile Phase; Hexane. EtOH=40:60 at 1.0 mL/min, Temp: 30° C., Wavelength: 254 nm, R_{T}=7.687 min). ¹HNMR (40 MHz, DMSO-d₆) δ 8.20-8.11 (m, 2H), 7.87-7.82 (m, 2H), 7.41 (br s, 2H), 7.24-7.05 (m, 3H), 5.84-5.69 (m, 1H), 5.53-5.19 (m, 1H), 4.56-4.10 (m, 5H), 2.95-2.83 (m, 1H), 2.68-2.61 (m, 1H), 2.58 (d, J=4.4 Hz, 3H), 1.38 (br s, 3H), 1.22-1.12 (m, 3H). ¹⁹F NMR (376 MHz, DMSO-d₆) δ−82.11 (s, 2F).

### Compound 2C:

R_{T}=3.550 min, mass calcd fir C₂₉H₂₇ClF₂N₆O₄ 596.2 m/z found 597.2 [M+H]⁺. Chiral HPLC (Column: Chiralpak IA 5 μm 4.6*230 nm; Mobile Phase: Hexane:EtOH=40:60 at 1.0 mL min; Temp: 30° C.; Wavelength: 254 nm, R_{T}=9.817 min). ¹HNMR (400 MHz, DMSO-d₆) δ 8.20-8.10 (m, 2H), 7.85 (br s, 2H), 7.46 (br s, 2H), 7.42-7.05 (m, 3H), 5.81-5.66 (m, 1H), 5.44-5.24 (m, 1H), 4.60-4.12 (m, 5H), 3.02-2.85 (m, 1H), 2.67-2.56 (m, 4H), 1.38-1.31 (m, 3H), 1.20-1.13 (m, 3H). ¹⁹FNMR (376 MHz, DMSO-d₆) δ−82.10 (s, 2F).

### Compound 20:

R_{T}=4.0390 min, mass calcd. for C₂₉H₂₇ClF₂N₆O₄ 596.2 m/z found 597.2 [M+H]⁺. Chiral HPLC (Column: Chiralpak IA 5 μm 4.6*250 mm; Mobile Phase: Hexane:EtOH=40:60 at 1.0 m/min; Temp: 30° C. Wavelength: 254 nm, R_{T}=12.279 min). ¹HNMR (400 MHz, DMSO-d₆) δ 8.21-8.13 (m, 1H), 7.84 (br s, 2H), 7.57 (br s, 1H), 7.41-7.38 (m, 2H), 7.20-7.01 (m, 3H), 5.79-5.68 (m, 1H), 5.44-5.25 (m, 1H), 4.96-4.10 (m, 5H), 3.01-2.84 (m, 1H), 2.50-2.39 (m, 1H), 2.18 (s, 3H), 1.57 (s, 3H), 1.22-1.12 (m, 3H). ¹⁹F NMR (376 MHz, DMSO-d₆) δ−82.32 (s, 2F).

### Compound 3A

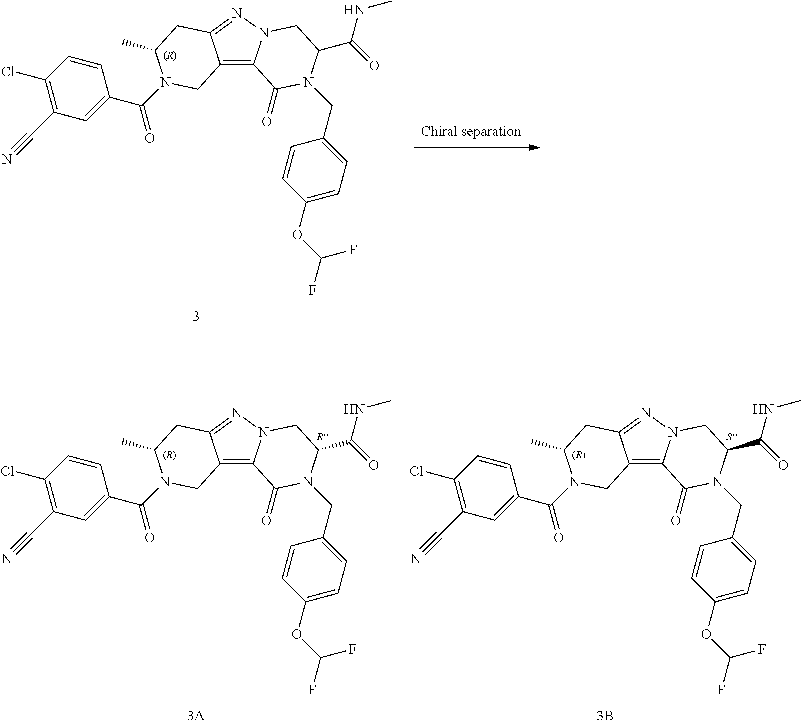

### Compound 3A:

### (3R,8R′)-2-(4-Chloro-3-cyanobenzyl)-9-(4-(difluoromethoxy)benzyl)-3-methyl-10-oxo-1,2,3,4,7,8,9,10-octahydropyrido[4′,3′:3,4]pyrazolo[1,5-a]pyrazine-8-carboxamide

Compound 3 was synthesized in an analogous way to compound 2. Compound 2 (240 mg, 100′% purity, 0.41 mmol) was separated by chiral Prep. HPLC (separation condition. Column: Chiralpak IB 5 μm 30*250 mm, Mobile Phase: Hex:EtOH=60:40 at 30 mL/min, Temp 30° C.; Wavelength: 254 nm), then further purified by Prep HPLC (Column: X-bridge C18, column sizer (5 μm 19*150 mm); Mobile Phase A: water(+0.1% ammonium bicarbonate), Mobile Phase B: acetonitrile, Gradient: 5-95% (% B), UV: 254 nm, 15 mL/min) to give compound 3A (43 mg, 0) 7% purity, 17.9% yield, 100% stereopure) as white solids and compound 3B (88 mg, 99.7% purity, 36.6% yield, 100% stereopure) as white solids.

### Compound 3A:

LC-MS (ESI): R_{T}=8.524 min, mass calcd. for C₂₈H₂₅ClF₂N₆O₄ 582.2, m/z found 583.2 [M+H]⁺. Chiral HPLC (Column: Chiralpak IB 5 μm 4.6*250 mm; Mobile Phase: Hex:EtOH=60:40) at 1 mL/min. Temp: 30° C.; Wavelength 254 nm, R_{T}=8.430 min). ¹H NMR (400 MHz, CDCl₃) δ 7.74 (s, 1H), 7.60 (s, 2H), 7.31 (d, J=8.0 Hz, 2H), 7.12 (d, J=8.4 Hz, 2H), 6.51 (t, J=73.2 Hz, 1H), 5.75-5.38 (m, 3H), 4.85 (d, J=13.2. Hz, 0.1H), 4.57-4.08 (m, 4.7H), 3.05 (br s, 1H), 2.73 (d, J=4.8 Hz, 3H), 2.69-2.65 (m, 1H), 1.31-1.20 (m, 3H). ¹⁹F NMR (376 MHz, CDCl₃) δ−8131 (s, 2F).

### Compound 3B:

LC-MS (ESI): R_{T}=8.671 min, mass calcd. for C₂₈H₂₅ClF₂N₆O₄ 582.2, m/z found 583.1 [M+H]⁺. Chiral HPLC (Column: Chiralpak IB 5 μm 4.6*250 mm, Mobile Phase: Hex:EtOH=60:40 at 1 mL/min; Temp: 30° C. Wavelength: 254 nm. R_{T}=12.580 min). ¹H NMR (400 MHz, CDCl₃) δ 7.75 (s, 1H), 7.61 (s, 2H), 7.39-7.29 (m, 2H), 7.13 (d, J=8.4 Hz, 2H), 6.51 (t, J=73.6 Hz, 1H), 5.02-5.31 (m, 3H), 4.88 (d, J=12.8 Hz, 1H), 4.67-3.89) (m, 5H), 3.02 (brs, 1H), 2.73 (d, J=4.4 Hz, 3H), 2.69-265 (m, 1H), 1.33-1.23 (m, 3H). ¹⁹F NMR (376 MHz, CDCl₃) δ−81.29 (s, 2F).

### Compound 4

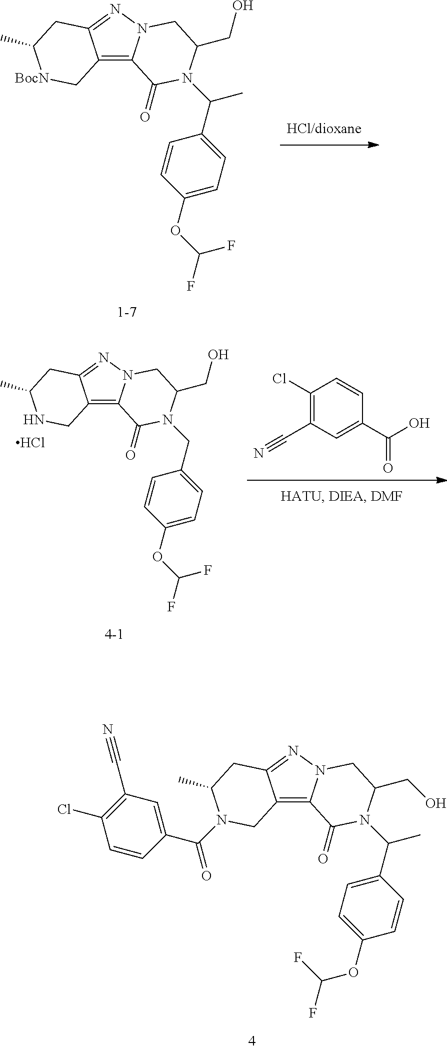 [SMILES: CC(c1ccc(OC(F)F)cc1)N1C(=O)c2c3c(nn2CC1CO)C[C@@H](C)N(C(=O)OC(C)(C)C)C3 ; C[C@@H]1Cc2nn3c(c2CN1)C(=O)N(Cc1ccc(OC(F)F)cc1)C(CO)C3 ; Cl ; N#Cc1cc(C(=O)O)ccc1Cl ; CC(c1ccc(OC(F)F)cc1)N1C(=O)c2c3c(nn2CC1CO)C[C@@H](C)N(C(=O)c1ccc(Cl)c(C#N)c1)C3]

### Intermediate 4-1:

### (3R)-9-(1-(4-(Difluoromethoxy)phenyl)ethyl)-8-(hydroxymethyl)-3-methyl-1,2,3,4,8,9-hexahydropyrido[4′,3′:3,4]pyrazolo[1,5-a]pyrazin-10(7H)-one hydrochloride

To the solution of (3R)-tert-butyl 9-(1-(4-(difluoromethoxy)phenyl)ethyl-8-(hydroxyethyl)-3-methyl-10-oxo-3,4,7,8,9,10-hexahydropyrido[4′,3′:3,4]pyrazolo[1,5-a]pyrazine-2(1H)-carboxylate 1-7 (40 mg, 100% purity, 0.079 mmol) in 1,4-dioxane (2 mL) was added 4 M hydrochloride in dioxane (0.5 mL) at 0° C. After stirred at 0° C. for 4 hours, the reaction mixture was concentrated to give the title compound (35 mg, 76% purity, 76% yield) as an oil. LC-MS (ESI): R_{T}=1.410 min, mass calcd. for C₂₀H₂₅ClF₂N₄O₃ 442.9, m/z found 407.0 [M−HCl+H]⁺.

### Compound 4:

### 2-Chloro-5-((3R)-9-(1-(4-(difluoromethoxy)phenyl)ethyl)-8-(hydroxymethyl)-3-methyl-10-oxo-1,2,3,4,7,8,9,10-octahydropyrido[4′,3′:3,4]pyrazolo[1,5-a]pyrazine-2-carboaryl)benzonitrile

A mixture of (3R)-9-(1-(4-difluoromethoxy)phenyl)ethyl)-8-(hydroxymethyl)-3-methyl-1,2,3,4,8,9-hexahydropyrido[4′,3′:3,4]pyrazolo[1,5-a]pyrazin-10(7H)-one hydrochloride 4-1 (35 ng, 76% purity, 0.06 mmol), N-ethyl-N-isopropylpropan-2-amine (49 mg, 0.379 mmol), 4-chloro-3-cyanobenzoic acid (16 ng, 0.09 mmol), 2-(31-[1,2,3]triazolo[4,5-b]pyridin-3-yl)-1,1,3,3-tetramethylisouroniumhexafluorophosphate(V) (36 mg, 0.095 mmol) in N,N-dimethylformamide (2 mL) was stirred at 30° C. for 14 hours. The mixture was acidified to pH=6 with hydrochloride aqueous solution and extracted with ethyl acetate (10 mL) twice. The combined organic layers were washed with water (10 mL) for three times and brine (10 mL), dried over Na₂SO₄₍ₛ₎, and filtered. The filtrate was concentrated under reduced pressure to give a residue, which was purified by Prep. HPLC (Column: sunfire Xbrige C18 (5 μm 19*150 mm), Mobile Phase A: Water(+0.1% NH₄HCO₃), Mobile Phase B acetonitrile, UV: 214 mm, Flow rate: 15 mL/min, Gradient: 30-60% (% B)) to get the title compound (10.1 mg, 99.5% purity, 29% yield) as white solids. LC-MS (ESI): R_{T}=3.439 min, mass calcd. for C₂₈H₂₆ClF₂N₅O₄ 569.2, m/z found 570.1 [M+H]⁺. ¹H NMR (400 M Hz, CDCl₃) δ 7.74 (s, 1H), 7.60 (s, 1H), 7.47-7.33 (m, 2H), 7.14-7.12 (m, 2H), 6.52 (t, J=73.2 Hz, 1H), 5.97 (br s, 1H), 5.71-5.42 (m, 1H), 4.88-4.18 (m, 4H), 3.87-3.78 (m, 2H), 3.58-3.47 (m, 1H), 3.09-3.04 (m, 1.6H), 2.70-2.66 (m, 1.4H), 1.66-1.65 (m, 3H), 1.30-1.27 (m, 3H) ¹⁹F NMR (376 MHz, CDCl₃) δ−81.17-−81.23 (m, 2F)

### Compound 5:

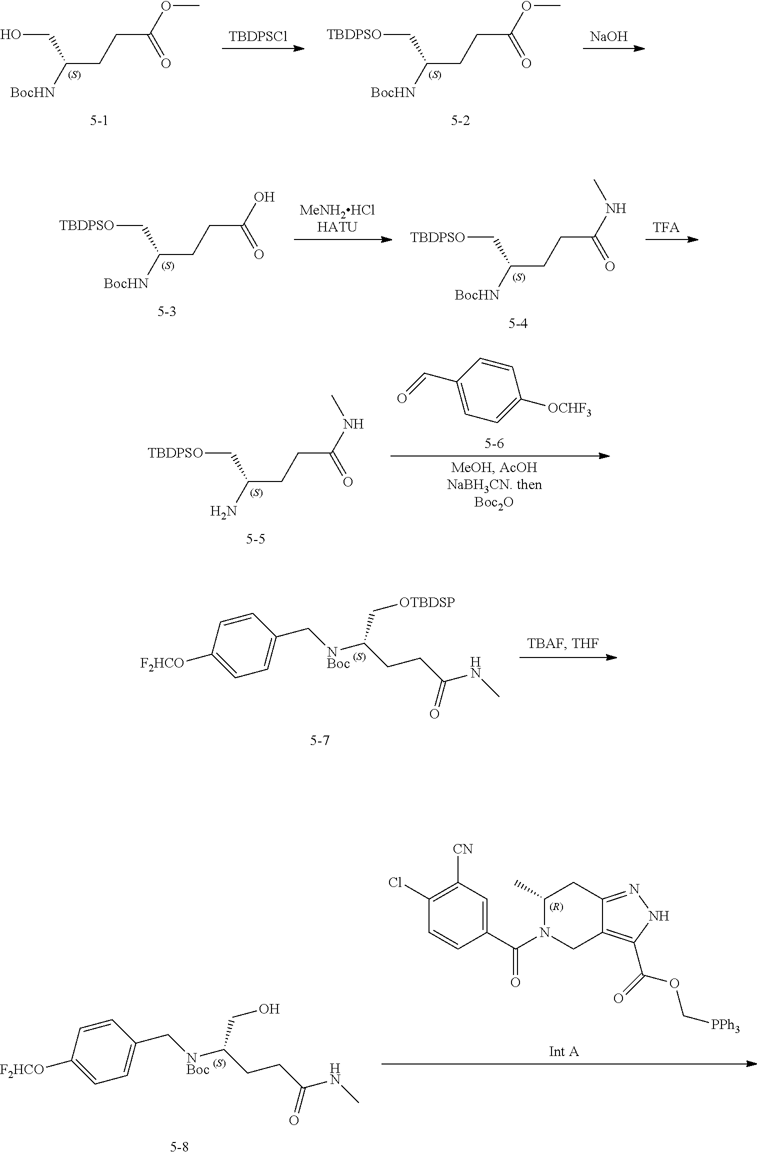

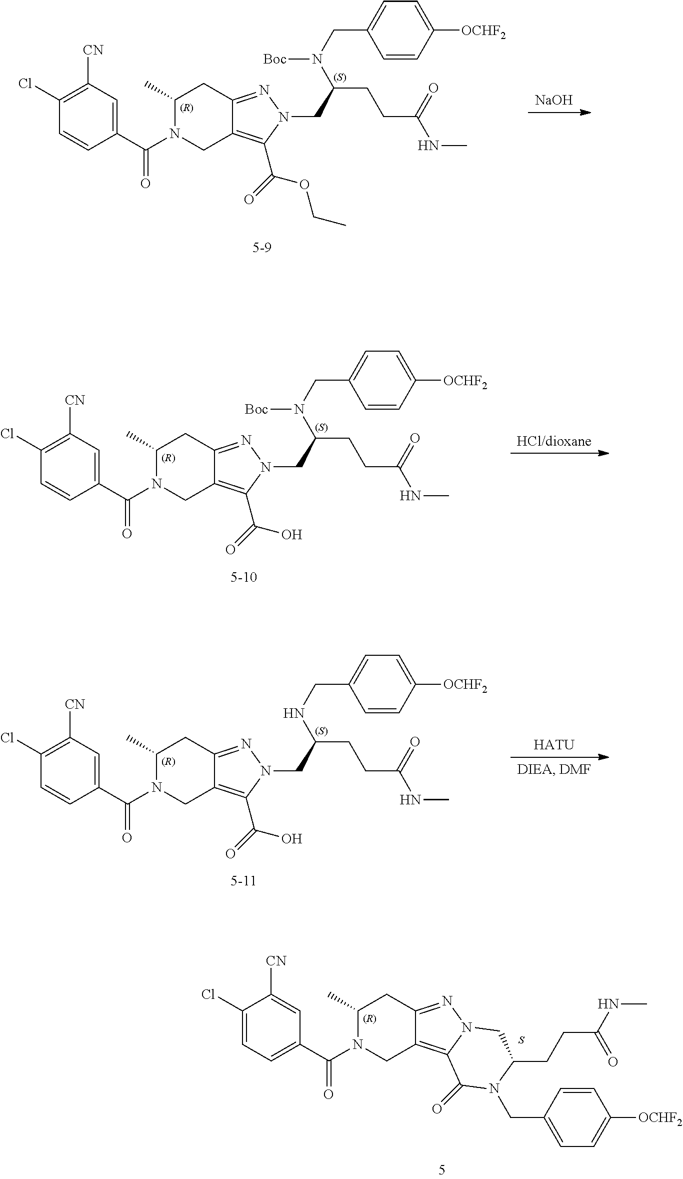

### Intermediate 5-2:

### (S)-Methyl 4-((tert-butoxycarbonyl)amino)-5-((tert-butyldiphenylsilyl)oxy)pentanoate

A solution at(S)-methyl 4-((tert-butoxycarbonyl)amino)-5-hydroxypentanoate 5-1 (1.0 g, 4.04 mmol), tert-butylchlorodiphenylsilane (1.5 g, 5.46 mmol), N-dimethylpyridin-4-amine (100 mg, 0.819 mmol) and 1H-imidazole (500 mg, 73:5 mmol) in dichloromethane (20 mL) at 0° C., After stirred at 0° C. for 4 hours, the mixture was concentrated under reduced pressure to give a crude, which was purified by C18 column (aceronitrile:water=5% to 100%) to give the title compound (2.0 g, 95% purity from HNMR, 97% yield) as white solids.

¹HNMR (409 MHz, CDCl₃) δ 7.65-7.62 (m, 4H), 7.45-7.36 (m, 6H), 4.67-4.65 (m, 1H), 3.69-3.58 (m, 6H), 2.36 (t, J=7.2 Hz, 2H), 1.94-1.79 (m, 2H), 1.43 (s, 9H), 1.07 (s, 9H).

### Intermediate 5-3:

### (S)-4-((tert-Butoxycarbonyl)amino)-5-(tert-butyldiphenylsilyl)oxy)pentanoic acid

To the solution of (S)-methyl 4-((tert-butoxycarbonyl)amino)-5-((tert-butyldiphenylsilyl)oxy)pentanoate 5-2 (1.9 g, 95% purity, 3.72 mmol) in methanol (15 mL) and water (10 mL) was added sodium hydroxide (450 mg, 11.3 mmol) under nitrogen atmosphere at 0° C. After stirred at room temperature for 2 hours, the mixture was diluted with water (30 mL), concentrated at room temperature under reduced pressure to remove the volatile. The remained aqueous layer was acidified with 1M hydrochloride aqueous solution (12 mL) and extracted with ethyl acetate (30 mL) twice and brine (60 mL), dried over Na₂SO₄₍ₛ₎ and filtered. The filtrate was concentrated to give the title compound (1.8 g, 95% purity from HNMR, 95% yield) as yellow oil.

1HNMR (400 MHz, DMSO-d₆) δ 12.0 (s, 1H), 7.63-7.61 (m, 4H) 7.48-7.38 (m, 6H), 6.61 (d, J=8.4 Hz, 1H), 3.59-3.49 (m, 3H), 2.29-2.15 (m, 2H), 1.88-1.76 (m, 1H), 1.57-1.48 (m, 1H), 1.37 (s, 9H), 0.99 (s, 9H)

### Intermediate 5-4:

### (S)-tert-Butyl (1-((tert-butyldiphenylsilyl)oxyl-5-(methylamino)-5-oxopentan-2-yl)carbamate

A solution of (S)-4-((tert-butoxycarbonyl)amino)-5-((tert-butyldiphenylsilyl)oxy)pentanoic acid 5-3 (1.8 g, 95% purity, 3.37 mmol), methylamine hydrochloride (200 mg, 6.44 mmol), N-ethyl-N-isopropylpropan-2-amine (200 mg, 6.44 mmol) and O-(7-azabenzotriazol-1-yl)-N,N,N′,N′-tetramethyluronium hexafluorophosphate (2 g, 5.26 mmol) in N,N-dimethylformamide (10 mL) at 0° C. After stirred at 330° C. for 16 hours, the mixture was concentrated under reduced pressure to give a residue. The crude residue was purified by CIS column (acetonitrile:water=5% to 100%) to give the title compound (1.5 g, 95% purity from HNMR, 87% yield) as white solids.

¹HNMR (400 MHz, CDCl₃) δ 7.65-7.63 (m, 4H), 7.47-7.38 (m, 6H), 6.47 (br s, 1H), 4.84-4.82 (m, 1H), 3.71-3.56 (m, 3H), 2.80 (d, J=4.8 Hz, 3H), 2.27-2.15 (m, 2H), 1.87-1.82 (m, 2H), 1.46 (s, 9H), 1.07 (s, 9H)

### Intermediate 5-5:

### (S)-4-Amino-5-(tert-butyidiphenylsilyl)oxy)-N-methylpentanamide hydrochloride

A solution of (S)-tert-butyl (1-((tert-butyldiphenylsilyl)oxy)-5-(methylamino)-5-oxopentan-2-yl)carbamate 5-4 (1.57 g, 3.08 mmol) in dichloromethane (10 mL) was added 2,2,2-trifluoroacetic acid (5 mL) stirred at room temperature for 2 hours. The mixture was concentrated under vacuum to give a crude. Then the mixture was adjusted to pH˜8 with sodium bicarbonate aqueous solution, extracted with dichloromethane (50 ml) twice. The combined organic layers were washed with brine (100 mL), dried over Na₂SO₄₍ₛ₎, and filtered. The filtrate was concentrated to give the tide compound (1.2 g, 95% purity from HNMR, 96% yield) as a light yellow oil.

¹HNMR (400 MHz, CDCl₃) δ 7.64-7.63 (m, 4H), 7.49-7.45 (m, 6H), 6.9 (br s, 2H), 3.68-3.56 (m, 2H), 3.17-3.12 (m, 1H), 2.55 (d, J=4.4 Hz, 3H), 2.16 (t, J=7.2 Hz, 2H), 1.87-1.66 (m, 2H), 1.02 (s, 9H).

### Intermediate-5-7:

### (S)-tert-Butyl (1-((tert-butyldiphenylsilyl)oxy)-5-(methylamino)-5-oxopentan-2-yl)(4-(difluoromethoxy)benzyl)carbamate

A mixture of 4-amino-5-((tert-butyldiphenylsilyl)oxy)-N-methylpentanimidamide 5-5 (1.2 g, 95% purity, 2.96 mmol) and 4-(difluoromethoxy)benzaldehyde 5-61.5 g, 8.71 mmol) in methanol (20 mL) was added acetic acid (0.5 mL). After stirred at 30° C. for 2 hours, the reaction mixture was cooled to 0° C., followed by addition of sodium cyanoborohydride (500 mg, 7.96 mmol) and stirred at room temperature for 1 hour. The reaction was quenched with water (5 mL), and filtered through a pad of celite. The tilter cake was washed with ethyl acetate (30 mL). The filtrate was concentrated to give a residue. The crude product in tetrahydrofuran (10 mL) and water (10 ml) was added triethylamine (500 mg, 4.94 mmol) and di-tert-butyl dicarbonate (1.0 g, 4.58 mmol) at 0° C. After stirred at 30° C. for 1 hour, the reaction mixture was concentrated under reduced pressure to give a residue, which was purified by C18 column (acetonitrile:water=5% to 100%) to give the title compound (1:3 g, 80% purity, 75% yield) as a yellow oil.

R_{T}=1.94 min, mass calcd. for C₃₅H₄₆F₂N₂O₅Si 640.3 m/z found 640.9 [M+H]⁺

### Intermediate 5-8:

### (S)-tert-Butyl 4-(difluoromethoxy)benzyl(I-hydroxy-5-(methylamino)-5-oxopentan-2-yl)carbamate

The (S)-tert-butyl (1-((tert-butyldiphenylsilyl)oxy)-5-(methylamino)-5-oxopentan-2-yl)(4-(difluoromethoxy)benzyl)carbamate 5-7 (1.5 g, 80% purity, 1.87 mmol) in tetrahydrofuran (10 mL) was added 1 M tetrabutylammonium fluoride in tetrahydrofuran (2 mL, 2 mmol) at 0° C. After stirred at 0° C. for 4 hours, the mixture was filtered and concentrated under reduced pressure to give a residue. The crude was purified by C18 column (acetonitrile:water=5% to 100%) to give the title compound (800 mg, 0% purity from HNMR, 96% yield) as yellow oil.

¹HNMR (400 MHz, CDCl₃) δ 7.31-7.29 (m, 2H), 7.09-7.07 (m, 2H), 6.50 (t, J=74.0 Hz, 1H), 5.74-5.24 (m, 1H), 4.51-4.30 (m, 2H), 3.85-3.57 (m, 3H), 3.36-3.19 (m, 1H), 2.77 (d, J=4.8 Hz, 3H), 2.17-210 (m, 1.5H), 1.99-1.85 (m, 2.5H), 1.43 (s, 9H).

### Intermediate 5-9:

### (R)-Ethyl 2-((S)-2-((tert-butoxycarbonyl)(4-(difluoromethoxy)benzyl)amino)-5-(methylamino)-5-oxopentyl)-5-(4-chlor-3-cyanobenzoyl)-6-methyl-4,5,7-tetrahydro-2H-pyrazolo[4,3-c]pyridine-3-carboxylate

To a solution of (R)-ethyl 5-(4-chloro-3-cyanobenzoyl)-6-methyl-4,5,6,7-tetrahydro-2H-pyrazolo[4,3-c]pyridine-3-carboxylate Int A (500 ng, 95% purity, 1.27 mmol), (S)-tert-butyl 4-difluoromethoxy)benzyl(1-hydroxy-5-(methylamino)-5-oxopentan-2-yl)carbamate 5-8 (700 mg, 1.74 mmol) and triphenylphosphine (700 mg, 2.67 mmol) in tetrahydrofuran (10 mL) was added di-ten-butyl diazene-1,2-dicarboxylate (600 mg, 2.61 mmol) at 0° C. After stirred at 50° C. for 4 hours, the mixture was concentrated under reduced pressure to give a crude, which was purified by C18 column (acetonitrile:water:=5% to 100%) to give the title compound (550 mg, 101% purity, 57% yield) as white solids. LC-MS (ESI): R_{T}=1.67 min and 1.70 min, mass calcd. for C₃₇H₄₃ClF₂N₆O₇ 756.3 found 756.9 [M+H]⁺.

### Intermediate 5-10:

### (R)-2-((S)-2-(tert-Butoxycarbonyl)(4-(difluoromethoxy)benzyl)amino)-5-(methylamino)-5-oxopentyl)-5-(4-chloro-3-cyanobenzoyl)-6-methyl-4,5,6,7-tetrahydro-2H-pyrazolo[4,3-c]pyridine-3-carboxylic acid

To the solution of ((R)-ethyl 2-((S)-2-((tert-butoxycarbonyl)(4-(difluoromethoxy)benzyl)amino)-5-(methylamino)-5-oxopentyl)-5-(4-chloro-3-cyanobenzoyl)-6-methyl-4,5,6,7-tetrahydro-2H-pyrazolo[4,3-c]pyridine-3-carboxylate 5-9 (500 mg, 100% purity, 0.66 mmol) in methanol (5 mL) and water (5 mL) as added sodium hydroxide (100 mg, 2.50 mmol) under nitrogen atmosphere at 0° C. After stirred at room temperature. For 2 hours, the mixture was diluted with water (50 mL), concentrated under reduced pressure to remove the volatile. The remained aqueous layer was acidified with 1M hydrochloride aqueous solution (3 mL), extracted with ethyl acetate (40 mL) twice, washed with brine (100 mL), dried over Na₂SO₄₍ₛ₎ and filtered. The filtrate was concentrated to give the title compound (400 ng, 97% purity, 81% yield) as yellow oil. LC-MS (ESI): R_{T}=1.37 min. mass calcd. for C₃₅H₂₉ClF₂N₆O₇ 728.3 found 728.8 [M+H]⁺.

### Intermediate 5-11:

### (R)-5-(4-Chloro-3-cyanobenzoyl)-2-((S)-2-((4-(difluoromethoxy)benzyl)amino)-5-(methylamino)-5-oxopentyl)-6-methyl-4,5,6,7-tetrahydro-2H-pyrazolo[4,3-c]pyridine-3-carboxylic acid

A solution of (R)-2-((S)-2-((tert-butoxycarbonyl)(4-difluoromethoxy)benzyl)amino)-5-(methylamino)-5-oxopentyl 5-t 4-chloro-3-cyanobenzoyl-6-methyl-4,5,6,7-tetrahydro-2H-pyrazolo[4,3-c]pyridine-3-carboxylic acid 5-10 (400 mg, 97% purity, 0.532 mmol) in 4 M hydrochloride in 1,4-dioxane (4 mL) was stirred at 0° C. for 1 hour. The mixture was concentrated under reduced pressure to give the title compound (330 mg, 94% purity, 88% yield) as white solids. LC-MS (ESI): R_{T}=1.31 min, mass calcd. for C₃₀H₃₁ClF₂N₆O₅ 628.2 found 628.9 [M+H]⁺.

### Compound 5:

### 3-((3R,8S)-2-(4-chloro-3-cyanobenzoyl)-9-(4-(difluoromethoxy)benzyl)-3-methyl-10-oxo-1,2,3,4,7,8,9,10-octahydropyrido[4′3′:3,4]pyrazolo[1,5-a]pyrazin-8-yl)-N-methylpropanamide

To a solution of (R)-5-(4-chloro-3-cyanobenzoyl-2-((S)-2-((4-(difluoromethoxy)benzyl)amino)-5-(methylamino)-5-oxopentyl)-4-methyl-4,5,6,7-terrahydro-2H-pyrazolo[4,3-c]pyridine-3-carboxylic acid 5-11 (330 mg, 04% purity, 0.466 mmol), N-ethyl-N-isopropylpropan-2-anine (400 mg, 3.10 mmol) in N,N-dimethylformamide (10 mL) was added O-(7-azabenzotriazol-1-yl)-N,N,N′,N′-tetramethyluronium hexafluorophosphate (280 mg, 0.736 mmol) at 0° C. After stirred at 30° C. for 16 hours, the mixture was concentrated under reduced pressure to give a residue, which was purified by C18 column (acetonitrile:water(+0.02% ammonium acetate)=5% to 100%) to give the title compound (150 mg, 08.3% purity, (0) % yield) as while solids. LC-MS(ESI) R_{T}=0.291 min, mass calcd. for C₃₀H₂₉ClF₂N₆O₄ 610.2 found 611.2 [M+H]⁺. ¹H NMR (400 MHz, CDCl₃) δ 7.74 (s, 1H), 7.62-7.51 (m, 2H), 7.36-7.34 (m, 2H), 7.08 (d, J=8.4 Hz, 2H), 6.49 (t, J=73.6 Hz, 1H), 5.79-5.39 (m, 3H), 4.85-3.81 (m, 6H) 3.15-2.99 (m, 1H), 2.83-2.65 (m, 4H), 2.31-2.23 (m, 1H), 2.15-2.03 (m, 2), 1.85-1.76 (m, 1H), 1.28-1.26 (m, 3H).

¹⁹F NMR (376 MHz, CDCl₃) δ−80.98 (s, 2F).

### Compound 6

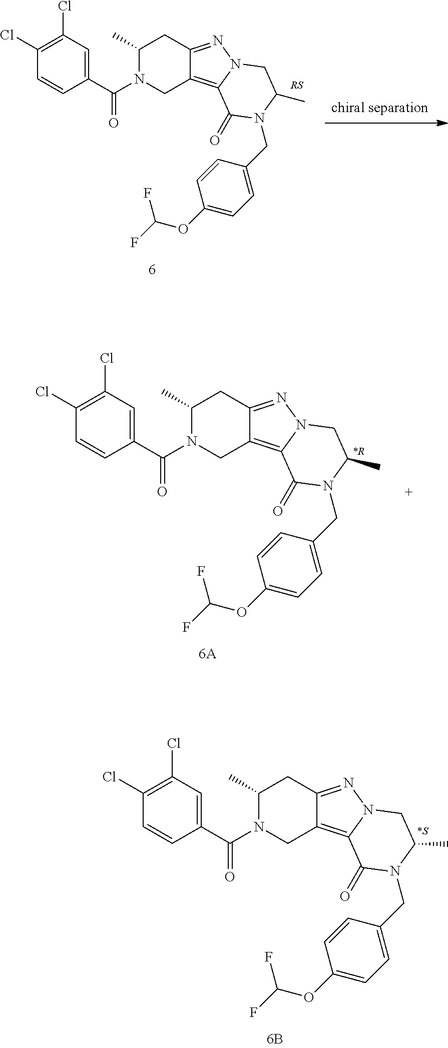

### Compounds 6A and 6B:

### (3R,8R*)-2-(3,4-dichlorobenzoyl)-9-(4-(difluoromethoxy)benzyl)-3,8-dimethyl-1,2,3,4,8,9-hexahydropyrido[4′,3′:3,4]pyrazolo[1,5-a]pyrazin-10(7H)-one (6A) and (3R,8S*)-2-(3,4-dichlorobenzoyl)-9-(4-(difluoromethoxy)benzyl)-3,8-dimethyl-1,2,3,4,8,9-hexahydropyrido[4′,3′:3,4]pyrazolo[1,5-a]pyrazin-10(7H)-one (6B)

(3R)-2-(3,4-dichlorobenzoyl)-9-(4-(difluoromethoxy)benzyl)-3,8-dimethyl-1,2,4,8,9-hexahydropyrido[4′,3′:3,4]pyrazolo[1,5-a]pyrazin-10(7H)-one compound 6 (160 mg, 100% purity, 0.201 mmol) was synthesized in an analogous way to compound 2. Compound 6 was separated by chiral prep HPLC (separation method: Column: Chiralpak IA 5 μm 30*250 mm; Mobile Phase: CO₂:EtOH (0.2% DEA)=60:40 at 60 g/min; Col. Temp: 40° C.; Wavelength: 214 nm, Back pressure: 100 bar), then further purification by C18 (acetonitrile:water(+0.02% ammonium acetate)=5% to 95%) to give compound 6A (45.6 mg, 99.8% purity, 28% yield, 100% stereopure) as white solids and compound 6B (38.1 mg, 99.5% purity, 24% yield, 100% stereopure) as white solids.

### Compound 6A:

LC-MS (ESI): R_{T}=7.544 min, mass calcd. for C₁₆H₂₄Cl₂F₂N₄O₃ 548.1, m/z found 549.1 [M+H]⁺. Chiral analysis (Column: Superchiral 1A 5 μm 4.6*250 mm; Mobile Phase: CO₂:EtOH (0.2% DEA)=60:40 at 1.20 mL/min; Temp: 40° C.; Wavelength: 254 nm, Back pressure: 100 bar. R_{T}=7.36 min). ¹H NMR (400 MHz, CDCl₃) δ 7.54 (d, J=2.0 Hz, 1H), 7.51 (d, J=8.0 Hz, 1H), 7.36-7.26 (m, 3H), 7.11 (d, J=8.8 Hz, 2H), 6.50 (t, J=73.6 Hz, 1H), 5.87-5.41 (m, 1H), 5.37 (d, J=15.2 Hz, 1H), 4.83-4.36 (m, 2H), 4.27 (dd, J=13.6, 4.4 Hz, 1H), 4.13 (d, J=13.2 Hz, 1H), 4.08-3.86 (m, 1H), 3.82-3.74 (m, 1H), 3.18-2.93 (m, 1H), 2.77-2.60 (m, 1H), 1.27 (d, J=6.4 Hz, 6H) ¹⁹F NMR (376 MHz, CDCl₃) δ−81.03 (s, 2F).

### Compound 6B:

LC-MS (ESI): R_{T}=7.587 min, mass calcd. for C₂₆H₂₄Cl₂F₂N₄O₃ 548.1, m/z found 549.1 [M+H]⁺. Chiral analysis (Column: Superchiral IA 5 μm 4.6*250 mm; Mobile Phase, CO₂:EtOH (0.2% DEA) 60:40 at 1.20 mL/min; Temp 40° C.; Wavelength: 254 nm, Back pressure: 10) bar; R_{T}=9.57 min). ¹H NMR (400 MHz, CDCl₃) 7.54 (d, J=2.0 Hz, 1H), 7.51 (d, J=8.0 Hz, 1H), 7.34-7.25 (m, 3H), 7.10 (d, J=8.0 Hz, 2H), 6.50 (t, J=73.2 Hz, 1H), 5.06-5.20 (m, 2H), 5.02-4.32 (m, 2R), 4.27 (dd, J=12.8, 4.4 Hz, 1H), 4.13 (dd, J=13.2, 2.4 Hz, 1H), 4.02 (d. J=14.8, 1H), 3.86-3.76 (m, 1H), 3.21-2.95 (m, 1H), 2.67 (d, J=15.6 Hz, 1H) 1.36-1.20 (m, 61H) ¹⁹F NMR (376 MH, CDCl₃) δ−81.03 (s, 2F).

### Compound 7:

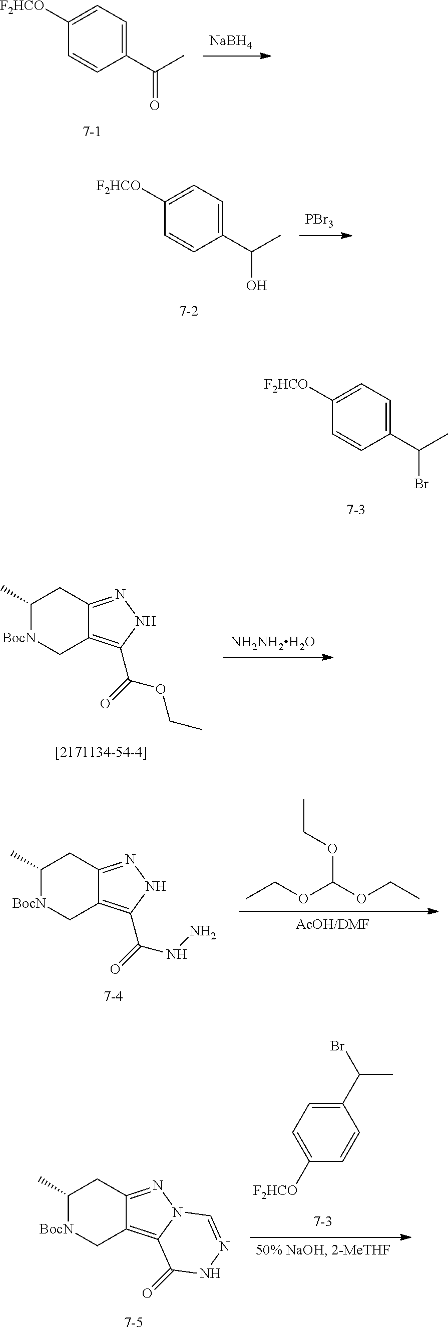

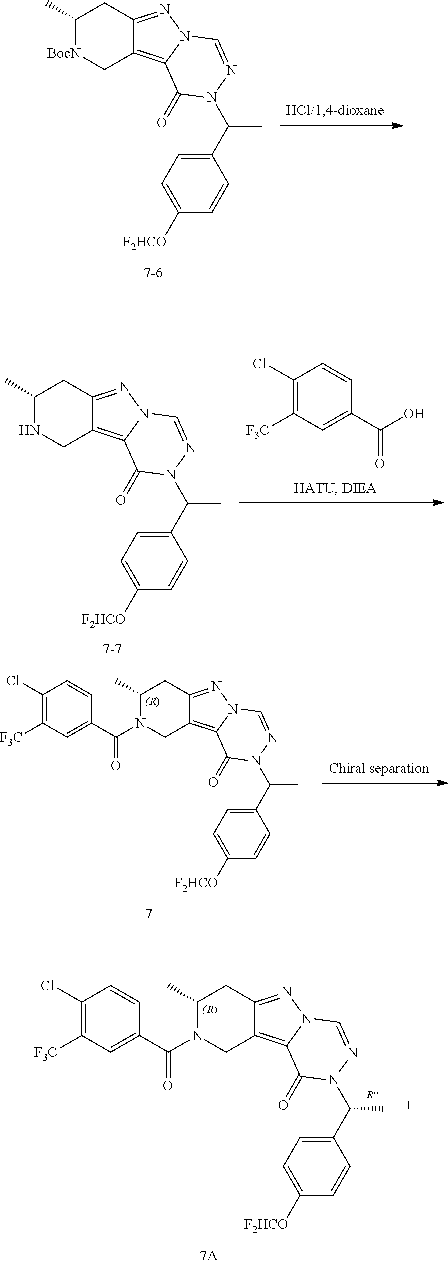

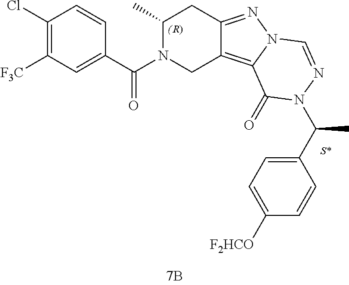

### Intermediate 7-2:

### 1-(4-(Difluoromethoxy)phenyl)ethanol

To a solution of 1-(4-(difluoromethoxy)phenyl)ethanone 7-1 (50 g, 26.9 mmol) in methanol (50 mL) was slowly added sodium borohydride (3.0 g, 79.3 mmol) at 0° C. After stirred at room temperature, for 3 hours, the reaction was quenched with acetone (50 mL) dropwise and concentrated to give a residue. The residue was dissolved into ethyl acetate (50 mL), washed with brine (50 mL) twice, dried over Na₂SO₄₍ₛ₎ and filtered. The filtrate was concentrated to give the tithe compound (5.3 g, 90% purity front HNMR, 94% yield) as colourless oil. ¹H NMR (400 MHz, CDCl₃) δ δ 7.38 (d, J=8.4 Hz, 2H), 7.10 (d, J=0.4 Hz, 1H), 6.50 (t, J=74.0 Hz, 1H), 4.91 (q, J=6.0 Hz, 1H), 180 (d, J=3.6 Hz, 1H), 1.49 (d, J=6.4 Hz, 3H)

### Intermediate 7-3:

### 1-(1-Bromoethyl)-4-(difluoromethoxy)benzene

To a solution of 1-(4-(difluoromethoxy)phenyl)ethanol 7-2 (500 ng 0% purity, 2.39 mmol) in dichloromethane (5 mL) was slowly added phosphorus(II) bromide (650 mg, 2.40 mmol) at 0° C. After stirred at room temperature for 2 hours, the reaction mixture was poured into ice water (20 mL) and extracted with dichloromethane (30 mL) twice. The combined organic layers were washed with saturated sodium bicarbonate solution (50 mL×2), brine (50 mL×2), dried over Na₂SO₄₍ₛ₎ and filtered. The filtrate was concentrated under reduced pressure to give the title compound (59) mg, 95% purity from HNMR, 93% yield) as colourless oil ¹H NMR (400 MHz, CDCl₃) 7.44 (d, J=8.4 Hz, 2H), 7.09 (d, J=8.8 Hz, 2H), 6.51 (t, J=73.6 Hz, 1H), 5.20 (q, J=7.2 Hz, 1H), 2.03 (d, J=6.8 Hz, 3H).

### Intermediate 7-4:

### (R)-tert-Butyl-3-(hydrazinecarbonyl)-6-methyl-6,7-dihydro-2H-pyrazolo[4,3-c]pyridine-5(4H)-carboxylate

To a solution of (R)-5-tert-buty 3-ethyl 6-methyl-6,7-dihydro-2H-pyrazolo[4,3-c]pyridine-3,5(4H)-dicarboxylate (2.0 g, 92% purity, 6.47 mmol) in ethanol (20 mL) was added hydrazine monohydrate (4 mL) at room temperature. After stirred at 85° C. for 16 hours, the mixture was concentrated under reduced pressure. The resulting residue was dissolved with ethyl acetate (40 nm), washed with brine (20 mL), and dried over Na₂SO₄₍ₛ₎ The organic layer was concentrated to give the desired product (2.0 g, 42% purity, 100% yield) as white solid. LC-MS (ESI): RT=1.36 min, mass calcd. for C₁₃H₂₁N₅O₃ 295.2, m/z found 296.2 [M+H]⁺.

### Intermediate 7-5:

### (R)-tert-Butyl-8-methyl-1-oxo-1,2,7,8-Tetrahydropyrido[4′,3′:3,4]pyrazolo[1,5-d][1.2.4]triazine-9(10H)-carboxylate

To a solution of (R)-tert-butyl 3-(hydrazinecarbonyl)-6-methyl-6,7-dihydro-2H-pyrazolo[4,3-c]pyridine-5(41)-carboxylate 7-4 (2.0 g, 92% purity, 6.23 mmol) in N,N-dimethylformamide (20 mL) and acetic acid 05 mL) was added triethoxymethane (6.69 g, 45.1 mmol) at room temperature. After stirred at 80° C. for 16 hours, the mixture was diluted with saturated sodium carbonate solution (50 ml) and extracted with ethyl acetate (100 mL). The organic layer was washed with brine (80 ml) for three times, dried over Na₂SO₄₍ₛ₎ and concentrated under vacuum to give a residue. The crude product was purified by silica gel column chromatography (petroleum ether ethyl acetate=5:1 to 3:1) to give the title compound (1.3 g, 95% purity from HNMR, 98% yield) as white solids ¹H NMR (400 MHz, CDCl₃) δ 9.53 (br s, 1H), 8.32 (s, 1H), 5.31-5.23 (m, 1H), 5.03-4.91 (m, 1H), 4.47 (d, J=18.0 Hz, 1H), 3.10 (dd, J=16.4 and 6.4 Hz, 1H), 2.77 (d, J=16.4 Hz, 1H), 1.50 (s, 9H), 1.14 (d, J=6.8 Hz, 3H).

### Intermediate 7-6:

### (8R)-tert-Butyl 2-(1-(4-(difluoromethoxy)phenyl)ethyl)-8-methyl-1-oxo-1,2,7,8-tetrahydropyrido[4′3′:3,4]pyrazolo[1,5-d][1,2,4]triazine-9(10H)-carboxylate

To a solution of (R)-tert-butyl 8-methyl-1-oxo-1,2,7,8-tetrahydropyrido[4′,3′:3,4]pyrazolo[1,5-d][1,2,4]triazine-9(10H)-carboxylate 7-5 (100 mg, 95% purity, 0.311 mmol), 1-(1-bromoethyl)-4-(difluoromethoxy)benzene 7-3 (100 mg, 95% purity, 0.378 mmol) in 2-methyltetrahydrofuran (1 mL) was added 50% sodium hydroxide aqueous solution (1 mL) at rt. After stirred at 50° C. for 2 hours, the reaction mixture was quenched with ice water (10 mL) and extracted with ethyl acetate (10 mL) twice. The combined organic layers were washed with brine (20 mL) twice, dried over Na₂S₄₍ₛ₎ and filtered. The filtrate was concentrated under reduced pressure to give a crude, which was purified by C18 column (acetonitrile:water=60% to 80%) to give the title compound (160 mg, 90% purity from HNMR, 97% yield) as colorless oil ¹H NMR (400 MHz, CDCl₃) δ 8.32 (s, 1H), 7.48 (t, J=8.8 Hz, 2H), 7.09 (d, J=8.4 Hz, 2H), 6.48 (t, J=73.6 Hz, 1H), 6.21 (q, J=7.2 Hz, 1H), 5.30-5.25 (m, 1H), 4.99-4.90 (m, 1H), 4.39-4.32 (m, 1H), 3.10-3.05 (m, 1H), 2.75-2.71 (m, 1H), 1.77 (d, J=7.2 Hz, 3H), 1.49 (d, J=0.01 Hz, 9H), 1.13-1.09 (m, 3H)

### Intermediate 7-7:

### (8R)-2-(1-(4-(Difluoromethoxy)phenyl)ethyl)-8-methyl-7,8,9,10-tetrahydropyrido[4′,3′:3,4]pyrazolo[1,5-d][1,2,4]triazin-1(2H)-one hydrochloride

To a solution of (8R)-tert-butyl 2-(1-(4-(difluoromethoxy)phenyl)ethyl)-8-methyl-1-oxo-1,2,7,8-tetrahydropyrido[4′3′:3,4]pyrazolo[1,5-d][1,2,4]triazine-9(10H)-carboxylate 7-6 (160 mg, 90% purity, 0.303 mmol) in dichloromethane (5 mL) was added 2 M hydrochloride in 14-dioxane (2.0 mL 4.0 mmol) at 0° C. After stirred at room temperature for 3 hours, the mixture was concentrated under reduced pressure to give title compound (145 mg, 80% purity from HNMR, 93% yield) as white solids. ¹H NMR (400 MHz, CDCl₃) δ 9.69 (br s, 2H), 9.06 (s, 1H), 7.44-7.41 (m, 2H), 7.21 (d, J=73.6 Hz, 1H), 7.14 (d, J=8.4 Hz, 2H), 6.09 (q, J=5.6 Hz, 1H), 4.53-4.42 (m, 2H), 3.71-3.65 (m, 1H), 3.23-3.19 (m, 1H), 2.94-2.87 (m, 1H), 1.70 (d, J=6.8 Hz, 3H), 1.44-1.42 (m, 3H).

### Compound 7:

### (8R)-9-Chloro-3-(trifluoromethyl)benzoyl)-2-(1-(4-(difluoromethoxy)phenyl)ethyl)-8-methyl-7,8,9,10-tetrahydropyrido[4′,3′:3,4]pyrazolo[1,5-d][1,2,4]triazin-1(2H)-one

To a solution of (8R)-2-(1-(4-(difluoromethoxy)phenyl)ethyl)-8-methyl-7,8,9,10-tera hydropyrido[4′,3′:3,4]pyrazolo[1,5-d][1,2,4]triazin-1(2H)-one hydrochloride 7-7 (145 mg, 80% purity, 0.282 mmol), 4-chloro-(trifluormethyl)benzoic acid (100 mg, 0.445 mmol) and N,N-diisopropylethylamine (0.3 mL, 1.82 mmol) in N,N-dimethylformamide (5 mL) was added 1-[bis(dimethylamino)methylene]-1H-1,2,3-triazolo[4,5-b]pyridinium-3-oxide hexafluorophosphate (200 ng, 0.526 mmol) at 0° C. After stirred at room temperature for 16 hours, the reaction mixture was quenched with water (50 mL) and extracted with ethyl acetate (20 mL) twice. The combined organic layers were washed with brine (50 mL) twice, dried over Na₂SO₄₍ₛ₎ and filtered. The filtrate was concentrated under reduced pressure to give the title compound (180 mg, 89% purity from LCMS, 98% yield) as white solids LC-MS (ESI): R_{T}=1.74 min, mass calcd. for C₂₆H₂₇ClF₅N₅O₃ 581.1, m/z found 582.2 [M+H]⁺.

### Compounds 7A and 7B:

### (R)-9-(4-Chloro-3-(trifluoromethyl)benzoyl)-2-((R*)-1-(4-(difluoromethoxy)phenyl)ethyl)-8-methyl-7,8,9,10-tetrahydropyrido[4′,3′:3,4]pyrazolo[1,5-d][1,2,4]triazin-1(2H)-one (7A) and

### (R)-9-(4-chloro-3-(trifluoromethyl)benzoyl)-2-((S*)-1-(4-(difluoromethoxy)phenyl)ethyl)methyl-7,8,9,10-tetrahydropyrido[4′,3′:3,4]pyrazolo[1,5-d][1,2,4]triazin-1(2H)-one (7B)

The racemic of (8R)-9-(4-chloro-3-(trifluoromethyl)benzoyl)-2-(1-(4-(difluoromethoxy)phenyl)ethyl)-8-methyl-7,8,9,10-tetrahydropyrido[4′,3′:3,4]pyrazolo[1,5-d][1,2,4]triazin-1(2H)-one 7 (180 mg, 89′% purity, 0.275 mmol) was separated by chiral Prep. HPLC (separation method: Column: Chiralpak IC 5 μM 30*250 mm, Mobile Phase: Hex:EtOH=50:50 at 30 mL/min; Col Temp: 40° C.; Wavelength: 214 nm, Back pressure: 100 bar) to give compound 7A (40.0 mg, 99.9% purity, 25% yield, 100% stereopure) as white solids and compound 7B (50.2 mg, 99.7% purity, 31% yield, 99.49% stereopure) as white solids.

### Compound 7A:

LC-MS (ESI): R_{T}=8.944 min mass calcd. for C₂₆H₂₁ClF₅N₅O₅ 581.1, m/z found 582.1 [M+H]⁺. Chiral analysis (Column: Superchiral IC 5 μm 4.6*250 mm; Mobile Phase, Hex:EtOH=50:50 at 1.00 mL/min; Temp: 30° C., Wavelength: 254 nm, Back pressure: 98 bar; Rt=6.729 min). ¹H NMR (400 MHz, DMSO-d₄) δ 9.00 (br s, 1H), 7.95 (s, 1H), 7.86-7.81 (m, 2H), 7.49-7.34 (m, 2.2H), 7.20-7.01 (m, 2.8H), 6.21-5.96 (m, 1H), 5.66-5.25 (m, 1H), 4.85-4.18 (m, 2H) 3.21-3.02 (m, 1H), 2.91-2.64 (m, 3H), 1.69 (s, 3H), 1.32-1.06 (m, 3H).

¹⁹F NMR (376 MHz, DMSO-d₆) δ−61.33 (s, 3F), −81.03 (s, 2F).

### Compound 7B:

LC-MS (ESI): R_{T}=9.105 min, mass calcd. for C₂₆H₂₁ClF₅N₅O₃ 581, m/z found 582.1 [M+H]⁺. Chiral analysis (Column: Superchiral IC 5 μm 4.6*250 mm; Mobile Phase: Hex:EtOH=50:50 at 1.00 mL/min, Temp: 30° C., Wavelength: 254 nm, Back pressure: Q8 bar, Rt=8.213 min). ¹H NMR (400 MHz, DMSO-d₆) δ 9.01 (br s, 1H), 7.96 (s, 11H), 7.87-7.82 (m, 2H), 7.50-7.44 (m, 2H), 7.21 (t, J=74.4 Hz, 1H), 7.14 (d, J=7.6 Hz, 2H) 6.22-5.92 (m, 1H), 5.67-5.20 (m, 1H), 4.84-4.12 (m, 2H), 3.23-3.03 (m, 1H), 2.89-2.63 (m, 1H), 1.69 (s, 3H), 1.32-1.01 (m, 3H).

¹⁹F NMR (376 MHz, DMSO-d₆) δ−61.31 (s, 3F), −81.95 (N, 2F).

### Compound 8A

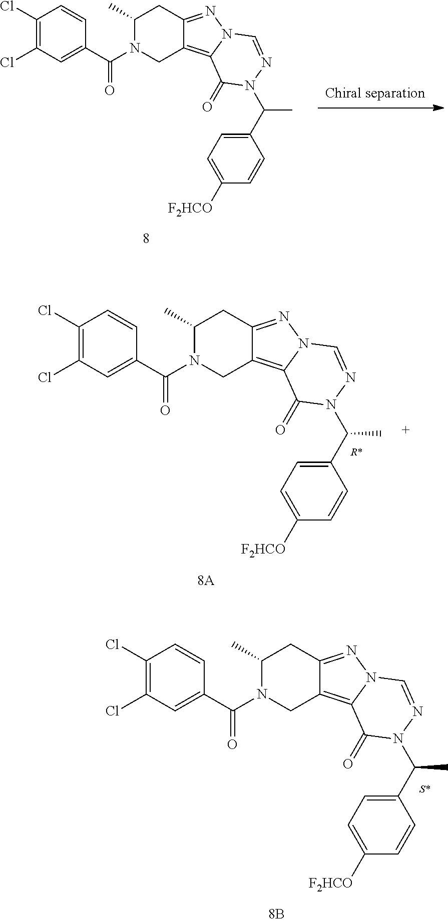 [SMILES: CC(c1ccc(OC(F)F)cc1)n1ncn2nc3c(c2c1=O)CN(C(=O)c1ccc(Cl)c(Cl)c1)[C@H](C)C3 ; "Chiral separation" ; C[C@@H]1Cc2nn3cnn([C@H](C)c4ccc(OC(F)F)cc4)c(=O)c3c2CN1C(=O)c1ccc(Cl)c(Cl)c1 ; C[C@@H]1Cc2nn3cnn([C@@H](C)c4ccc(OC(F)F)cc4)c(=O)c3c2CN1C(=O)c1ccc(Cl)c(Cl)c1]

### Compounds 8A and 8B:

### (R)-9-(3,4-Dichlorobenzoyl)-2-((R*)-1-(4-(difluoromethoxy)phenyl)ethyl)-8-methyl-7,8,9,10-tetrahydropyrido[4′,3′:3,4]pyrazolo[1,5-d][1,2,4]triazin-1(2H)-one (8A) and

### (R)-9-(3,4-dichlorobenzoyl)-2-((S*)-t-(4-(difluoromethoxy)phenyl)ethyl)-8-methyl-7,8,9,10-tetrahydropyrido[4′,3′:3,4]pyrazolo[1,5-d][1,2,4]triazin-1(2H)-one (8B)

(8R)-9-(3,4-dichlorobenzoyl)-2-(1-(4-(difluoromethoxy)phenyl)ethyl)-8-methyl-7,8,9,10-tetrahydropyrido[4′,3′:3,4]pyrazolo[1,5-d][1,2,4]triazin-1(2H)-one 8 (410 mg, 95% purity, 0.710 mmol) was synthesized in an analogous way to compound 7. Compound 8 separated by chiral HPLC (separation condition: Column: Chiralpak IC 5 μm 30*250 mm. Hex:EtOH=40:60, 30 mil/in, 254 nm, Col. Temp: 30° C.), further purified by C18 column (acetonitrile:water(+0.02% ammonium acetate)=5% to 100%) to give compound 8A (120 mg, 99.8% purity, 70% yield, 100% stereopure) and compound 8B (150 mg, 99.9% purity, 78.9% yield, 99.9% stereopure) as while solids.

### Compound 8A:

LC-MS (ESI): R_{T}=4.167 min, mass calcd. for C₂₅H₂₁Cl₂F₂N₅O₅ 547.1, m/z found 548.1 [M+H]⁺. Chiral analysis (Column: Chiralpak KC 5 μm 4.6*250 mm; Mobile Phase: Hex:EtOH=40:60 at 1 mL/min; Temp: 30° C.; Wavelength 254 nm; Rt=8.702 min) ¹H NMR (400 MHz, DMSO-d₆) δ 9.00 (s, 1H), 7.78-7.74 (m, 2H), 7.49-7.39 (m, 3.3H), 7.20-7.02 (m, 2.7H), 6.13-6.00 (m, 1H), 5.58-5.27 (m, 1H), 4.74-4.24 (m, 2H), 3.16-3.04 (m, 1H), 2.86-2.67 (m, 1H), 1.74-1.62 (m, 3H), 1.20-1.15 (m, 3H). ¹⁹F NMR (376 MHz, DMSO-d₆) δ−81.92 (s, 2F).

### Compound 8B:

LC-MS (ESI): R_{T}=4.223 min, mass calcd, for C₂₅H₂₁Cl₂F₂N₅O₃ 547.1, m/z found 548.1 [M+H]⁺. Chiral analysis (Column: Chiralpak IC 5 m 46*250 mm. Mobile Phase: Hex:EtOH=40:60 at 1 mL/min; Temp: 30° C.; Wavelength 254 nm; Rt=10.917 min). ¹H NMR (400 MHz, DMSO-d₆) δ 9.01 (s, 1H), 7.80-7.75 (m, 2H), 7.50-7.39 (m, 3.31), 7.21-7.02 (m, 2.7H), 6.11-6.00 (i, 1M), 5.58-5.29 (m, 1H), 4.67-4.26 (m, 2H), 3.18-3.12 (m, 1H), 2.84-2.67 (m, 1H), 1.69 (br s, 3H), 1.19-1.12 (m, 3H). ¹⁹F NMR (376 MHz, DMSO-d₆) δ−81.95 (s, 2F).

Compound 9A and compound 9B

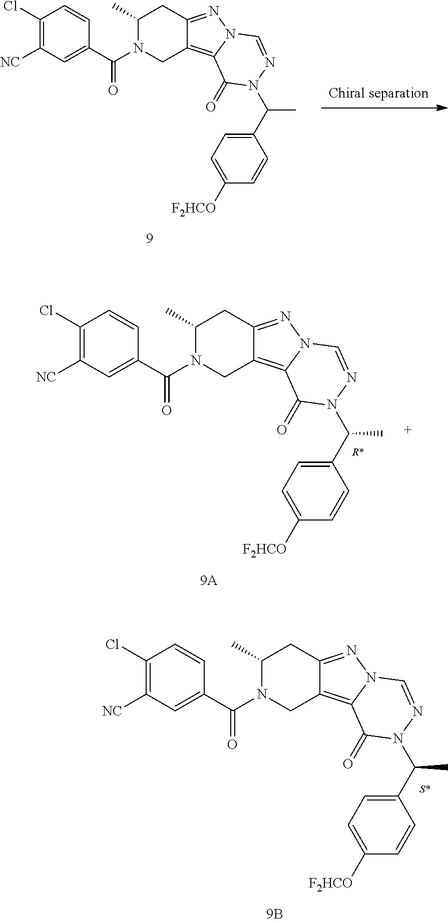 [SMILES: CC(c1ccc(OC(F)F)cc1)n1ncn2nc3c(c2c1=O)CN(C(=O)c1ccc(Cl)c(C#N)c1)[C@H](C)C3 ; "Chiral separation" ; C[C@@H]1Cc2nn3cnn([C@H](C)c4ccc(OC(F)F)cc4)c(=O)c3c2CN1C(=O)c1ccc(Cl)c(C#N)c1 ; C[C@@H]1Cc2nn3cnn([C@@H](C)c4ccc(OC(F)F)cc4)c(=O)c3c2CN1C(=O)c1ccc(Cl)c(C#N)c1]

### 2-Chloro-S—((R)-2-((R*)-1-(4-(difluoromethoxy)phenyl)ethyl)-8-methyl-1-oxo-1,2,7,8,9,10-hexahydropyrido[4′,3′:3,4]pyrazolo[1,5-d][1,2,4]triazine-9-carbonyl)benzonitrile (9A) and

### 2-chloro-5-((R)-2-((S*)-1-(4-(difluoromethoxy)phenyl)ethyl)-8-methyl-1-oxo-1,2,7,8,9,10-hexahydropyrido[4′,3′:3,4]pyrazolo[1,5-d][1,2,4]triazine-9-carbonyl)benzonitrile (9B)

2-chloro-5-((8R)-2-(1-(4-(difluoromethoxy)phenyl)ethyl)-8-methyl-1-oxo-1,2,7,8,9,10-hexahydropyrido[4′,3′3,4]pyrazolo[1,5-d][1,2,4]triazine-9-carbonyl)benzonitrile 9 (140 mg, 90% purity, 0.234 mmol) was synthesized in an analogous way to compound 7 Compound 9 was separated by chiral Prep. HPLC separation condition: (Column: Chiralpak IA 5 μm 20*250 mm; Mobile Phase: ACN:IPA=70:30 at 30 mL/min; Temp: 30° C.; Wavelength: 254 nm) to give compound 9A (18.4 mg, 49.2% purity, 14.5% yield, 99.3% stereopure) as white solids and compound 9B (22.2 mg, 99.5% purity, 17.5% yield, 99.8% stereopure) as white solids.

Compound 9A: LC-MS (ESI): R_{T}=7.569 min, mass calcd. For C₂₆H₂₁ClF₂N₆O₃ 538.9, m/z found 539.1 [M+H]⁺. Chiral analysis (Column: Chiralpak IA 5 μm 4.6*250 mm; Mobile Phase: ACN:IPA=70:30; at 1 mL/imin; Temp: 30° C.; Wavelength: 254 nm; R_{T}=4.247 min). ¹H NMR (400 MHz, CDCl₃) δ 8.35 (s, 1H), 7.75 (s, 1H), 7.60 (s, 2H), 7.47 (d. J=8.4 Hz, 2H), 7.08 (d, J=8.4 Hz, 2H), 6.48 (t, J=73.6 Hz, 1H), 6.15 (br s, 1H), 5.44 (br s, 1H), 4.91-4.58 (m, 2H), 3.17 (br s, 1H), 2.85-2.81 (m, 1H), 176 (d, J=6.8 Hz, 3), 1.26-1.25 (m, 3H). ¹⁹F NMR (376 MHz, CDCl₃) δ−80.97 (s, 2F).

Compound 9B: LC-MS (ESI): R_{T}=7.579 min. mass calcd. For (C₂₆H₂₁ClF₂N₆O₃ 538.9, m/z found 539.1 [M+H]⁺. Chiral analysis (Column: Chiralpak IA 5 μm 4.6*250 mm; Mobile Phase: ACN:IPA=70:30; at 1 mL/min, Temp: 30° C., Wavelength: 254 nm; R_{T}=6.917 min). ¹H NMR (400 MHz, CDCl₃) δ 8.35 (s, 1H), 7.75 (s, 1H), 7.60 (s, 2H), 7.48 (d, J=8.4 Hz, 2H), 7.09 (d, J=8.4 Hz, 2H), 6.48 (t, J=77.2 Hz, 1H), 6.16 (br s, 1H), 5.45 (br s, 1H), 4.93-4.61 (m, 2H), 3.17 (br s, 1H), 2.85-2.81 (m, 1H) 1.76 (d, J=72 Hz, 3), 1.26-1.25 (m, 3H). ¹⁹F NMR (376 MHz, CDCl₃) δ −80.97 (s, 2F).

### Compound 10A and Compound 10B

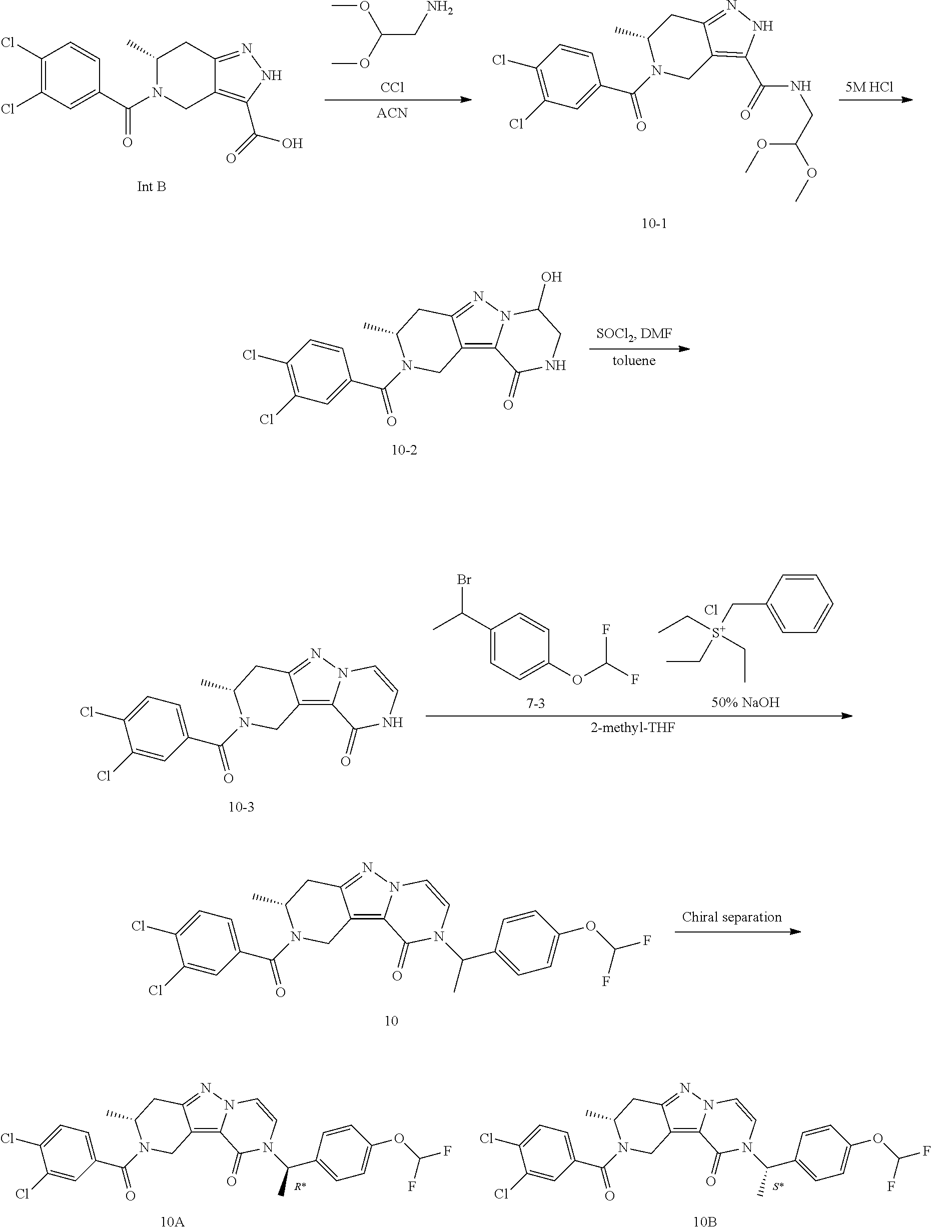

### Intermediate 10-1:

### (R)-5-(3,4-(Dichlorobenzoyl)-N-(2,2-dimethyoxyethyl)-6-methyl-4,5,6,7-tetrahydro-2H-pyrazolo[4,3-c]pyridine-3-carboxamide

To a solution of (R)-5-(3,4-(Dichlorobenzoyl)-N-(2,2-dimethyoxyethyl)-6-methyl-4,5,6,7-tetrahydro-2H-pyrazolo[4,3-c]pyridine-3-carboxylic acid Int B (1.0 g, 90% purity, 2.54 mmol) in acetonitrile (10 mL) was added di(1H-imidazol-1-yl)methanone (600 mg 3.70 mmol) at room temperature. After stirred at 60° C. for 2 hours, 2, 2-dimethoxyethanamine (350 Mg, 3.33 mmol) was added to the reaction. The reaction mixture was stirred for 3 hours at 60° C., then cooled down to room temperature and concentrated. The crude residue was diluted with water (20 mL) and extracted with ethyl acetate (20 mL) for three times. The combined organic layers were washed with brine (20 mL), dried over Na₂SO₄₍ₛ₎ and filtered. The filtrate was concentrated to give the title compound (1.0 g, 93% purity from LCMS. 83% yield) as white solids. LC-MS (ESI): R_{T}=1.52 min, mass calcd. for C₁₉H₂₂Cl₂N₄O₄ 440.1, m/z [M−H]⁻ found 439.1.

### Intermediate 10-2:

### (3R)-2-(3,4-Dichlorobenzoyl)-7-hydroxy-3-methyl-1,2,3,4,8,9-hexahydropyrido[4′,3′:3,4]pyrazolo[1,5-a]pyrazin-10(7H)-one

A solution of (R)-5-(3,4-dichlorobenzoyl)-N-(2,2-dimethoxyethyl)-6-methyl-4,5,6,7-tetrahydro-2H-pyrazolo[4,3-c]pyridine-3-carboxamide (10 g, 93% purity, 0.612 mmol) in 5 M hydrochloride aqueous solution (20 mL, 100 mmol) was stirred at 30° C. for 16 hours. The reaction mixture was diluted with water 500 mL) and extracted with ethyl acetate (30 mL) for three times. The combined organic layers were washed with brine (30 mL), dried over Na₂SO₄₍ₛ₎ and filtered. The filtrate was concentrated under reduced pressure to give a crude, which was purified by C18 column (acetonitrile:water=50% to 60%) to give the title compound (600 mg, 90% purity from ¹HNMR, 65% yield) as white solids. LC-MS (ESI): R_{T}=1.45 min, mass calcd. for C₁₇H₁₆Cl₂N₄O₃ 394.1, m/z [M+H]⁺ found 395.0. ¹H NMR (400 MHz, DMSO-d₆) δ 8.21-8.10 (m, 1H), 7.77-7.73 (m, 2H), 7.46 (t, J=6.4 Hz, 1H), 7.14-7.09 (m, 1H), 5.68 (br s, 1H), 5.50-5.20 (m, 1H), 4.59-4.37 (m, 1H), 4.24-4.11 (m, 1H), 3.83-3.69 (m, 1H), 3.43-3.35 (m, 1H), 3.02-2.89 (m, 1H), 2.67-2.57 (m, 1H), 1.13-1.06 (m, 3H)

### Intermediate 10-3:

### (R)-2-(3,4-Dichlorobenzoyl)-3-methyl-1,2,3,4-tetrahydropyrido[4′,3′:3,4]pyrazolo[1,5-a]pyrazin-10(9H)-one

To a mixture of (3R)-2-(3,4-dichlorobenzoyl)-7-hydroxy-3-methyl-1,2,3,4,8,9-hexahydropyrido[4′,3′:3,4]pyrazolo[1,5-a]pyrazin-10(7H-one 10-2 (400 mg, 95% purity, 0,880 mmol) in toluene (30 mL) was added sulfurous dichloride (230 mg, 1.93 mmol) at room temperature, followed by addition of N,N-dimethylacetamide (0.05 mL). After stirred at 50° C. for 12 hours, the reaction mixture was cooled down to room temperature, and then concentrated under reduced pressure to give a crude, which was purified by silica gel column chromatography (petroleum ether ethyl acetate=3-1 to 1-1) to give the title compound (270 mg, 90% purity from ¹HNMR, 73% yield) as brown solids, LC-MS (ESI): R_{T}=1.50 min. mass calcd. for C₁₇H₁₄Cl₂N₄O₂ 376.1, m/z found 377.1 [M+H]⁺. ¹H NMR (400 MHz, CDCl₃) δ 9.99-9.25 (m, 1H), 7.55-7.51 (m, 2H), 7.41 (d, J=6.0 Hz, 1H), 7.29-7.28 (m, 1H), 6.61 (br s, 1H), 5.07-5.74 (m, 0.4H), 5.55-5.38 (m, 0.61), 5.04-4.39 (m, 2H), 3.16 (br s, 1H), 2.80-2.76 (m, 1f), 1.31-1.21 (m, 3H).

### Compound 10:

### (3R)-2-(3,4-Dichlorobenzoyl)-9-(1-(4-(difluoromethoxy)phenyl)ethyl)-3-methyl-1,2,3,4-tetrahydropyrido[4′,3′:3,4]pyrazolo[1,5-a]pyrazin-10(9H)-one

To a solution of (R)-2-(3,4-dichlorobenzoyl)-3-methyl-1,2,3,4-tetrahydropyrido[4′,3′:3,4]pyrazolo[1,5-a]pyrazin-10(9H)-one 10-3 (270 mg, 90% purity, 0.644 mmol), 1-(1-bromoethyl)-4-(difluoromethoxy)benzene 74 (270 mg, 90% purity, 0.968 mmol) in 2-methyltetrahydrofuran (4 mL) at room temperature was added 50% sodium hydroxide (2 mL) and benzyltriethylammonium chloride (20 mg, 0.088 mmol). After stirred at 60° C. under nitrogen atmosphere for 2 hours, the reaction was quenched with water (30 mL) and extracted with ethyl acetate (50 mL) for three times. The combined organic layers were washed with brine (50 mL), dried over anhydrous Na₂SO₄₍ₛ₎ and filtered. The filtrate was evaporated under reduced pressure to afford yellow crude solids, which were purified by C18 column (acetonitrile:water=50% to 60%) to give the title compound (250 mg, 90% purity from ¹HNMR, 64% yield) as brown solids. LC-MS (ESI): R_{T}=1.77 min. mass calcd. for C₂₆H₂₂Cl₂N₄O₃ 546.1, m/z found 547.1 [M+H]⁺. ¹H NMR (400 MHz, CDCl₃) δ 7.78-7.68 (m, 3H), 7.51-7.28 (m, 3.4H), 7.22-7.17 (m, 2.6H), 7.04-691 (m, 1H), 6.21-5.96 (m, 1H), 5.65-5.53 (m, 0.5H), 5.35-5.17 (m, 0.5H), 4.73-4.60 (m, 0.7H), 4.36-4.20 (m, 1.3H), 3.12-2.99 (m, 1H), 2.78-2.58 (m, 1H), 1.76-1.64 (m, 3H), 1.16-1.10 (m, 3H).

### Compounds 10A and 108:

### (R)-2-(3,4-dichlorobenzoyl)-9-((R*)-1-(4-(difluoromethoxy)phenyl)ethyl)-3-methyl-1,2,3,4-tetrahydropyrido[4′,3′:3,4]pyrazolo[1,5-a]pyrazin-10(9H)-one (10A) and

### (R)-2-(3,4-dichlorobenzoyl)-9-((S*)-1-(4-(difluoromethoxy)phenyl)ethyl)-3-methyl-1,2,3,4-tetrahydropyrido[4′,3′:3,4]pyrazolo[1,5-a]pyrazin-10(9H)-one (10B)

A racemic mixture of (3R)-2-(3,4-dichlorobenzoyl)-9-(1-(4-(difluoromethoxy)phenylethyl)-3-methyl-1,2,3,4-tetrahydropyrido[4′,3′:3,4]pyrazolo[1,5-a]pyrazin-10(9H)-one compound 10 (20) mg, 90% purity, 0.329 mmol) was separated by chiral. HPLC (separation condition: Column: Chiralpak IC 5 μm 20*250 mm; Mobile Phase: Hex:EtOH=50:50 at 30 mL/min; Temp: 30° C.; Wavelength 254 nm) to give compound 10A (47 mg, 25.5% yield, 477% purity from LCMS, 100% stereopure) as white solids and compound 10B (41 mg, 22.4% yield, 98.5% purity from LCMS, 100% stereopure) as white solids.

### Compound 19A:

LC-MS (ESI): R_{T}=7.127 min, mass calcd. for C₂₆H₂₂Cl₂N₂O₃, 546.1, m/Z found 547.1 [M+H]⁺. Chiral analysis (Column: Chiralpak IC 5 μm 4.6*250 mm, Mobile Phase HEX:EtOH=50:50 at 1 mL/min; Temp: 30° C.; Wavelength: 254 nm, R_{T}=8.351 min) ¹H NMR (400 MHz, DMSO-d₆) δ 7.77-7.68 (m, 3H), 7.48-7.36 (m, 3.3H), 7.22-7.03 (m, 2.7H), 7.00-6.89 (m, 1H), 6.18-6.06 (m, 0.7H), 6.02-5.94 (m, 0.3H), 5.65-5.50 (m, 0.6H), 5.30-5.27 (m, 0.4H), 4.68-4.63 (m, 0.7H), 4.31-4.19 (m, 1.3H), 3.11-3.00 (m, 1H) 2.78-260 (m, 1H), 1.71 (s, 3H), 1.23-1.10 (m, 3H). ¹⁹F NMR (376 MHz, DMSO-d₆) −82.08 (s, 2F).

### Compound 10B:

LC-MS (ESI): R_{T}=7.180 min, mass calcd. for C₂₆H₂₂Cl₂N₄O₃, 546.1, m/z found 547.1 [M+H]⁺. Chiral analysis (Column: Chiralpak IC 5 μm 4.6*250 mm; Mobile Phase: HEX:EtOH=50:50 at 1 mL/min; Temp: 30° C.; Wavelength: 254 nm; R_{T}=10.356 min). ¹H NMR (400 MHz, DMSO-d₆) δ 7.78-7.74 (m, 2H), 7.70-7.68 (m, 1H), 7.48-7.41 (m, 3.2H), 7.22-7.17 (s, 2.5H), 7.04-6.96 (m, 1.3H), 6.15-5.98 (m, 1H), 5.63-5.22 (m, 0.6H), 5.34-5.22 (m, 0.4H), 4.73-4.59 (m, 0.7H), 4.39-4.14 (m, 1.3H), 3.14-2.99 (m, 1H), 2.80-2.60 (m, 1H), 1.72 (br s, 3H), 1.23-1.08 (m, 3H) ¹⁹F NMR (376 MHz, DMSO-d₆) −82.11 (s, 2F).

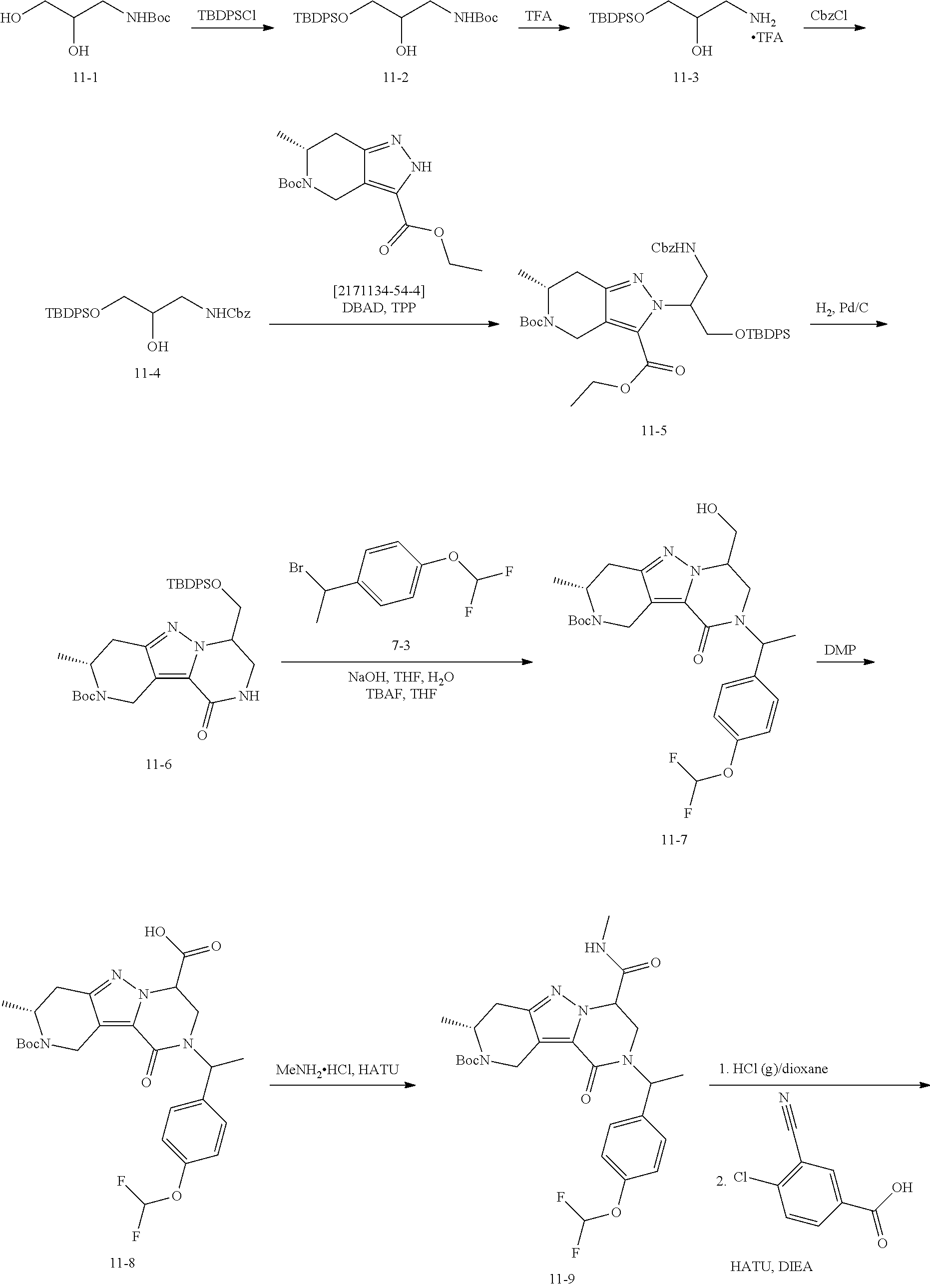

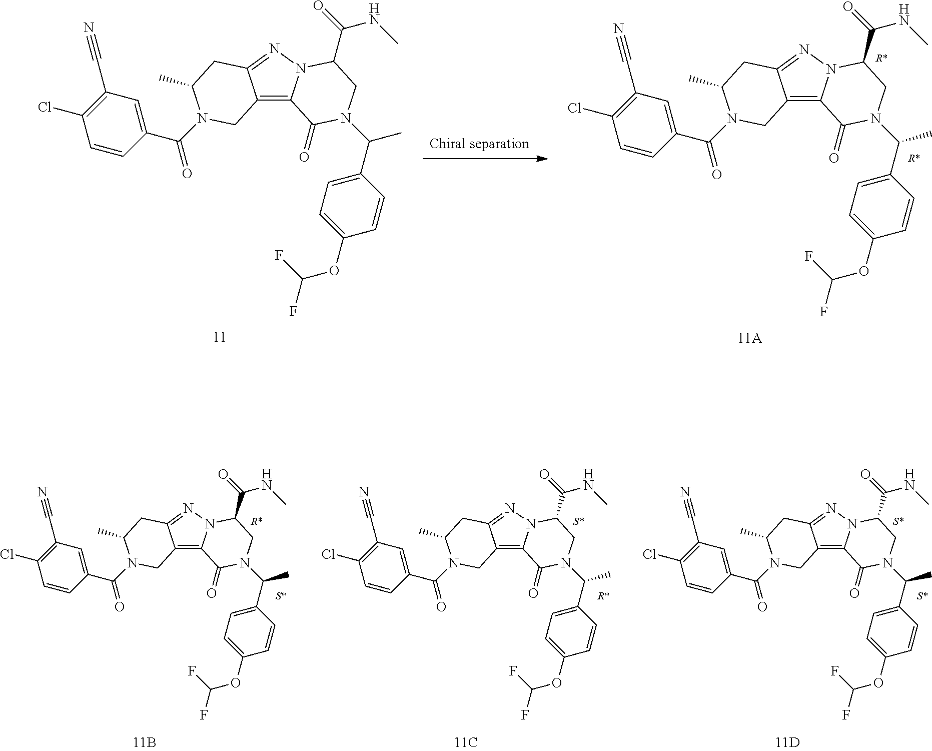 [SMILES: CNC(=O)C1CN(C(C)c2ccc(OC(F)F)cc2)C(=O)c2c3c(nn21)C[C@@H](C)N(C(=O)c1ccc(Cl)c(C#N)c1)C3 ; "Chiral separation" ; CNC(=O)[C@H]1CN([C@H](C)c2ccc(OC(F)F)cc2)C(=O)c2c3c(nn21)C[C@@H](C)N(C(=O)c1ccc(Cl)c(C#N)c1)C3 ; CNC(=O)[C@H]1CN([C@@H](C)c2ccc(OC(F)F)cc2)C(=O)c2c3c(nn21)C[C@@H](C)N(C(=O)c1ccc(Cl)c(C#N)c1)C3 ; CNC(=O)[C@@H]1CN([C@H](C)c2ccc(OC(F)F)cc2)C(=O)c2c3c(nn21)C[C@@H](C)N(C(=O)c1ccc(Cl)c(C#N)c1)C3 ; CNC(=O)[C@@H]1CN([C@@H](C)c2ccc(OC(F)F)cc2)C(=O)c2c3c(nn21)C[C@@H](C)N(C(=O)c1ccc(Cl)c(C#N)c1)C3]

### Intermediate 11-2

### tert-Butyl (3-((tert-butyldiphenylsilyl)oxy)-2-hydroxypropyl)carbonate

To a solution of tert-butyl (2,3-dihydroxypropyl)carbamate 11-1 (8 g 41.8 mmol) in dry dichloromethane (50 mL) was added triethylamine (9 g, 88.9 mmol), N,N-dimethylpyridin-4-amine (400 mg, 3.27 mmol) and tert-butylchlorodiphenylsilane (12 g, 43.7 mmol) at 0° C. The mixture was stirred at room temperature overnight. The mixture was diluted with dichloromethane (100 mL) and washed with water (150 mL) twice and brine (200 mL), dried over Na₂SO₄₍ₛ₎ and filtered. The filtrate was purified by silica gel chromatography (petroleum ether:ethyl acetate=100:1-50:1) to give the title compound (14 g, 95% purity from HNMR, 74% yield) as light yellow oil

1HNMR (400 MHz, CDCl₃) δ 7.65-7.63 (m, 4H), 7.46-7.37 (m, 6H), 4.84 (br s, 1H), 3.83-3.77 (m, 1H), 3.69-3.58 (m, 2H), 3.36-3.33 (m, 1H), 3.17-3.11 (m, 1H), 2.91-2.90 (m, 1H), 1.41 (s, 9H), 1.07 (s, 9H).

### Intermediate 11-3:

### 1-Amine-3-((tert-butyldiphenylsilyl)oxy)propan-2-ol trifluoroacetate

To a solution of tert-butyl (3-((tert-butyldiphenylsilyl)oxy)-2-hydroxypropyl)carbamate 11-2 (4.5 g, 95% purity, 9.95 mmol) in dichloromethane (50 mL) was added trifluoroacetic acid (20 mL) at 0° C. After stirred at 0° C. for 4 h, the mixture was concentrated under reduced pressure to give the title compound (4.5 g, 95% purity, 93% yield) as yellow solids R_{T}=1.55 min, mass calcd. for C₁₉H₂₂NO₂Si 329.2 m/z found 330.5 [M+H]⁺.

### Intermediate 11-4:

### Benzyl (3-((tert-butyldiphenylsilyl)oxy)-2-hydroxypropyl)carbamate

To a solution of 1-amino-3-((tert-butyldiphenylsilyl)oxy)propan-2-ol trifluoroacetate 11-3 (4.5 g, 95% purity, 9.44 mmol) and saturated sodium bicarbonate aqueous solution (20 mL) in tetrahydrofuran (20 mL) was added dropwise benzyl chloroformate (2 mL, 14.1 mmol) at 0° C. under nitrogen atmosphere. After stirred at room temperature overnight, the mixture was concentrated under reduced pressure to remove tetrahydrofuran, followed by addition of water (50 mL) and extracted with ethyl acetate (50 mL) for three times. The combined organic layers were washed with brine (100 mL), dried over Na₂SO₄₍ₛ₎ and filtered. The filtrate was concentrated and purified by silica gel column chromatography (petroleum ether:ethyl acetate=4:1) to give the title compound (4 g, 95% purity from HNMR, 87% yield) as colorless oil.

¹H NMR (400 MHz, CDCl₃) δ 7 64-7.63 (m, 4H), 7.45-7.29 (m, 11H), 5.08 (s, 2H), 3.88-3.77 (m, 1H), 3.70-3.67 (m, 1H), 3.62-3.58 (m, 1H), 3.44-3.39 (m, 1H), 3.23-3.16 (m, 1H), 2.85-266 (m, 1H), 1.06 (s, 9H).

### Intermediate 11-5:

### (6R)-5-tert-Butyl 3-ethyl 2-(10,10-dimethyl-3-oxo-1,9,9-triphenyl-2,8-dioxa-4-aza-9-silaundecan-6-yl)-6-methyl-6,7-dihydro-2H-pyrazolo[4,3-c]pyridine-3,5(4H)-dicarboxylate

To a tetrahydrofuran solution (40 mL) of benzyl (3-((tert-butyldiphenylsilyl)oxy)-2-hydroxypropyl)carbamate 11-4 (2.5 g, 95% purity, 5.12 mmol) and (R)-5-tert-butyl 3-ethyl 6-methyl-6,7-dihydro-2H-pyrazolo[4,3-c]pyridine-3,5(4H)-dicarboxylate (1.8 g, 5.53 mmol, purity 95%) was added triphenylphosphine (2.7 g, 10.3 mmol) aid di-tert-butyl diazene-1,2-dicarboxylate (2.3 g, 9.99 mmol) at 0° C. After stirred at 50° C. for 4 hours, the mixture was concentrated under reduced pressure to give a residue. The crude residue was purified by C18 column (acetonitrile:water=5% to 100%) to give the title compound (3 g, 95% purity from HNMR, 74% yield) as white solids.

¹HNMR (400 MHz, CDCl₃) δ 7.66-7.32 (m, 15H), 5.73-5.65 (m, 0.8H), 5.30-4.57 (m, 5.2H), 4.35-3.72 (m, 6H), 3.17-2.82 (m, 1H), 2.67-2.45 (m, 1H), 1.51-1.33 (m, 12H), 1.11-0.92 (m, 12H).

### Intermediate 11-6:

### (3R)-tert-Butyl 7-(((tert-butyldiphenylsilyl)oxy)methyl)-3-methyl-10-oxo-3,4,7,8,9,10-hexahydropyrido[4′,3′:3,4]pyrazolo[1,5-a]pyrazine-2(1H)-carboxylate

To a methanol solution (20 mL) of (6R)-5-tert-butyl 3-ethyl 2-(10,10-dimethyl-3-oxo-1,9,9-triphenyl-2,8-dioxa-4-aza-9-silaundecan-6-yl)-6-methyl-6,7-dihydro-2H-pyrazolo[4,3-c]pyridine-3,5(4H)-dicarboxylate 11-5 (1 g, 95% purity, 1.26 mmol) was added 10% palladium on activated carbon (100 mg) at room temperature. After stirred at 45° C. under 15 psi hydrogen atmosphere overnight, the mixture was filtered and concentrated to give the title compound 700 mg, 95% purity from HNMR, 92% yield) as light yellow oil.

¹HNMR (400 MHz, CDCl₃) δ 7.69-7.30 (m, 10H), 5.72-5.51 (m, 1H), 5.10-4.77 (m, 2H), 4.54-3.76 (m, 6H), 2.45-2.86 (m, 1H), 2.63-2.52 (m, 1H), 1.49-1.46 (m, 9H), 1.09-0.81 (m, 12H).

### Intermediate 11-7

### (3R)-tert-Buty 9-(1-(4-(difluoromethoxy)phenyl)ethyl)-7-(hydroxymethyl)-3-methyl-10-oxo-3,4,7,8,9,10-hexahydropyrido[4′,3′:3,4]pyrazolo[1,5-a]pyrazine-2(1H)-carboxylate

To a solution of (3R)-tert-butyl 7-(((tert-butyldiphenylsilyl)oxy)methyl)-3-methyl-10-oxo-3,4,7,8,9,10-hexahydropyrido[4′,3′:3,4]pyrazolo[1,5-a]pyrazine-2(11H)-carboxylate 11-6 (1.7 g, 2.81 mmol, 95% purity) and 1-(1-bromoethyl-4-(difluoromethoxy)benzene 7-3 (1.2 g, 4.78 mmol) in 2-methyltetrahydrofuran (8 mL) was added 50% sodium hydroxide in water (8 mL) slowly at 30° C. After stirred at 30° C. for 2 hours, the mixture was diluted with water (50 mL) and concentrated at room temperature under reduced pressure to remove the volatile. The remained aqueous layer was acidified with 2M hydrochloride aqueous solution (50 mL) and extracted with ethyl acetate (50 mL) twice. The combined organic layers were washed with brine (100 mL), dried over Na₂SO₄₍ₛ₎ and filtered. The filtrate was concentrated to give the crude. The crude product was then dissolved in tetrahydrofuran (15 mL), followed by addition of 1 M tetrabutylammonium fluoride in tetrahydrofuran (5 mL, 5 mmol) at 0° C. After stirred at 0° C. for 4 hour; the mixture was filtered and concentrated under reduced pressure to give crude. The crude was purified by C18 column (acetonitrile:water=5% to 100%) to give the title compound (1.2 g, 95% purity from HNMR, 80% yield) as yellow oil. R_{T}=1.65 min, mass calcd. for C₂₅H₃₂F₂N₄O₅ 506.2 m/z found 507.1 [M+H]⁺.

### Intermediate 11-8:

### (3R)-2-(tert-Butoxycarbonyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-7-carboxylic acid

To the solution of (3R)-tert-butyl 9-(1-(4-(difluoromethoxy)phenyl)ethyl)-7-(hydroxymethyl)-3-methyl-10-oxo-3,4,7,8,9,10-hexahydropyrido[4′,3′:3,4]pyrazolo[1,5-a]pyrazine-2(1H)-carboxylate 11-7 (1.2 g, 2.25 mmol, 95% purity) in dichloromethane (20 mL) was added Dess-Martin periodinane (2 g, 4.72 mmol) under nitrogen atmosphere at 0° C. After stirred at 30° C. for 2 hours, the mixture was adjusted to pH˜8 with sodium bicarbonate aqueous solution, and extracted with dichloromethane (40 mL) twice. The combined organic layers were washed with brine (80 mL) and dried over Na₂SO₄₍ₛ₎ and filtered. The filtrate was concentrated and purified by CIS column (acetonitrile:water 5% to 100%) to give the title compound (500 ng, 93% purity, 40% yield) as white solids. R_{T}=1.35 min, mass calcd. for C₂₅H₃₀F₂N₄O₆ 520.2 m/z found 521.1 [M+H]⁺.

### Intermediate 11-9:

### (3R)-tert-Butyl 9-(1-(4-(difluoromethoxy)phenyl)ethyl)-3-methyl-7-(methylcarbamoyl)-10-oxo-3,4,7,8,9,10-hexahydropyrido[4′,3′:3,4]pyrazolo[1,5-a]pyrazine-2(1H)-carboxylate

To a N,N-dimethylformamide solution (5 mL) of (3R)-2-(tert-butoxycarbonyl)-9-(1-(4-(difluoromethoxy)phenyl ethyl)-3-methyl-10-oxo-1,2,3,4,7,8,9,10-octahydropyrido[4′,3′:3,4]pyrazolo[1,5-a]pyrazine-7-carboxylic acid 11-8 (500 mg, 93% purity, 0.893 mmol) was added methanamine hydrochloride (130 mg, 1.93 mmol), N-ethyl-N-isopropylpropan-2-amine (450 mg, 3.48 mmol) and O-(7-Azabenzotriazol-1-yl)-N,N,N′,N′-tetramethyluronium hexafluorophosphate (850) mg, 2.24 mmol) at 0° C. After stirred at 30° C. for 16 hours, the mixture was concentrated under reduced pressure to give a residue. The crude residue was purified by C18 column (acetonitrile:water=5% to 100%) to give the title compound 400 mg, 90% purity from HNMR, 76% yield) as white solids.

¹HNMR (400 MHz, CDCl₃) δ 7.41-737 (m, 1.31), 7.33-7.28 (m, 0.7H), 7.12-7.08 (m, 2H), 6.70-6.32 (m, 1H), 6.05-5.88 (m, 2H), 5.27-5.16 (m, 1H), 4.998-4.81 (m, 2H), 4.31-4.21 (m, 1H), 4.08-4.04 (m, 0.6H), 3.91-3.80 (m, 0.7H), 3.44-3.35 (m, 0.7H), 3.04-2.93 (m, 1H), 2.79-2.77 (m, 2H), 2.67-2.59 (m, 0.2H), 1.59-1.56 (m, 3H), 1.50-1.46 (m, 9H), 1.18-1.11 (m, 3).

### Compound 11:

### (3R)-2-(4-Chloro-3-cyanobenzoyl)-9-(1-(4-difluoromethoxy)phenyl)ethyl)-N,3-dimethyl-10-oxo-1,2,3,4,7,8,9,10-octahydropyrido[4′3′:3,4]pyrazolo[1,5-a]pyrazine-7-carboxamide

A solution of (3R)-tert-butyl 9-(1-(4-(difluoromethoxy)phenyl)ethyl)-3-methyl-7-(methylcarbamoyl)-10-oxo-3,4,7,8,9,10-hexahydropyrido[4′,3′:3,4]pyrazolo[1,5-a]pyrazine-2(1H)-carboxylate 11-9 (400 mg, 0.075 mmol, 90% purity) in 4 M hydrochloride in dioxane (5 mL 20 mmol) was stirred at 25° C. for 3 hours. The reaction mixture was then concentrated to give a residue. To a N,N-dimethylformamide solution (4 mL) of the crude residue was added 4-chloro-3-cyanobenzoic acid (200 mg, 1.10 mmol), N-ethyl-N-isopropylpropan-2-amine (500 mg, 3.87 mmol) and O-(7-azabenzotriazol-1-yl)-N,N,N′,N′-tetramethyluronium hexafluorophosphate (600 mg, 1.58 mmol) at 0° C. After stirred at 30° C. for 2 hours, the mixture was concentrated under reduced pressure to give a crude residue, which was purified by C18 column (acetonitrile:water=5% to 100%) to give the title compound (320 mg, 75% yield, 95% purity) as white solids. R_{T}=1.61 min, mass calcd. for C₂₅H₂₇ClF₂N₆O₄ 596.2 m/z found 596.9 [M+H]⁺.

### Compounds 11A & 11B & 11C & 11D:

### (3R,7R*)-2-(4-chloro-3-cyanobenzoyl)-9-((R*)-1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide (11A) and

### (3R,7R*)-2-(4-chloro-3-cyanobenzoyl-9-((S*)-1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide (11B) and

### (3R,7S*)-2-(4-chloro-3-cyanobenzoyl)-9(R*)-1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide (11C) and

### (3R,7S*)-2-(4-chloro-3-cyanobenzoyl)-9-((S*)-1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide (11D)

A racemate of (3R)-2-(4-chloro-3-cyanobenzoyl)-9-(1-(4-(difluoromethoxy)phenyl)ethyl)-N,3-dimethyl-10-oxo-1,2,3,4,7,8,9,10-octahydropyrido[4′,3′:3,4]pyrazolo[1,5-a]pyrazine-7-carboxamide compound 11 (400 mg, 95% purity, 0.637 mmol) was separated by chiral HPLC (separation condition. Column: Chiralpak IA, 5 μm 30*250 mm, MeOH:DCM=80:20, 25 ml/min, Col Temp: 30° C.; Wavelength: 254 nm) to afford the desired Fraction A (100 mg, mixture of compound 11A & compound 11B), compound 11C (77 mg, 99.9% purity, 20.3% yield, 99.8% stereopure) and compound 11D (40 mg, 99.8% purity, 10.5% yield, 99.6% stereopure) as white solids. Fraction A (100 mg) was separated by chiral HPLC (separation condition: Column: Chiralpak IE, 5 μm 30*250 mm, Hex:EtOH=40:60, 20 ml/min; Col. Temp: 30° C.; Wavelength: 254 nm) to afford the desired product compound h A (25 mg, 96.8% purity, 6.37% yield, 100% stereopure) and compound 11B (25 mg, 99.0% purity, 6.51% yield, 100% stereopure) as white solids.

### Compound 11A:

R_{T}=2.206 min, mass calcd. for C₂₉H₂₇ClF₂N₆O₄ 596.2 m/z found 597.2 [M+H]⁺.

Chiral HPLC (Column: Chiralpak IE 5 μm 4.6*250 mm, Mobile Phase: Hexane:EtOH=40:60 at 1.0 mL/min; Temp: 30° C.; Wavelength: 254 nm, R_{T}=10.639 min)

¹HNMR (400 MHz, CDCl₃) 7.74 (s, 1H), 7.60 (s, 2H), 7.30 (d, J=7.6 Hz, 2H), 7.09 (d, J=8.0 Hz, 2H), 6.51 (t, J=73.6 Hz, 1H), 6.10-−5.40 (m, 3H), 4.85-4.26 (m, 3H), 3.91-3.81 (m, 2H), 3.17-2.97 (m, 1H), 2.76-2.68 (m, 4H), 1.68-1.57 (m, 1.32 (d, J=6.0H, 3H). ¹F NMR (376 MHz, CDCl₃) δ−80.89 (s, 2F).

### Compound 1B:

R_{T}=3.584 min, mass calcd. for C₂₉H₂₇ClF₂N₆O₄ 506.2 m/z found 597.1 [M+H]⁺.

Chiral HPLC (Column: Chiralpak IE 5 μm 4.6*250 mm. Mobile Phase: Hexane:EtOH=40:60 at 1.0 mL/min, Temp, 30° C., Wavelength: 254 nm. R_{T}=13.735 min).

¹HNMR (400 MHz, CDCl₃) δ 7.74 (s, 1H), 7.61 (s, 2H), 7.35 (d, J=7.6 Hz, 2H), 7.11 (d, J=8.4 Hz, 2H), 6.50 (t, J=73.6 Hz, 1H), 5.98-5.36 (m, 3H), 4.83-4.51 (m, 3H), 4.09-4.06 (m, 1H), 3.39 (dd, J=13.6, 5.2 Hz, 1H), 3.13-2.99 (m, 1H), 2.81-2.72 (m, 4H), 1.58-1.57 (m, 3H), 1.32 (d, J=6.8 Hz, 3H). ¹F NMR (376 MHz, CDCl₃) δ −81.11 (s, 2F).

### Compound 11C:

R_{T}=2.604 min, mass calcd. for C₂₉H₂₇ClF₂N₆O₄ 596.2 m/z found 597.2 [M+H]⁺.

Chiral HPLC (Column: Chiralpak IA 5 μm 4.6*250 mm; Mobile Phase: MeOH:DCM=80:20 at 1.0 mL/min; Temp 30° C., Wavelength: 254 nm. R_{T}=5.941 min). ¹HNMR 400 MHz, CDCl₃) δ 7.78 (s, 1H), 7.65-7.59 (m, 2H), 7.38 (d, J=8.0 Hz, 2H), 7.11 (d, J=8.8 Hz, 2H), 6.50 (t, J=74.0 Hz, 1H), 5.96-5.43 (m, 3H), 4.83-4.30 (m, 3H), 4.09 (dd, J=13.6, 3.2 Hz, 1H), 3.43 (dd, J=13.6, 4.8 Hz, 1H), 3.21-3.04 (m, 1H), 2.70 (d, J=4.8 Hz, 3H), 3.71 (d, J=17.6 Hz, 1H), 1.56-0.1.55 (m, 3H), 1.27 (d, J=6.0 Hz, 3H). ¹F NMR (376 MHz, CDCl₃) δ−81.09 (s, 2F).

### Compound 11D.

R_{T}=2.405 min, mass calcd. for C₂₉H₂₇ClF₂N₆O₄ 596.2 m/z found 597.2 [M+H]⁺.

Chiral HPLC (Column: Chiralpak IA 5 μm 4.6*250 mm; Mobile Phase: MeOH DCM=80:20 at 10 mL/min; Temp: 30° C.; Wavelength: 254 nm, R_{T}=13.281 min). ¹HNMR (400 MHz, CDCl₃) δ 7.77 (s, 1H), 7.65-7.60 (m, 2H), 7.29-7.26 (m, 2H), 7.08 (d, J=8.4 Hz, 2H), 6.50 (t, J=73.6 Hz, 1H), 5.95-5.33 (m, 3H), 4.86-4.30 (m, 3H), 3.92-3.90 (m, 2H), 3.21-2.97 (m, 1H), 2.73-2.68 (m, 411), 1.60 (d, J=7.21 Hz, 3H), 1.27 (d, J=6.4 Hz, 3H). ¹F NMR 376 MHz, CDCl₃) δ−80.89 (s, 2F).

### Compound 12, 13B, and 13D

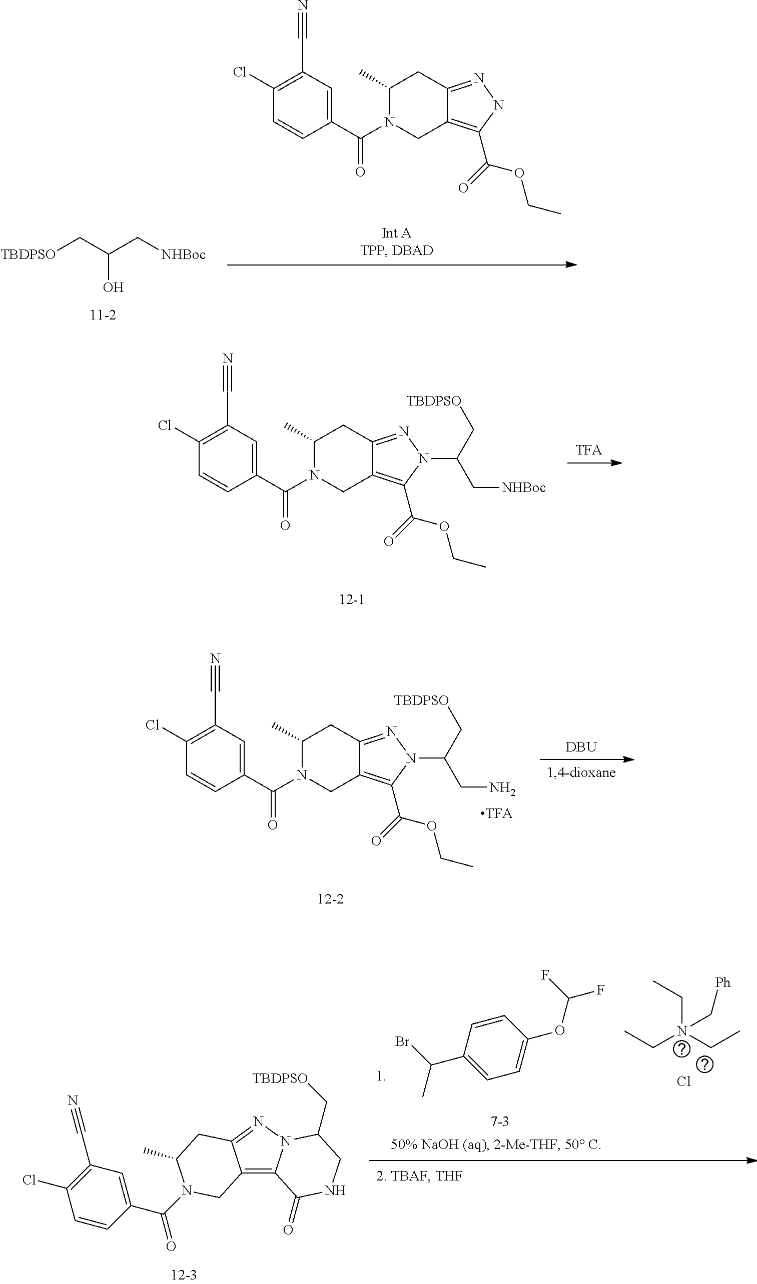

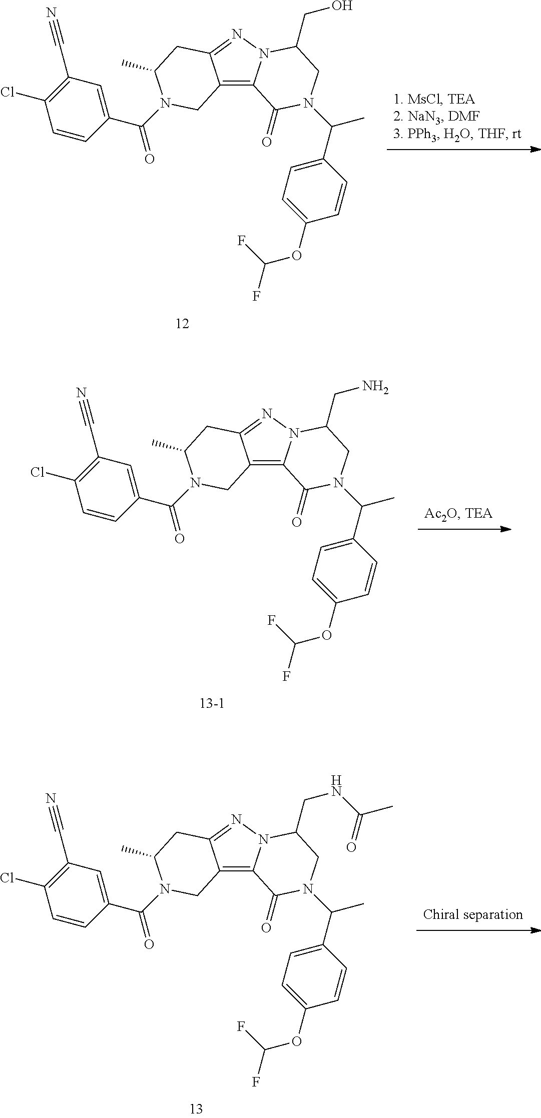 [SMILES: CC(c1ccc(OC(F)F)cc1)N1CC(CO)n2nc3c(c2C1=O)CN(C(=O)c1ccc(Cl)c(C#N)c1)[C@H](C)C3 ; CC(c1ccc(OC(F)F)cc1)N1CC(CN)n2nc3c(c2C1=O)CN(C(=O)c1ccc(Cl)c(C#N)c1)[C@H](C)C3 ; CC(=O)NCC1CN(C(C)c2ccc(OC(F)F)cc2)C(=O)c2c3c(nn21)C[C@@H](C)N(C(=O)c1ccc(Cl)c(C#N)c1)C3]

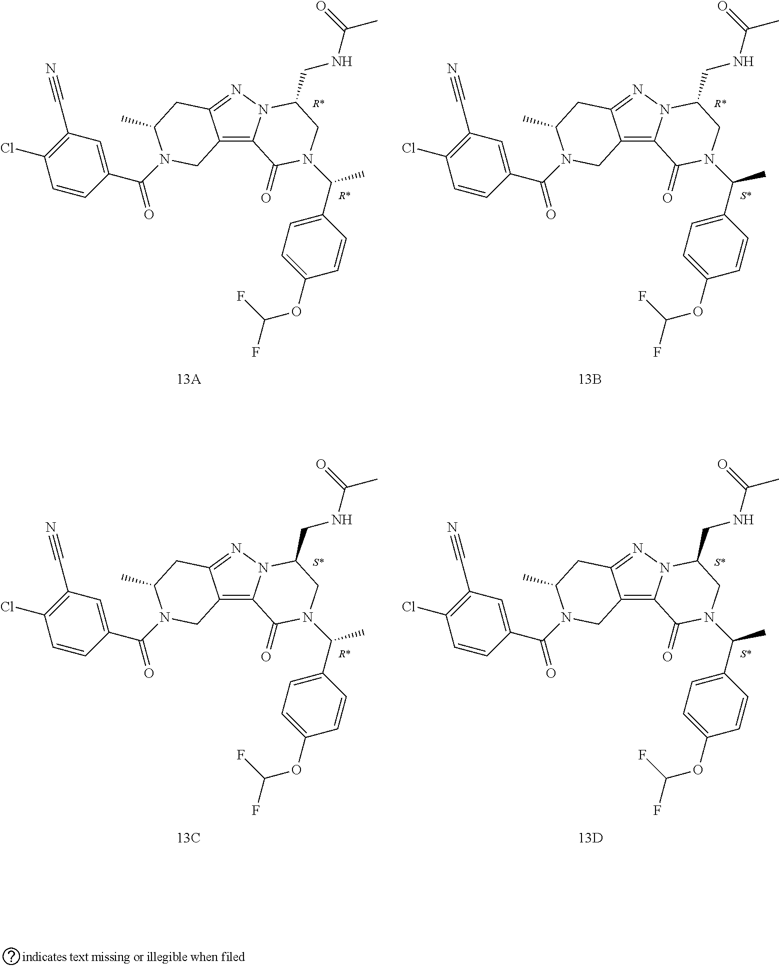 [SMILES: CC(=O)NC[C@@H]1CN([C@H](C)c2ccc(OC(F)F)cc2)C(=O)c2c3c(nn21)C[C@@H](C)N(C(=O)c1ccc(Cl)c(C#N)c1)C3 ; CC(=O)NC[C@@H]1CN([C@@H](C)c2ccc(OC(F)F)cc2)C(=O)c2c3c(nn21)C[C@@H](C)N(C(=O)c1ccc(Cl)c(C#N)c1)C3 ; CC(=O)NC[C@H]1CN([C@H](C)c2ccc(OC(F)F)cc2)C(=O)c2c3c(nn21)C[C@@H](C)N(C(=O)c1ccc(Cl)c(C#N)c1)C3 ; CC(=O)NC[C@H]1CN([C@@H](C)c2ccc(OC(F)F)cc2)C(=O)c2c3c(nn21)C[C@@H](C)N(C(=O)c1ccc(Cl)c(C#N)c1)C3]

### Intermediate 12-1:

### (6R)-Ethyl 5-(4-chloro-3-cyanobenzoyl)-6-methyl-2-(2,2,11,11-tetramethyl-9-oxo-3,3-diphenyl-4,10-dioxa-8-aza-3-siladodecan-6-yl)-4,5,6,7-tetrahydro-2H-pyrazolo[4,3-c]pyridine-3-carboxylate

To a solution of (R)-ethyl 5-(4-chloro-3-cyanobenzoyl)-6-methyl-4,5,6,7-tetrahydro-2H-pyrazolo[4,3-c]pyridine-3-carboxylate Int A (1.5 g, 90% purity, 3.62 mmol), tert-butyl (3-((tert-butyldiphenylsilyl)oxy)-2-hydroxypropyl)carbamate 11-2 (2 g, 95% purity, 4.42 mmol) and triphenylphosphine (1.35 g, 5.15 mmol) in tetrahydrofuran (20 mL) was added di-tert-butyl azodicarboxylate (1.2 g, 5.21 mmol). After stirred at 50° C. overnight, the reaction was cooled down to room temperature. The reaction mixture was poured into water (30 mL) and extracted with ethyl acetate (30 mL) twice. The combined organic layers were washed with brine (30 mL), dried over Na₂SO₄₍ₛ₎, and filtered. The filtrate was concentrated and purified by silica gel column chromatography (petroleum ether:ethyl acetate=3:1) to give the title compound (2.2 g, 07% purity from LCMS, 75% yield) as white solids. LC-MS (ESI): R_{T}=2.18, 2.23 min, mass calcd. for C₄₂H₅₀ClN₅O₆Si 783.3, m/z found 784.0 [M++H]⁺.

### Intermediate 12-2:

### (6R)-Ethyl 2-(1-amino-3-((tert-butyldiphenylsilyl)oxy)propan-2-yl)-5-(4-chloro-3-cyanobenzoyl)-6-methyl-4,5,6,7-tetrahydro-2H-pyrazolo[4,3-c]pyridine-3-carboxylate

To a solution of (6R)-ethyl 5-(4-chloro-3-cyanobenzoyl)-6-methyl-2-(2,2,11,11-tetramethyl-9-oxo-3,3-diphenyl-4,10-dioxa-8-aza-3-siladodecan-6-yl)-4,5,6,7-tetrahydro-2H-pyrazolo[4,3-c]pyridine-3-carboxylate 12-1 (2.2 g, 97% purity, 2.84 mmol) in dichloromethane (10 mL) was added trifluoroacetic acid (5 mL). After stirred at 0° C. for 1 hour, the mixture was concentrated under reduced pressure to give the title compound (2.3 g, 94% purity from LCMS, 95% yield) as a colorless oil LC-MS (ESI): R_{T}=1.26 min and 1.39 min, mass calcd. for C₃₇H₄₂ClN₅O₄Si 683.3, m/z found 684.1 [M+H]⁺.

### Intermediate 12-3:

### 5-((3R)-7-(((tert-Butyldiphenylsilyl)oxy)methyl)-3-methyl-10-oxo-1,2,3,4,7,8,9,10-octahydropyrido[4′,3′:3,4]pyrazolo[1,5-a]pyrazine-2-carbonyl)-2-chlorobenzonitrile

To a solution of (6R)-ethyl 2-(1-amino-3-((tert-butyldiphenylsilyl)oxy)propan-2-yl)-5-(4-chloro-3-cyanobenzoyl)-6-methyl-4,5,6,7-tetrahydro-2H-pyrazolo[4,3-c]pyridine-3-carboxylate trifluoroacetate 12-2 (2.3 g, 94% purity, 2.71 mmol) in 1,4-dioxane (100 mL) was added 1,5,7-triazabicyclo[4.4.0]dec-5-ene (1.9 g 13.7 mmol). After stirred at 100° C. for 2 hours, the reaction mixture was cooled down, concentrated, poured into water (30 mL), and extracted with ethyl acetate (20 ml) three times. The combined organic layers were washed with brine (20 mL), dried over Na₂SO₄₍ₛ₎, and filtered. The filtrate was concentrated and purified by C18 column (acetonitrile:water=90% to 100%) to give the title compound (1.7 g, 86% purity from LCMS, 85% yield) as white solids, LC-MS (ESI): R_{T}=1.35 min and 1.40 min, mass calcd. for C₃₅H₃₆ClN₅O₃Si 637.2, m/z found 638.4 [M+H]⁺. ¹H NMR (400 MHz, CDCl₃) δ 7.75-755 m, 7H), 7.47-7.34 (m, 6H), 6.13-5.32 (m, 2H) 4.80-4.19 (m, 3H), 4.08-4.02 (m, 1H), 3.94-3.78 (m, 2H), 3.21-2.87 (m, 1H), 2.66-2.62 (m, 1H), 1.32-1.16 (m, 3), 1.07-1.05 (m, 9H).

### Compound 12:

### 2-Chloro-5-((3R)-9-(1-(4-(difluoromethoxy)phenyl)ethyl)-7-(hydroxymethyl)-3-methyl-10-oxo-1,2,3,4,7,8,9,10-octahydropyrido[4′,3′:3,4]pyrazolo[1,5-a]pyrazine-2-carbonyl)benzonitrile

To a solution of 5-((3R)-7-(((tert-butyldiphenylsilyl)oxy)methyl)-3-methyl-10-oxo-1,2,3,4,7,8,9,10-octahydropyrido[4′,3′:3,4]pyrazolo[1,5-a]pyrazine-2-carbonyl)-2-chlorobenzonitrile 12-3 (200 mg, 86% purity, 0.269 mmol) and 1-(1-bromoethyl)-4-(difluoromethoxy)benzene 7-3 (100 mg, 0° % purity, 0358 mmol) in 2-methyltetrahydrofuran (4 mL) was added 50% sodium hydroxide aqueous solution (2 mL) and benzyltriethylammonium chloride (6 mg, 0.026 mmol). After stirred at 50° C. for 2 hours, the reaction mixture was cooled down to room temperature, diluted with water (10 mL), concentrated to remove 2-methyltetrahydrofuran, and extracted with ethyl acetate (10 mL) for three times. The combined organic layers were washed with brine (10 mL, dried over Na₂SO₄₍ₛ₎, filtered. The filtrate was concentrated to give a yellow residue. To the above yellow residue in tetrahydrofuran (0.6 mL) was added 1 M tetrabutylammonium fluoride in tetrahydrofuran (0.6 mL, 0.6 mmol). After stirred at room temperature for 1 hour, the reaction mixture was diluted with water (5 ml, and extracted with ethyl acetate (10 mL) twice. The combined organic layers were washed with brine (10 mL), dried over Na₂SO₄₍ₛ₎, and filtered. The filtrate was concentrated and purified by C18 column (acetonitrile:water=60% to 75%) to give the title compound (120 mg, 98.0% purity from LCMS, 66% yield) as white solids. LC-MS (ESI): R_{T}=2.868 min. 3.055 min. 3.202 min, 3.315 min, mass calcd. for C₂₈H₂₆ClN₂N₅O₄ 569.2, m/z found 570.2 [M+H]⁻. ¹H NMR (400 MHz, CDCl₃) δ 7.74 (s, 1H), 7.60 (s, 2), 7.43-7.29 (m, 2H), 7.17-7.08 (m, 2), 6.73-6.33 (m, 1H), 6.17-5.25 (m, 2H), 4.90-4.22 (m, 3H), 4.04-3.88 (m, 1H), 3.83-3.23 (m, 2H), 3.19-2.91 (m, 2H), 2.75-2.60 (m, 1H), 1.61-1.53 (m, 3H), 1.35-1.22 (m, 3H). ¹⁹F NMR (376 MHz. CDCl₃) δ−81.11-81.24 (m, 2F)

### Intermediate 13-1:

### 5-((3R)-7-(Aminomethyl)-9-(1-(4-(difluoromethoxy)phenyl)ethyl)-3-methyl-10-oxo-1,2,3,4,7,8,9,10-octahydropyrido[4′3′:3,4]pyrrolo[1,5-a]pyrazine-2-carbonyl)-2-chlorobenzonitrile

To a solution of 2-chloro-5-((3R)-9-(1-(4-(difluoromethoxy)phenyl)ethyl)-7-(hydroxymethyl)-3-methyl-10-oxo-1,2,3,4,7,8,9,10-octahydropyrido[4′3′:3,4]pyrazolo[1,5-a]pyrazine-2-carbonyl)benzonitrile compound 12 (1.1 g, 93% purity, 1.80 mmol) in dichloromethane (12 mL) was added mesyl chloride (0.2 mL 2.62 mmol) and triethylamine (0.8 mL, 5.76 mmol) at 0° C. After stirred at room temperature for 30 minutes, the reaction mixture was quenched with water (10 mL), extracted with ethyl acetate (15 mL) twice. The combined organic layers were dried over Na₂SO₄₍ₛ₎ and filtered. The filtrate was concentrated to give a residue. To the solution of the above residue in N,N-dimethylformamide (14 mL) was added sodium azide (800 ng, 12.3 mmol). After stirred at 50° C. for 24 hours, the reaction mixture was cooled down to room temperature, diluted with water (14 mL) and tetrahydrofuran (14 ml), followed by addition of triphenylphosphine (6.4 g, 2.44 mmol). After stirred for 1 hour, the reaction mixture was extracted with ethyl acetate (15 mL) for three times. The combined organic layers were dried over Na₂SO₄₍ₛ₎ and filtered. The filtrate was concentrated and purified by silica gel column chromatography (petroleum ether ethyl acetate=1:1 to dichloromethane:methanol=10:1) to give the title compound (1.1 g, 33% purity from LCMS, 36% yield) as yellow solids. LC-MS (ESI): R_{T}=1.65 min, mass calcd. for C₂₈H₂₇ClN₂N₆O₃ 568.2, m/z found 568.9 [M+H]⁺.

### Compound 13:

### N-(((3R)-2-(4-Chloro-3-cyanobenzoyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-]pyrazin-7-yl)methyl)acetamide

To a solution of 5-((3R)-7-(aminomethyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazine-2-carbonyl)-2-chlorobenzonitrile 13-1 (1.1 g, 33% purity, 0,638 mmol) in dichloromethane (8 mL) was added acetic anhydride (100 mg, 0.882 mmol) and triethylamine (0.2 mL, 1.44 mmol) at 0° C. After stirred at the temperature for 1 hour, the reaction mixture was poured into water (5 mL) and extracted with ethyl acetate (10 mL) for three times. The combined organic layers were washed with brine (5 mL), dried over Na₂SO₄₍ₛ₎ and filtered. The filtrate was concentrated and purified by silica gel column chromatography (petroleum ether:ethyl acetate=1:1 to dichloromethane:methanol=10:1) to give a crude, which was purified by C18 column (acetonitrile:water=50% to 65%) to give the title compound (220 mg, 97% purity from LCMS, 55% yield) as white solids, LC-MS (ESI): R_{T}=1.54 min, mass calcd. for C₃₆H₂₉ClF₂N₆O₄S 610.2, m/z found 610.9 [M+H]⁺.

### Compounds 13A & 13B & 13C & 13D:

### N-(((3R,7R*)-2-(4-Chloro-3-cyanobenzoyl)-9-((R*)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazin-7-yl)methyl)acetamide (13A) &

### N-(((3R,7R*)-2-(4-chloro-3-cyanobenzoyl)-9(S*)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazin-7-yl)methyl)acetamide (13B) &

### N-(((3R,7S*)-2-(4-chloro-3-cyanobenzoyl)-9-((R*)-1-(4-(difluoromethoxy)phenyl)ethyl)-3-methyl-10-oxo-1,2,3,4,7,8,9,10-octahydropyrido[4′3′:3,4]pyrazol[1,5-a]pyrazin-7-yl)methyl)acetamide (13C) &

### N-(((3R,7S*)-2-(4-chloro-3-cyanobenzoyl)-9-((S*)-1-(4-(difluoromethoxy)phenyl)ethyl)-3-methyl-10-oxo-1,2,3,4,7,8,9,10-octahydropyrido[4′,3′:3,4]pyrazolo[1,5-a]pyrazin-7-yl)methyl)acetamide (13D)

A racemic of N-(((3R)-2-(4-chloro-3-cyanobenzoyl)-9-(1-(4-(difluoromethoxy)phenyl)ethyl)-3-methyl-10-oxo-1,2,3,4,7,8,9,10-octahydropyrido[4′,3′:3,4]pyrazolo[1,5-a]pyrazin-7-yl)methyl)acetamide compound 13 (220 mg 97% purity, 0.349 mmol) was separated by chiral Prep. HPLC (separation condition. Column: Chiralpak IA 5 μm 30*250 mm; Mobile Phase CO₂:EtOH=60:40, at 25 g/min; Col. Temp: 40° C.; Wavelength: 214 nm) to afford Fraction I and the two crude pure chiral isomers which were further purified with CIS column (acetonitrile:water(+0.2% ammonium bicarbonate)=5% to 95%) to give compound 13A (15 mg, 98.6% purity from LCMS, 7% yield, 100% stereopure) and compound 13D (12 mg, 98.8% purity from LCMS, 6% yield, 97.8% stereopure) as white solids. Fraction I was separated by chiral Prep. HPLC (separation condition: Column: Chiralpak IB N-5.5 μm 30*250 mm; Mobile Phase: CO₂:MeOH=75:25, at 25 g/min; Col Temp 40° C.; Wavelength: 214 nm) to afford the another two crude pure chiral isomers, which was purified by C18 column (acetonitrile:water(+0.2% ammonium bicarbonate)=5% to 95%) to give compound 13B (26 mg, 99.9% purity from LCMS, 12% yield, 100% stereopure) and compound 13C (31 mg, 99.6% purity front LCMS, 14% Yield, 100% stereopure) as white solids.

### Compound 13A:

LC-MS (ESI) R_{T}=4.023 min, mass calcd. for C₃₀H₂₉ClF₂N₆O₄S 610.2, m/z found 611.3 [M+H]⁺. Chiral HPLC (Column: Chiralpak IA 5 μm 4.6*250 mm, Mobile Phase: CO₂:EtOH=60:40 at 3 g/min; Temp: 40° C.; Wavelength: 230 nm), R_{T}=5.15 min). ¹H NMR (400 MHz, CDCl₃) δ 7 74 (s, 1H), 7.60 (s, 2H), 7.39 (d, J=7.6 Hz, 2H), 7.17 (d, J=8.4 Hz, 2H), 6.54 (t, J=73.2 Hz, 1H), 6.16-5.95 (m, 1H), 5.84-5.23 (m, 2H), 4.94-4.36 (m, 3H) 3.64-3.60 (m, 1H), 3.38 (s, 2H), 3.12-2.99 (m, 2H) 2.68 (d, J=16.0 Hz, 1H) 1.89 (s, 3H), 1.59-1.57 (m, 3H), 1.29 (d, J=6.0 Hz, 3H). ¹⁹F NMR (376 MHz, CDCl₃) δ −81.32 (s, 2F).

### Compound 13B:

LC-MS (ESI): R_{T}=4.039 min, mass calcd. for C₃₀H₂₉ClF₂N₆O₄S 610.2, m/z found 611.3 [M+H]⁺. Chiral HPLC (Column: Chiralpak 1 N-5.5 μm 4.6*250 mm; Mobile Phase: CO2:MeOH=75:25 at 3 g/min, Temp: 40° C.; Wavelength: 214 nm). R_{T}=6.73 min). ¹H NMR (400 MHz, CDCl₃) 7.75 (s, 1H) 760 (s, 2H), 7.32 (d, J=8.0 Hz, 2H), 7.11 (d, J=8.4 Hz, 2H), 6.52 (t, J=73.6 Hz, 1H), 6.40 (br s, 1H), 5.98 (br s, 1H), 5.53-4.35 (m, 3H), 4.16 (s, 1H), 4.04-3.97 (m, 1H), 3.53-3.46 (m, 1H), 3.41 (dd. J=13.2, 10.0 Hz, 1H) 3.26 (dd. J=13.2, 4.4 Hz, 1H), 3.08 br s, 1H), 2.69 (d, J=16.0 Hz, 1H), 2.00 (s, 3H), 1.59-1.56 (m, 1H), 1.30 (d, J=6.0 Hz, 3H). ¹⁹F NMR (376 MHz, CDCl₃) δ −81.01 (s, 2F).

### Compound 13C:

LC-MS (ESI): R_{T}=4.124 min, mass calcd. for C₃₀H₂₉ClF₂N₆O₄S 610.2, m/z found 611.3 [M+H]⁺. Chiral HPLC (Column: Chiralpak IB N-5.5 μm 4.6*250 mm; Mobile Phase: CO₂:MeOH=75:25 at 3 g/min; Temp: 40° C.; Wavelength: 214 nm), R¹=7.78 min), ¹H NMR (400 MHz, CDCl₃) δ 7.74 (s, 1H), 7.61 (s, 2H), 7.34 (d, J=80 Hz, 2H), 7.12 (d, J=8.4 Hz, 2H), 6.53 (t, J=73.6 Hz, 1H), 6.37-6.30 (m, 1H), 5.98 (br s, 1H), 5.83-5.31 (m, 1H), 4.33-4.31 (m, 2H), 4.22-4.12 (m 1H), 4.03-3.96 (m, 1H), 3.60-3.48 (m, 1H), 3.41 (dd, J=1.18, 10.4 Hz, 1H), 3.27 (dd, J=12.8, 4.4 Hz, 1H), 3.08 (br s, 1H), 2.70 (d, J=16.0 Hz, 1H), 2.00 (s, 3H), 1.55 (d, J=6.8 Hz, 3R), 1.30 (d, J=6.4 Hz, 3H). ¹⁹F NMR 076 MHz, CDCl₃) δ −80.99 (s, 2F).

### Compound 13D:

LC-MS (ESI): R_{T}=3.556 min, mass calcd. for C₃₀H₂₉ClF₂N₆O₄S 610.2, m/z found 611.2 [M+H]⁺. Chiral HPLC (Column: Chiralpak IA 5 μm 4.6*250 mm, Mobile Phase: CO₂:EtOH=60:40 at 3 g/min; Temp: 40° C.; Wavelength: 214 nm), R_{T}=8.23 min). ¹H NMR (400 MHz, CDCl₃) δ 7 75 (s, 1H), 7.61 (s, 2H), 7.36 (d, J=7.2 Hz, 2H), 7.15 (d, J=8.4 Hz, 2H), 6.53 (d, J=73.6 Hz, 1H), 6.03 br s, 1H), 5.79-5.25 (m, 2H), 4.87-4.33 (m, 3H), 3.59 (dd, =13.2, 4.4 Hz, 1H), 3.53-3.30 (m, 2H), 3.06 (dd, J=13.2, 7.2 Hz, 2H), 2.68 (d, J=16.0 Hz, 1H), 1.90 (s, 3H), 1.65-153 (m, 3H), 1.28 (d, J=0.0 z, 3H). ¹⁹F NMR (376 MHz, CDCl₃) δ −81.30 (a, 2F).

### Anti-HBV Activity of Compounds of Formula (I)

### Procedure

The anti HBV activity was measured using the HepG2.117 cell line, a stable, inducibly HBV producing cell line, which replicates HBV in the absence of doxycycline (Tet-off system). The HepG2 cell line is available from ATCCR under number HB-8065 Transfection of the HepG2 cell line can be as described in Sun and Nassal 2006 Journal of Hepatology 45 (2006) 636-645 “Stable HepG2- and Huh7-based human hepatoma cell lines for efficient regulated expression of infectious hepatitis B virus”.

For the antiviral assay, HBV replication was induced, followed by a treatment with serially diluted compound in 96-well plates. After 3 days of treatment, the antiviral activity was determined by quantification of intracellular HBV DINA using real-time PCR and an HBV specific primer set and probe.

Cytotoxicity of the compounds was tested using HepG2 or HepG2 117 cells, incubated for 3 or 4 days in the presence of compounds. The viability of the cells was assessed using the PERKIN ELMER ATPlite Luminescence Assay System.”

### Results

N. D=not determined
CC50 values 3-days incubation unless marked with * (*=4-days incubation)

| Compound Number | EC50 (μM, mean value) | CC50 (μM, mean value) |
|---|---|---|
| 1B | 1.477 | >50.0 |
| 1D | 0.491 | >50.0 |
| 2B | 0.183 | >50.0 |
| 3A | 0.427 | >50.0 |
| 4 | 0.227 | 25.0 |
| 5 | 0.414 | >50.0 |
| 6A | 0.032 | 4.5 |
| 7A | 0.194 | 46.3 |
| 7B | 0.262 | >50.0 |
| 8A | 0.067 | >50.0 |
| 9A | 0.553 | >50.0 |
| 9B | 0.13 | >50.0 |
| 10A | 0.072 | 32.8 |
| 10B | 0.294 | >50.0 |
| 11B | 0.058 | >50.0 |
| 12 | 0.068 | 24.9 |
| 13B | 0.167 | >50.0 |
| 13D | 0.344 | >50.0 |

### Induction or Non-Induction of HBc Speckling

HepG2.117 cells were, cultured in the presence of DMSO or test compound in absence of doxycycline.

After formaldehyde fixation and TritonX-100 permeabilization. Hepatitis B virus core protein (HBc) was immunolabeled with a primary anti-HBc antibody. ALEXA 488-conjugated secondary antibody was used for fluorescent detection of the primary HBV Core signal, CELLMASK Deep Red and HOECHST 33258 were used for the detection of cytoplasm and nucleus respectively, which allowed the segmentation of cellular compartments.

An image analysis software that allows to detect different morphological phenotypes was used to determine the level of HBV core in the cytoplasm or nucleus (high content imaging assay).

1. A compound of Formula (I), 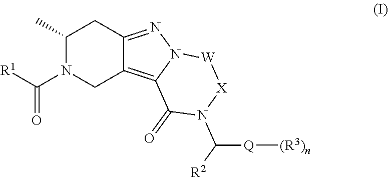
or a stereoisomeric or a tautomeric form thereof, wherein
R¹ is selected from the group consisting of phenyl, pyridyl, pyrimidinyl, pyridazinyl and pyrazinyl, each of which is substituted with 1, 2 or 3 substituents, each of said substituents independently selected from the group consisting of halo, C₁₋₄alkyl, C₃₋₆cycloalkyl, CN, CF₃, CHF₂, OCHF₂ and OCF₃;
R² is hydrogen or a substituent selected from the group consisting of CHF₂, CF₃, C₁₋₄alkyl, C₁₋₄alkylOC₁₋₄alkyl and C₃₋₆cycloalkyl;
Q represents a ring selected from the group consisting of phenyl, a five-membered aromatic heterocyclic ring, and a six-membered aromatic heterocyclic ring;
n represents 1, 2 or 3;
each R³ independently represents a substituent selected from the group consisting of CF₃, CHF₂, CH₂F, C₁₋₆alkyl, OC₁₋₆alkyl, OCF₃, OCHF₂, and C₃₋₆cycloalkyl;
in the event that n represents 2 or 3, two R³ on adjacent ring atoms, together with said ring atoms, optionally form a 5-membered ring or 6-membered ring, said ring optionally comprising 1, 2, or 3 heteroatoms each independently selected from N, O and S, said ring optionally carrying one or more fluoro or oxo substituents;
W is CHR⁴ and X is CHR⁵, wherein R⁴ and R⁵ each independently are selected from the group consisting of hydrogen; CONR⁶R⁷; phenyl; 5-membered heterocyclyl; 6-membered heterocyclyl; 5-membered heteroaryl; 6-membered heteroaryl; C₃₋₆cycloalkyl; C₁₋₄alkyl; and C₁₋₄alkyl substituted with OH, OC₁₋₄alkyl, halo, COOH, CONR⁸R⁹ NHCOC₁₋₄alkyl, NHCOC₃₋₆cycloalkyl, 5-membered heteroaryl, 6-membered heteroaryl, SO₂NHC₁₋₄alkyl, SO₂NHC₃₋₆cycloalkyl, NHSO₂C₁₋₄alkyl or NHSO₂C₃₋₆cycloalkyl; wherein R⁴ and R⁵ are not both hydrogen;
R⁶ and R⁷ each independently are selected from hydrogen, C₃₋₆cycloalkyl, C₁₋₄alkyl, C₁₋₄alkyl substituted with halo, OH or OC₁₋₄alkyl, or R⁶ and R⁷ are taken together along with a nitrogen to which R⁶ and R⁷ are attached to form an optionally substituted 4 to 8 membered monocyclic heterocyclyl;
R⁸ and R⁹ each independently are selected from hydrogen, C₃₋₆cycloalkyl, C₁₋₄alkyl, C₁₋₄alkyl substituted with halo, OH, or OC₁₋₄alkyl, or R⁸ and R⁹ are taken together along with a nitrogen to which R⁸ and R⁹ are attached to form an optionally substituted 4 to 8 membered monocyclic heterocyclyl;
or W and X together are CR¹⁰═N or CR¹¹═CR¹²;
R¹⁰, R¹¹ and R¹² each independently are selected from the group consisting of hydrogen, halo, CONR¹³R¹⁴, phenyl, 5-membered heterocyclyl, 6-membered heterocyclyl, 5-membered heteroaryl, 6-membered heteroaryl, C₃₋₆cycloalkyl, C₁₋₄alkyl, and C₁₋₄alkyl substituted with OH, OC₁₋₄alkyl, halo, COOH, CONR¹⁵R¹⁶, NHCOC₁₋₄alkyl, NHCOC₃₋₆cycloalkyl, SO₂NHC₁₋₄alkyl, SO₂NHC₃₋₆cycloalkyl, NHSO₂C₁₋₄alkyl or NHSO₂C₃₋₆cycloalkyl;
R¹³ and R¹⁴ each independently are selected from hydrogen, C₁₋₄alkyl, C₃₋₆cycloalkyl, or R¹³ and R¹⁴ are taken together along with a nitrogen to which R¹³ and R¹⁴ are attached to form an optionally substituted 4- to 8-membered monocyclic heterocyclyl;
R¹⁵ and R¹⁶ each independently are selected from hydrogen, C₁₋₄alkyl, C₃₋₆cycloalkyl, or R¹⁵ and R¹⁶ are taken together along with a nitrogen to which R¹⁵ and R¹⁶ are attached to form an optionally substituted 4 to 8 membered monocyclic heterocyclyl;
or a pharmaceutically acceptable salt or a solvate thereof.

2. The compound of claim 1, wherein the structure of Formula (I) satisfies Formula (IA) 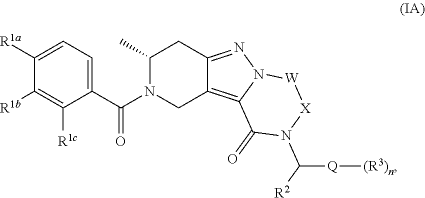
wherein R^{1a}, R^{1b}, and R^{1c}, each independently are selected from the group consisting of hydrogen, halo, C₁₋₄alkyl, C₃₋₆cycloalkyl, CN, CF₃, CHF₂, OCHF₂ and OCF₃, with at least one of R^{1a}, R^{1b}, and R^{1c} not being hydrogen.

3. The compound of claim 2, wherein the structure of Formula (IA) satisfies Formula (IB)

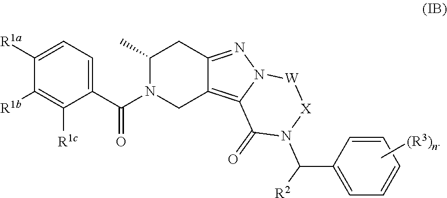

4. The compound of claim 3, wherein the structure of Formula (IB) satisfies Formula (IE)

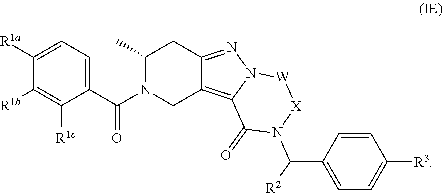

5. The compound of claim 1, wherein the structure of Formula (I) satisfies Formula (IC)

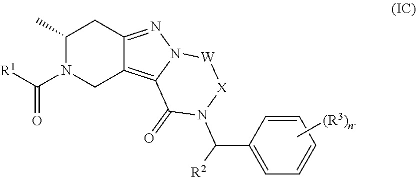

6. The compound of claim 5, wherein the structure of Formula (IC) satisfies Formula (ID)

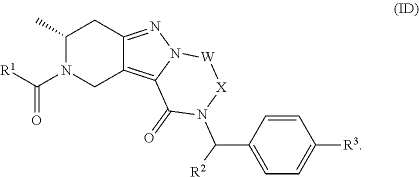

7. The compound of claim 1, wherein one of R⁴ and R⁵ is H, and the other one is selected from the group consisting of CONHC₁₋₄alkyl, C₁₋₄alkyl, C₁₋₄alkyl substituted with OH, and C₁₋₄alkyl substituted with NHCOC₁₋₄alkyl.

8. The compound of claim 1, wherein W and X together are CH═N or CH═CH.

9. The compound of claim 2, wherein R^{1a} is halo, R^{1b} is selected from the group consisting of halo and cyano, and wherein R^{1c} is hydrogen.

10. The compound of claim 9, wherein R^{1b} is halo, and the halo is choro.

11. The compound of claim 1, wherein n is 1.

12. The compound of claim 1, wherein one R³ is OCHF₂.

13. A compound selected from the group consisting of the following compounds 1-13 or a stereoisomeric or a tautomeric form thereof: 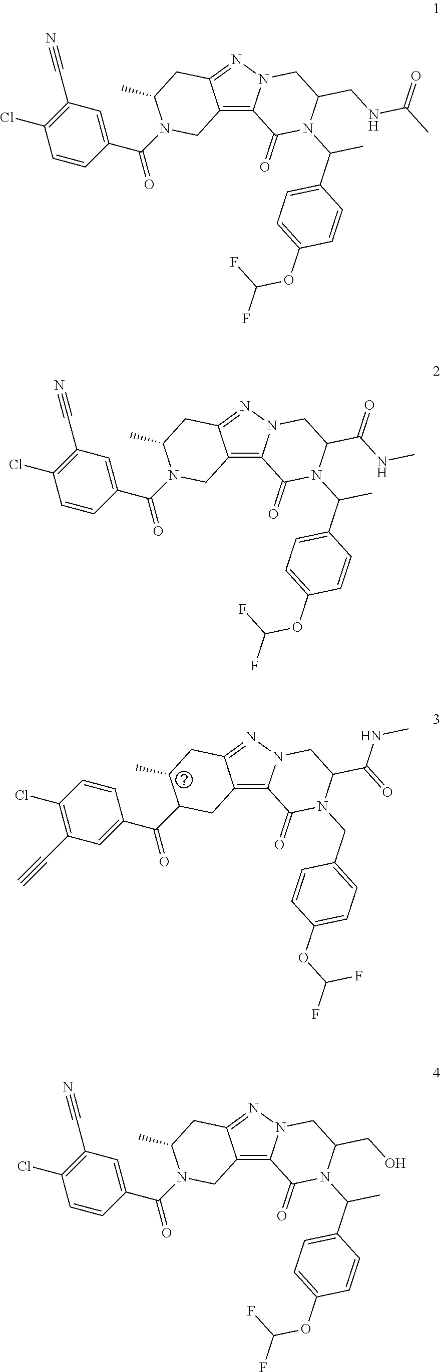 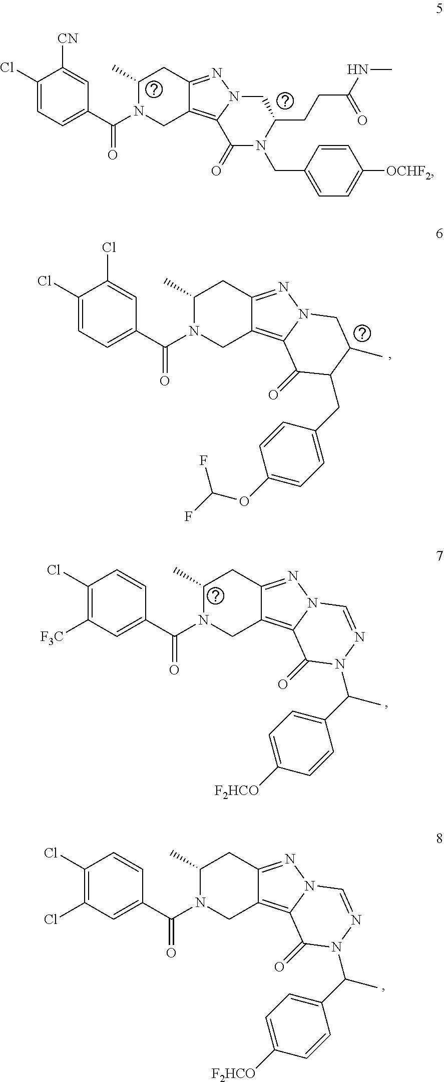 [SMILES: CNC(=O)CC[C@H]1Cn2nc3c(c2C(=O)N1Cc1ccc(OC(F)F)cc1)CN(C(=O)c1ccc(Cl)c(C#N)c1)[C@H](C)C3 ; CC1Cn2nc3c(c2C(=O)C1Cc1ccc(OC(F)F)cc1)CN(C(=O)c1ccc(Cl)c(Cl)c1)[C@H](C)C3 ; CC(c1ccc(OC(F)F)cc1)n1ncn2nc3c(c2c1=O)CN(C(=O)c1ccc(Cl)c(C(F)(F)F)c1)[C@H](C)C3 ; CC(c1ccc(OC(F)F)cc1)n1ncn2nc3c(c2c1=O)CN(C(=O)c1ccc(Cl)c(Cl)c1)[C@H](C)C3] 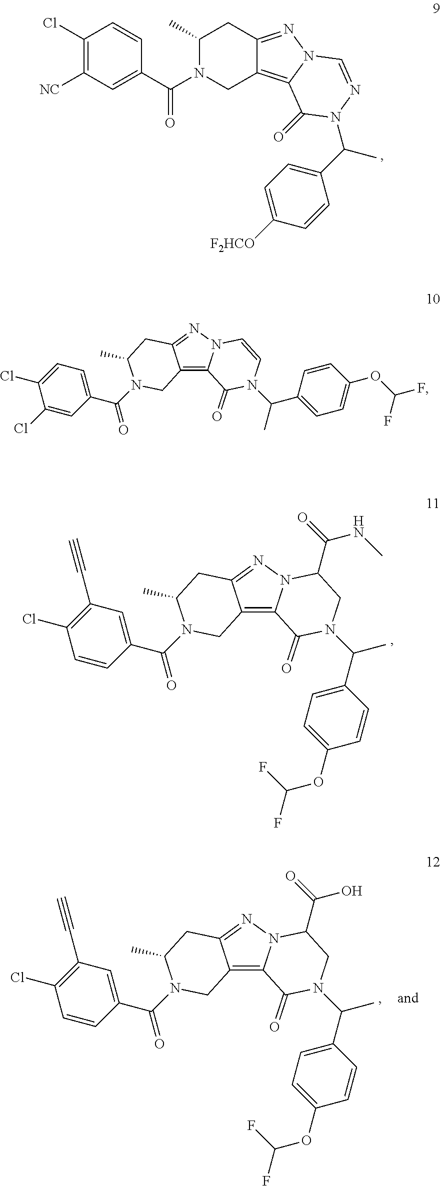 [SMILES: CC(c1ccc(OC(F)F)cc1)n1ncn2nc3c(c2c1=O)CN(C(=O)c1ccc(Cl)c(C#N)c1)[C@H](C)C3 ; CC(c1ccc(OC(F)F)cc1)n1ccn2nc3c(c2c1=O)CN(C(=O)c1ccc(Cl)c(Cl)c1)[C@H](C)C3 ; C#Cc1cc(C(=O)N2Cc3c(nn4c3C(=O)N(C(C)c3ccc(OC(F)F)cc3)CC4C(=O)NC)C[C@H]2C)ccc1Cl ; C#Cc1cc(C(=O)N2Cc3c(nn4c3C(=O)N(C(C)c3ccc(OC(F)F)cc3)CC4C(=O)O)C[C@H]2C)ccc1Cl] 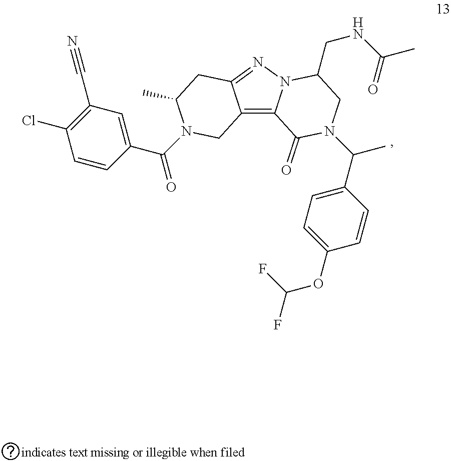 [SMILES: CC(=O)NCC1CN(C(C)c2ccc(OC(F)F)cc2)C(=O)c2c3c(nn21)C[C@@H](C)N(C(=O)c1ccc(Cl)c(C#N)c1)C3]
or a pharmaceutically acceptable salt, N-oxide, or a solvate thereof.

14. A pharmaceutical composition, which comprises the compound of claim 1, and which further comprises at least one pharmaceutically acceptable excipient.

15. (canceled)

16. (canceled)

17. (canceled)

18. A method of treating an HBV infection or an HBV-induced disease in an individual in need thereof, comprising administering to the individual a therapeutically effective amount of the compound of claim 1.

19. A product comprising a first compound and a second compound as a combined preparation for simultaneous, separate or sequential use in the prevention or treatment of an HBV infection or of an HBV-induced disease in a subject in need thereof, wherein said first compound is different from said second compound, wherein said first compound is the compound of claim 1, and wherein said second compound is another HBV inhibitor.

20. The product of claim 19, wherein said second compound is another HBV inhibitor which is selected from the group consisting of: therapeutic agents selected from HBV combination drugs, HBV vaccines, HBV DNA polymerase inhibitors, immunomodulatory agents, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (CTLA-4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and other HBV drugs.

21. (canceled)

22. The method of claim 18, wherein the HBV infection or an HBV-induced disease is chronic hepatitis B.

23. The method of claim 18, wherein the compound is administered to the subject in combination with another HBV inhibitor.
